SEQUENCE LISTING (132 sequences)

SEQ ID NO: 1 - protein, 19 aa, Artificial Sequence
  Synthetic Construct
TFVYGGCRAKRNNFKSAED

SEQ ID NO: 2 - protein, 19 aa, Artificial Sequence
  Synthetic Construct
TFQYGGCMGNGNNFVTEKE

SEQ ID NO: 3 - protein, 19 aa, Artificial Sequence
  Synthetic Construct
PFFYGGCGGNRNNFDTEEY

SEQ ID NO: 4 - protein, 19 aa, Artificial Sequence
  Synthetic Construct
SFYYGGCLGNKNNYLREEE

SEQ ID NO: 5 - protein, 19 aa, Artificial Sequence
  Synthetic Construct
TFFYGGCRAKRNNFKRAKY

SEQ ID NO: 6 - protein, 19 aa, Artificial Sequence
  Synthetic Construct
TFFYGGCRGKRNNFKRAKY

SEQ ID NO: 7 - protein, 19 aa, Artificial Sequence
  Synthetic Construct
TFFYGGCRAKKNNYKRAKY

SEQ ID NO: 8 - protein, 19 aa, Artificial Sequence
  Synthetic Construct
TFFYGGCRGKKNNFKRAKY

SEQ ID NO: 9 - protein, 19 aa, Artificial Sequence
  Synthetic Construct
TFQYGGCRAKRNNFKRAKY

SEQ ID NO: 10 - protein, 19 aa, Artificial Sequence
  Synthetic Construct
TFQYGGCRGKKNNFKRAKY

SEQ ID NO: 11 - protein, 19 aa, Artificial Sequence
  Synthetic Construct
TFFYGGCLGKRNNFKRAKY

SEQ ID NO: 12 - protein, 19 aa, Artificial Sequence
  Synthetic Construct
TFFYGGSLGKRNNFKRAKY

SEQ ID NO: 13 - protein, 19 aa, Artificial Sequence
  Synthetic Construct
PFFYGGCGGKKNNFKRAKY

SEQ ID NO: 14 - protein, 19 aa, Artificial Sequence
  Synthetic Construct
TFFYGGCRGKGNNYKRAKY

SEQ ID NO: 15 - protein, 19 aa, Artificial Sequence
  Synthetic Construct
PFFYGGCRGKRNNFLRAKY

SEQ ID NO: 16 - protein, 19 aa, Artificial Sequence
  Synthetic Construct
TFFYGGCRGKRNNFKREKY

SEQ ID NO: 17 - protein, 19 aa, Artificial Sequence
  Synthetic Construct
PFFYGGCRAKKNNFKRAKE

SEQ ID NO: 18 - protein, 19 aa, Artificial Sequence
  Synthetic Construct
TFFYGGCRGKRNNFKRAKD

SEQ ID NO: 19 - protein, 19 aa, Artificial Sequence
  Synthetic Construct
TFFYGGCRAKRNNFDRAKY

SEQ ID NO: 20 - protein, 19 aa, Artificial Sequence
  Synthetic Construct
TFFYGGCRGKKNNFKRAEY

SEQ ID NO: 21 - protein, 19 aa, Artificial Sequence
  Synthetic Construct
PFFYGGCGANRNNFKRAKY

SEQ ID NO: 22 - protein, 19 aa, Artificial Sequence
  Syntheic Construct
TFFYGGCGGKKNNFKTAKY

SEQ ID NO: 23 - protein, 19 aa, Artificial Sequence
  Synthetic Construct
TFFYGGCRGNRNNFLRAKY

SEQ ID NO: 24 - protein, 19 aa, Artificial Sequence
  Synthetic Construct
TFFYGGCRGNRNNFKTAKY

SEQ ID NO: 25 - protein, 19 aa, Artificial Sequence
  Synthetic Construct
TFFYGGSRGNRNNFKTAKY

SEQ ID NO: 26 - protein, 19 aa, Artificial Sequence
  Synthetic Construct
TFFYGGCLGNGNNFKRAKY

SEQ ID NO: 27 - protein, 19 aa, Artificial Sequence
  Synthetic Construct
TFFYGGCLGNRNNFLRAKY

SEQ ID NO: 28 - protein, 19 aa, Artificial Sequence
  Synthetic Construct
TFFYGGCLGNRNNFKTAKY

SEQ ID NO: 29 - protein, 19 aa, Artificial Sequence
  Synthetic Construct
TFFYGGCRGNGNNFKSAKY

SEQ ID NO: 30 - protein, 19 aa, Artificial Sequence
  Synthetic Construct
TFFYGGCRGKKNNFDREKY

SEQ ID NO: 31 - protein, 19 aa, Artificial Sequence
  Synthetic Construct
TFFYGGCRGKRNNFLREKE

SEQ ID NO: 32 - protein, 19 aa, Artificial Sequence
  Synthetic Construct
TFFYGGCRGKGNNFDRAKY

SEQ ID NO: 33 - protein, 19 aa, Artificial Sequence
  Synthetic Construct
TFFYGGSRGKGNNFDRAKY

SEQ ID NO: 34 - protein, 19 aa, Artificial Sequence
  Synthetic Construct
TFFYGGCRGNGNNFVTAKY

SEQ ID NO: 35 - protein, 19 aa, Artificial Sequence
  Synthetic Construct
PFFYGGCGGKGNNYVTAKY

SEQ ID NO: 36 - protein, 19 aa, Artificial Sequence
  Synthetic Construct
TFFYGGCLGKGNNFLTAKY

SEQ ID NO: 37 - protein, 19 aa, Artificial Sequence
  Synthetic Construct
SFFYGGCLGNKNNFLTAKY

SEQ ID NO: 38 - protein, 19 aa, Artificial Sequence
  Synthetic Construct
TFFYGGCGGNKNNFVREKY

SEQ ID NO: 39 - protein, 19 aa, Artificial Sequence
  Synthetic Construct
TFFYGGCMGNKNNFVREKY

SEQ ID NO: 40 - protein, 19 aa, Artificial Sequence
  Synthetic Construct
TFFYGGSMGNKNNFVREKY

SEQ ID NO: 41 - protein, 19 aa, Artificial Sequence
  Synthetic Construct
PFFYGGCLGNRNNYVREKY

SEQ ID NO: 42 - protein, 19 aa, Artificial Sequence
  Synthetic Construct
TFFYGGCLGNRNNFVREKY

SEQ ID NO: 43 - protein, 19 aa, Artificial Sequence
  Synthetic Construct
TFFYGGCLGNKNNYVREKY

SEQ ID NO: 44 - protein, 19 aa, Artificial Sequence
  Synthetic Construct
TFFYGGCGGNGNNFLTAKY

SEQ ID NO: 45 - protein, 19 aa, Artificial Sequence
  Synthetic Construct
TFFYGGCRGNRNNFLTAEY

SEQ ID NO: 46 - protein, 19 aa, Artificial Sequence
  Synthetic Construct
TFFYGGCRGNGNNFKSAEY

SEQ ID NO: 47 - protein, 19 aa, Artificial Sequence
  Synthetic Construct
PFFYGGCLGNKNNFKTAEY

SEQ ID NO: 48 - protein, 19 aa, Artificial Sequence
  Synthetic Construct
TFFYGGCRGNRNNFKTEEY

SEQ ID NO: 49 - protein, 19 aa, Artificial Sequence
  Synthetic Construct
TFFYGGCRGKRNNFKTEED

SEQ ID NO: 50 - protein, 19 aa, Artificial Sequence
  Synthetic Construct
PFFYGGCGGNGNNFVREKY

SEQ ID NO: 51 - protein, 19 aa, Artificial Sequence
  Synthetic Construct
SFFYGGCMGNGNNFVREKY

SEQ ID NO: 52 - protein, 19 aa, Artificial Sequence
  Synthetic Construct
PFFYGGCGGNGNNFLREKY

SEQ ID NO: 53 - protein, 19 aa, Artificial Sequence
  Synthetic Construct
TFFYGGCLGNGNNFVREKY

SEQ ID NO: 54 - protein, 19 aa, Artificial Sequence
  Synthetic Construct
SFFYGGCLGNGNNYLREKY

SEQ ID NO: 55 - protein, 19 aa, Artificial Sequence
  Synthetic Construct
TFFYGGSLGNGNNFVREKY

SEQ ID NO: 56 - protein, 19 aa, Artificial Sequence
  Synthetic Construct
TFFYGGCRGNGNNFVTAEY

SEQ ID NO: 57 - protein, 19 aa, Artificial Sequence
  Synthetic Construct
TFFYGGCLGKGNNFVSAEY

SEQ ID NO: 58 - protein, 19 aa, Artificial Sequence
  Synthetic Construct
TFFYGGCLGNRNNFDRAEY

SEQ ID NO: 59 - protein, 19 aa, Artificial Sequence
  Synthetic Construct
TFFYGGCLGNRNNFLREEY

SEQ ID NO: 60 - protein, 19 aa, Artificial Sequence
  Synthetic Construct
TFFYGGCLGNKNNYLREEY

SEQ ID NO: 61 - protein, 19 aa, Artificial Sequence
  Synthetic Construct
PFFYGGCGGNRNNYLREEY

SEQ ID NO: 62 - protein, 19 aa, Artificial Sequence
  Synthetic Construct
PFFYGGSGGNRNNYLREEY

SEQ ID NO: 63 - protein, 19 aa, Artificial Sequence
  Synthetic Construct
MRPDFCLEPPYTGPCVARI

SEQ ID NO: 64 - protein, 21 aa, Artificial Sequence
  Synthetic Construct
ARIIRYFYNAKAGLCQTFVYG

SEQ ID NO: 65 - protein, 22 aa, Artificial Sequence
  Synthetic Construct
YGGCRAKRNNYKSAEDCMRTCG

SEQ ID NO: 66 - protein, 22 aa, Artificial Sequence
  Synthetic Construct
PDFCLEPPYTGPCVARIIRYFY

SEQ ID NO: 67 - protein, 19 aa, Artificial Sequence
  Synthetic Construct
TFFYGGCRGKRNNFKTEEY

SEQ ID NO: 68 - protein, 19 aa, Artificial Sequence
  Synthetic Construct
KFFYGGCRGKRNNFKTEEY

SEQ ID NO: 69 - protein, 19 aa, Artificial Sequence
  Synthetic Construct
TFYYGGCRGKRNNYKTEEY

SEQ ID NO: 70 - protein, 19 aa, Artificial Sequence
  Synthetic Construct
TFFYGGSRGKRNNFKTEEY

SEQ ID NO: 71 - protein, 20 aa, Artificial Sequence
  Synthetic Construct
CTFFYGCCRGKRNNFKTEEY

SEQ ID NO: 72 - protein, 20 aa, Artificial Sequence
  Synthetic Construct
TFFYGGCRGKRNNFKTEEYC

SEQ ID NO: 73 - protein, 20 aa, Artificial Sequence
  Synthetic Construct
CTFFYGSCRGKRNNFKTEEY

SEQ ID NO: 74 - protein, 20 aa, Artificial Sequence
  Synthetic Construct
TFFYGGSRGKRNNFKTEEYC

SEQ ID NO: 75 - protein, 19 aa, Artificial Sequence
  Synthetic Construct
PFFYGGCRGKRNNFKTEEY

SEQ ID NO: 76 - protein, 19 aa, Artificial Sequence
  Synthetic Construct
TFFYGGCRGKRNNFKTKEY

SEQ ID NO: 77 - protein, 19 aa, Artificial Sequence
  Synthetic Construct
TFFYGGKRGKRNNFKTEEY

SEQ ID NO: 78 - protein, 19 aa, Artificial Sequence
  Synthetic Construct
TFFYGGCRGKRNNFKTKRY

SEQ ID NO: 79 - protein, 19 aa, Artificial Sequence
  Synthetic Construct
TFFYGGKRGKRNNFKTAEY

SEQ ID NO: 80 - protein, 19 aa, Artificial Sequence
  Synthetic Construct
TFFYGGKRGKRNNFKTAGY

SEQ ID NO: 81 - protein, 19 aa, Artificial Sequence
  Synthetic Construct
TFFYGGKRGKRNNFKREKY

SEQ ID NO: 82 - protein, 19 aa, Artificial Sequence
  Synthetic Construct
TFFYGGKRGKRNNFKRAKY

SEQ ID NO: 83 - protein, 19 aa, Artificial Sequence
  Synthetic Construct
TFFYGGCLGNRNNFKTEEY

SEQ ID NO: 84 - protein, 19 aa, Artificial Sequence
  Synthetic Construct
TFFYGCGRGKRNNFKTEEY

SEQ ID NO: 85 - protein, 19 aa, Artificial Sequence
  Synthetic Construct
TFFYGGRCGKRNNFKTEEY

SEQ ID NO: 86 - protein, 19 aa, Artificial Sequence
  Synthetic Construct
TFFYGGCLGNGNNFDTEEE

SEQ ID NO: 87 - protein, 19 aa, Artificial Sequence
  Synthetic Construct
TFQYGGCRGKRNNFKTEEY

SEQ ID NO: 88 - protein, 19 aa, Artificial Sequence
  Synthetic Construct
YNKEFGTFNTKGCERGYRF

SEQ ID NO: 89 - protein, 19 aa, Artificial Sequence
  Synthetic Construct
RFKYGGCLGNMNNFETLEE

SEQ ID NO: 90 - protein, 19 aa, Artificial Sequence
  Synthetic Construct
RFKYGGCLGNKNNFLRLKY

SEQ ID NO: 91 - protein, 19 aa, Artificial Sequence
  Synthetic Construct
RFKYGGCLGNKNNYLRLKY

SEQ ID NO: 92 - protein, 22 aa, Artificial Sequence
  Synthetic Construct
KTKRKRKKQRVKIAYEEIFKNY

SEQ ID NO: 93 - protein, 15 aa, Artificial Sequence
  Synthetic Construct
KTKRKRKKQRVKIAY

SEQ ID NO: 94 - protein, 17 aa, Artificial Sequence
  Synthetic Construct
RGGRLSYSRRFSTSTGR

SEQ ID NO: 95 - protein, 10 aa, Artificial Sequence
  Synthetic Construct
RRLSYSRRRF

SEQ ID NO: 96 - protein, 16 aa, Artificial Sequence
  Synthetic Construct
RQIKIWFQNRRMKWKK

SEQ ID NO: 97 - protein, 19 aa, Artificial Sequence
  Synthetic Construct
TFFYGGSRGKRNNFKTEEY

SEQ ID NO: 98 - protein, 59 aa, Artificial Sequence
  Synthetic Construct
MRPDFCLEPPYTGPCVARIIRYFYNAKAGLCQTFVYGGCRAKRNNFKSAEDCMRTCGGA

SEQ ID NO: 99 - protein, 19 aa, Artificial Sequence
  Synthetic Construct
TFFYGGCRGKRNNFKTKEY

SEQ ID NO: 100 - protein, 19 aa, Artificial Sequence
  Synthetic Construct
RFKYGGCLGNKNNYLRLKY

SEQ ID NO: 101 - protein, 19 aa, Artificial Sequence
  Synthetic Construct
TFFYGGCRAKRNNFKRAKY

SEQ ID NO: 102 - protein, 35 aa, Artificial Sequence
  Synthetic Construct
NAKAGLCQTFVYGGCLAKRNNFESAEDCMRTCGGA

SEQ ID NO: 103 - protein, 24 aa, Artificial Sequence
  Synthetic Construct
YGGCRAKRNNFKSAEDCMRTCGGA

SEQ ID NO: 104 - protein, 22 aa, Artificial Sequence
  Synthetic Construct
GLCQTFVYGGCRAKRNNFKSAE

SEQ ID NO: 105 - protein, 20 aa, Artificial Sequence
  Synthetic Construct
LCQTFVYGGCEAKRNNFKSA

SEQ ID NO: 106 - skipped

SEQ ID NO: 107 - protein, 19 aa, Artificial Sequence
  Synthetic Construct
TFFYGGSRGKRNNFKTEEY

SEQ ID NO: 108 - protein, 19 aa, Artificial Sequence
  Synthetic Construct
RFFYGGSRGKRNNFKTEEY

SEQ ID NO: 109 - protein, 19 aa, Artificial Sequence
  Synthetic Construct
RFFYGGSRGKRNNFKTEEY

SEQ ID NO: 110 - protein, 19 aa, Artificial Sequence
  Synthetic Construct
RFFYGGSRGKRNNFRTEEY

SEQ ID NO: 111 - protein, 19 aa, Artificial Sequence
  Synthetic Construct
TFFYGGSRGKRNNFRTEEY

SEQ ID NO: 112 - protein, 19 aa, Artificial Sequence
  Synthetic Construct
TFFYGGSRGRRNNFRTEEY

SEQ ID NO: 113 - protein, 20 aa, Artificial Sequence
  Synthetic Construct
CTFFYGGSRGKRNNFKTEEY

SEQ ID NO: 114 - protein, 20 aa, Artificial Sequence
  Synthetic Construct
TFFYGGSRGKRNNFKTEEYC

SEQ ID NO: 115 - protein, 20 aa, Artificial Sequence
  Synthetic Construct
CTFFYGGSRGRRNNFRTEEY

SEQ ID NO: 116 - protein, 20 aa, Artificial Sequence
  Synthetic Construct
TFFYGGSRGRRNNFRTEEYC

SEQ ID NO: 117 - protein, 19 aa, Artificial Sequence
  Synthetic Construct
YEETKFNNRKGRSGGYFFT

SEQ ID NO: 118 - protein, 6 aa, Artificial Sequence
  Description of Artificial Sequence: Synthetic peptide
  MOD_RES (1)..(6): optionally comprises one or more D-isomers of a recited amino acid
  MOD_RES (3)..(3): Asn or Gln
  MOD_RES (4)..(4): Asn or Gln
  MOD_RES (5)..(5): Phe, Tyr, or Trp
KRXXXK

SEQ ID NO: 119 - protein, 8 aa, Artificial Sequence
  Description of Artificial Sequence: Synthetic peptide
  MOD_RES (1)..(1): absent, Cys, Gly, Cys-Gly, Arg-Gly, Cys-Arg- Gly, Ser-Arg-Gly, Cys-Ser-Arg-Gly, Gly-Ser-Arg-Gly, Cys-Gly-Ser- Arg-Gly, Gly-Gly-Ser-Arg-Gly, Cys-Gly-Gly-Ser-Arg-Gly, Tyr-Gly-Gly-Ser-Arg-Gly, or Cys-Tyr-Gly-Gly-Ser-Arg-Gly
  MOD_RES (1)..(1): Phe-Tyr-Gly-Gly-Ser-Arg-Gly, Cys-Phe-Tyr-Gly- Gly-Ser-Arg-Gly, Phe-Phe-Tyr-Gly-Gly-Ser-Arg-Gly, or Cys-Phe-Phe-Tyr-Gly-Gly-Ser-Arg-Gly
  MOD_RES (1)..(1): Thr-Phe-Phe-Tyr-Gly-Gly-Ser-Arg-Gly or Cys-Thr-Phe-Phe-Tyr-Gly-Gly-Ser-Arg-Gly
  MOD_RES (1)..(8): optionally comprises one or more D-isomers of a recited amino acid
  MOD_RES (4)..(4): Asn or Gln
  MOD_RES (5)..(5): Asn or Gln
  MOD_RES (6)..(6): Phe, Tyr, or Trp
  MOD_RES (8)..(8): absent, Cys, Tyr, Tyr-Cys, Cys-Tyr, Thr-Glu- Glu-Tyr, or Thr-Glu-Glu-Tyr-Cys
XKRXXXKX

SEQ ID NO: 120 - protein, 6 aa, Artificial Sequence
  Description of Artificial Sequence: Synthetic peptide
  MOD_RES (1)..(1): Lys or D-Lys
  MOD_RES (1)..(6): at least one of X1, X2, X5, or X6 is a D-amino acid
  MOD_RES (2)..(2): Arg or D-Arg
  MOD_RES (5)..(5): Phe or D-Phe
  MOD_RES (6)..(6): Lys or D-Lys
XXNNXX

SEQ ID NO: 121 - protein, 7 aa, Artificial Sequence
  Description of Artificial Sequence: Synthetic peptide
  MOD_RES (1)..(1): Lys or D-Lys
  MOD_RES (1)..(7): at least one of X1, X2, X5, X6, or X7 is a D-amino acid
  MOD_RES (2)..(2): Arg or D-Arg
  MOD_RES (5)..(5): Phe or D-Phe
  MOD_RES (6)..(6): Lys or D-Lys
  MOD_RES (7)..(7): Tyr or D-Tyr
XXNNXXX

SEQ ID NO: 122 - protein, 9 aa, Artificial Sequence
  Synthetic Construct
  MOD_RES (1)..(1): absent, Cys, Gly, Cys-Gly, Arg-Gly, Cys-Arg- Gly, Ser-Arg-Gly, Cys-Ser-Arg-Gly, Gly-Ser-Arg-Gly, Cys-Gly-Ser- Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly,
  MOD_RES (1)..(1): Cys-Phe-Tyr-Gly-Gly-Ser-Arg-Gly, Phe-Phe-Tyr- Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly
  MOD_RES (2)..(2): Lys or D-Lys
  MOD_RES (3)..(3): Arg or D-Arg
  MOD_RES (6)..(6): Phe or D-Phe
  MOD_RES (7)..(7): Lys or D-Lys
  MOD_RES (8)..(8): Tyr or D-Tyr
  MOD_RES (9)..(9): absent, Cys, Tyr, Tyr-Cys, Cys-Tyr, Thr-Glu- Glu-Tyr, or Thr-Glu-Glu-Tyr-Cys
XXXNNXXXX

SEQ ID NO: 123 - protein, 6 aa, Artificial Sequence
  Synthetic Construct
KRNNFK

SEQ ID NO: 124 - protein, 7 aa, Artificial Sequence
  Synthetic Construct
KRNNFKY

SEQ ID NO: 125 - protein, 8 aa, Artificial Sequence
  Synthetic Construct
KRNNFKYC

SEQ ID NO: 126 - protein, 5 aa, Artificial Sequence
  Synthetic Construct
GGGGS

SEQ ID NO: 127 - protein, 5 aa, Artificial Sequence
  Synthetic Construct
  misc_feature (1)..(4): Xaa can be Ser or Thr, up to two of the X are Thr, and the remaining X are Ser
XXXXG

SEQ ID NO: 128 - protein, 20 aa, Artificial Sequence
  Synthetic Construct
  MOD_RES (1)..(1): Azidobutyryl
XTFFYGGSRGKRNNFKTEEY

SEQ ID NO: 129 - protein, 20 aa, Artificial Sequence
  Synthetic Construct
  MOD_RES (20)..(20): azido-norleucine
TFFYGGSRGKRNNFKTEEYX

SEQ ID NO: 130 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 131 - protein, 343 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGFLMRTNT

SEQ ID NO: 132 - protein, 460 aa, Homo sapiens
MPLRRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

(19) United States
(12) Patent Application Publication
(10) Pub. No.: US 2015/0290341 A1
(43) Pub. Date: Oct. 15, 2015

(54) TARGETED IDURONATE-2-SULFATASE COMPOUNDS

(71) Applicant: ANGIOCHEM INC., Montréal (CA)

(72) Inventors: Dominique Boivin, Sainte-Marthe- Sur-Le-Lac (CA); Jean-Paul Castaigne, Mont-Royal (CA); Michel Demeule, Beaconsfield (CA); Sasmita Tripathy, Pierre Fonds (CA); Jean-Christophe Currie, Repentigny (CA); Simon Lord-Dufour, Montreal (CA)

(73) Assignee: Anglachem Inc., Montreal (CA)

(21) Appl. No.: 14/648,654

(22) PCT Filed: Dec. 2, 2013
(86) PCT No.: PCT/CA2013/050924

§ 371 Date: May 29, 2015

Related U.S. Application Data
(60) Provisional application No. 61/831,919, filed on Jun. 6, 2013.
(60) Provisional application No. 61/732,145, filed on Nov. 30, 2012.

(51) Int. Cl. A61K 47/48; C12N 9/16; A61K 38/46

(52) U.S. Cl. CPC A61K 47/48346; A61K 38/465; C12N 9/16; C12Y 301/06013

(57) ABSTRACT

The present invention is related to a compound that includes a lysosomal enzyme and a targeting moiety, for example, a compound that includes iduronate-2-sulfatase conjugated to Angiopep-2 through a linker formed by specific click chemistry reactions. In certain embodiments, these compounds, owing to the presence of the targeting moiety, can cross the blood-brain barrier or accumulate in the lysosome more effectively than the enzyme alone. The invention also features pharmaceutical compositions containing such compounds and methods for treating lysosomal storage disorders (e.g., mucopolysaccharidosis Type II) using such compounds.

### CROSS-REFERENCE TO RELATED APPLICATIONS

This application claims benefit of U.S. Provisional Application Nos. 61/732,145, filed Nov. 30, 2012, and 61/831,919 filed Jun. 6, 2013, which are hereby incorporated by reference in their entirety.

### BACKGROUND OF THE INVENTION

The invention relates to compounds including a lysosomal enzyme and a targeting moiety and the use of such compounds in the treatment of disorders that result from a deficiency of such enzymes. The invention additionally relates to modified lysosomal enzyme intermediates in the production of these compounds.

Lysosomal storage disorders are group of about 50 rare genetic disorders in which a subject has a defect in a lysosomal enzyme that is required for proper metabolism. These diseases typically result from autosomal or X-linked recessive genes. As a group, the incidence of these disorders is about 1:5000 to 1:10,000.

Hunter syndrome, or mucopolysaccharidosis Type II (MPS-II), results from a deficiency of iduronate-2-sulfatase (IDS; also known as idursulfase), an enzyme that is required for lysosomal degradation of heparin sulfate and dermatan sulfate. Because the disorder is X-linked recessive, it primarily affects males. Those with the disorder are unable to break down and recycle these mucopolysaccharides, which are also known as glycosaminoglycans or GAGs. This deficiency results in the buildup of GAG throughout the body, which has serious effects on the nervous system, joints, and various organ systems, including the heart, liver, and skin. There are also a number of physical symptoms, including coarse facial features, enlarged head and abdomen, and skin lesions. In the most severe cases, the disease can be fatal in teen years and is accompanied by severe mental retardation.

There is no cure for MPS-II. In addition to palliative measures, therapeutic approaches have included bone marrow grafts and enzyme replacement therapy. Bone marrow grafts have been observed to stabilize the peripheral symptoms of MPS-II, including cardiovascular abnormalities, hepatosplenomegaly (enlarged liver and spleen), and joint stiffness. This approach, however, did not stabilize or resolve the neuropsychological symptoms associated with this disease (Guffon et al., J. Pediatr. 154:733-7, 2009).

Enzyme replacement therapy by intravenous administration of IDS has also been shown to have benefits, including improvement in skin lesions (Marin et al., (published online ahead of print) Pediatr. Dermatol. Oct. 13, 2011), visceral organ size, gastrointestinal functioning, and reduced need for antibiotics to treat upper airway infections (Hoffman et al., Pediatr. Neurol. 45:181-4, 2011). Like bone marrow grafts, this approach does not improve the central nervous system deficits associated with MPS-II because the enzyme is not expected to cross the blood-brain barrier (BBB; Wraith et al., Eur. I Pediatr. 1676:267-7, 2008).

Methods for increasing delivery of IDS to the brain have been and are being investigated, including intrathecal delivery (Felice et al., Toxicol. Pathol. 39:879-92, 2011). Intrathecal delivery, however, is a highly invasive technique.

Less invasive and more effective methods of treating MPS-II that address the neurological disease symptoms, in addition to the other symptoms, would therefore be highly desirable.

### SUMMARY OF THE INVENTION

The present invention is directed to compounds that include a targeting moiety and a lysosomal enzyme. These compounds are exemplified by IDS-Angiopep-2 conjugates and fusion proteins which can be used to treat MPS-II. Because these conjugates and fusion proteins are capable of crossing the BBB, they can treat not only the peripheral disease symptoms, but may also be effective in treating CNS symptoms. In addition, because targeting moieties such as Angiopep-2 are capable of targeting enzymes to the lysosomes, it is expected that these conjugates and fusion proteins are more effective than the enzymes by themselves.

Accordingly, in a first aspect, the invention features a compound including (a) a targeting moiety (e.g., a peptide or peptidomimetic targeting moiety that may be less than 200, 150, 125, 100, 80, 60, 50, 40, 35, 30, 25, 24, 23, 22, 21, 20, or 19 amino acids) and (b) a lysosomal enzyme, an active fragment thereof, or an analog thereof, where the targeting moiety and the enzyme, fragment, or analog are joined by a linker, wherein the targeting moiety is capable of transporting said enzyme, fragment or analog to the lysosome and/or across the blood brain barrier, wherein the compound exhibits IDS enzymatic activity, wherein the linker joining the enzyme and the peptide targeting moiety can be formed by a click chemistry reaction between a click chemistry pair and wherein the linker does not have the structure:

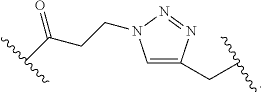 [SMILES: *Cc1cn(CCC(*)=O)nn1]

In a more particular aspect, the invention features a compound comprising: (a) a peptide or peptidomimetic targeting moiety less than 150 amino acids and (b) an enzyme selected from the group consisting of iduronate-2-sulfatase (IDS), an IDS fragment having IDS activity, or an IDS analog having IDS activity, wherein the targeting moiety is capable of transporting said enzyme, fragment or analog to the lysosome and/or across the blood brain barrier, wherein the targeting moiety and the enzyme are joined by a linker selected from the group consisting of a monofluorocyclooctyne (MFCO) containing linker, a difluorocyclooctyne (DFCO) containing linker, a cyclooctyne (OCT) containing linker, a dibenzocyclooctyne (DIBO) containing linker, a biarylazacyclooctyne (BARAC) containing linker, a difluorobenzocyclooctyne (DIFBO) containing linker, and a bicyclo[6.1.0]nonyne (BCN).

The lysosomal enzyme may be iduronate-2-sulfatase (IDS), an IDS fragment having IDS activity, or an IDS analog. In certain embodiments, the IDS enzyme or the IDS fragment has the amino acid sequence of human IDS isoform a or a fragment thereof (e.g., amino acids 26-550 of isoform a) or the IDS analog is substantially identical (e.g., at least 60%, 70%, 80%, 85%, 90%, 95%, 96%, 97%, 98%, or 99% identical) to the sequence of human IDS isoform a, isoform b, isoform c, or to amino acids 26-550 of isoform a. In a particular embodiment, the IDS enzyme has the sequence of human IDS isoform a or the mature form of isoform a (amino acids 26-550 of isoform a).

In the first aspect, the targeting moiety may include an amino acid sequence that is substantially identical to any of SEQ ID NOS:1-105 or 107-117 (e.g., Angiopep-2 (SEQ ID NO:97)). In other embodiments, the targeting moiety includes the formula Lys-Arg-X3-X4-X5-Lys (formula Ia), where X3 is Asn or Gln; X4 is Asn or Gln; and X5 is Phe, Tyr, or Trp, where the targeting moiety optionally includes one or more D-isomers of an amino acid recited in formula Ia. In other embodiments, the targeting moiety includes the formula Z1-Lys-Arg-X3-X4-X5-Lys-Z2 (formula Ib), where X3 is Asn or Gln; X4 is Asn or Gln; X5 is Phe, Tyr, or Trp; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; and where the targeting moiety optionally includes one or more D-isomers of an amino acid recited in formula Ib, Z1, or Z2. In other embodiments, the targeting moiety includes the formula X1-X2-Asn-Asn-X5-X6 (formula IIa), where X1 is Lys or D-Lys; X2 is Arg or D-Arg; X5 is Phe or D-Phe; and X6 is Lys or D-Lys; and where at least one of X1, X2, X5, or X6 is a D-amino acid. In other embodiments, the targeting moiety includes the formula X1-X2-Asn-Asn-X5-X6-X7 (formula IIb), where X1 is Lys or D-Lys; X2 is Arg or D-Arg; X5 is Phe or D-Phe; X6 is Lys or D-Lys; and X7 is Tyr or D-Tyr; and where at least one of X1, X2, X5, X6, or X7 is a D-amino acid. In other embodiments, the targeting moiety includes the formula Z1-X1-X2-Asn-Asn-X5-X6-X7-Z2 (formula IIc), where X1 is Lys or D-Lys; X2 is Arg or D-Arg; X5 is Phe or D-Phe; X6 is Lys or D-Lys; X7 is Tyr or D-Tyr; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; where at least one of X1, X2, X5, X6, or X7 is a D-amino acid; and where the peptide or peptidomimetic optionally includes one or more D-isomers of an amino acid recited in Z1 or Z2.

In the first aspect, the linker may be a covalent bond (e.g., a peptide bond) or one or more amino acids. The compound may be a fusion protein (e.g., Angiopep-2-IDS, IDS-Angiopep-2, or Angiopep-2-IDS-Angiopep-2, or has the structure shown in FIG. 1). The compound may further include a second targeting moiety that is joined to the compound by a second linker.

The invention also features a pharmaceutical composition including a compound of the first aspect and a pharmaceutically acceptable carrier.

In another aspect, the invention features a method of treating or treating prophylactically a subject having a lysosomal storage disorder (e.g., MPS-II). The method includes administering to the subject a compound of the first aspect or a pharmaceutical composition described herein. The lysosomal enzyme in the compound may be IDS. The subject may have either the severe form of MPS-II or the attenuated form of MPS-II. The subject may be experiencing neurological symptoms (e.g., mental retardation). The method may be performed on or started on a subject that is less than six months, or 1, 2, 3, 4, 5, 6, 7, 8, 9, 10, 11, 12, 13, 15, or 18 years of age. The subject may be an infant (e.g., less than 1 year old).

In certain embodiments, the targeting moiety is not an antibody (e.g., an antibody or an immunoglobulin that is specific for an endogenous BBB receptor such as the insulin receptor, the transferrin receptor, the leptin receptor, the lipoprotein receptor, and the IGF receptor).

In any of the above aspects, the targeting moiety may be substantially identical to any of the sequences of Table 1, or a fragment thereof. In certain embodiments, the peptide has a sequence of Angiopep-1 (SEQ ID NO:67), Angiopep-2 (SEQ ID NO:97) (An2), Angiopep-3 (SEQ ID NO:107), Angiopep-4a (SEQ ID NO:108), Angiopep-4b (SEQ ID NO:109), Angiopep-5 (SEQ ID NO:110), Angiopep-6 (SEQ ID NO:111), Angiopep-7 (SEQ ID NO:112)) or reversed Angiopep-2 (SEQ ID NO:117). The targeting moiety or compound may be efficiently transported into a particular cell type (e.g., any one, two, three, four, or five of liver, lung, kidney, spleen, and muscle) or may cross the mammalian BBB efficiently (e.g., Angiopep-1, -2, -3, -4a, -4b, -5, and -6). In another embodiment, the targeting moiety or compound is able to enter a particular cell type (e.g., any one, two, three, four, or five of liver, lung, kidney, spleen, and muscle) but does not cross the BBB efficiently (e.g., a conjugate including Angiopep-7). The targeting moiety may be of any length, for example, at least 6, 7, 8, 9, 10, 11, 12, 13, 14, 15, 16, 17, 18, 19, 20, 21, 25, 35, 50, 75, 100, 200, or 500 amino acids, or any range between these numbers. In certain embodiments, the targeting moiety is less than 200, 150, 125, 100, 90, 80, 70, 60, 50, 40, 30, 25, 24, 23, 22, 21, 20, 19, 18, 17, 16, 15, 14, 13, 12, 11, 10, 9, 8, 7, or 6 amino acids (e.g., 10 to 50 amino acids in length). The targeting moiety may be produced by recombinant genetic technology or chemical synthesis.

TABLE 1
Exemplary targeting moieties

| SEQ ID | |
|---|---|
| NO: | |
| 1 | T F V Y G G C R A K R N N F K S A E D |
| 2 | T F Q Y G G C M G N G N N F V T E K E |
| 3 | P F F Y G G C G G N R N N F D T E E Y |
| 4 | S F Y Y G G C L G N K N N Y L R E E E |
| 5 | T F F Y G G C R A K R N N F K R A K Y |
| 6 | T F F Y G G C R G K R N N F K R A K Y |
| 7 | T F F Y G G C R A K K N N Y K R A K Y |
| 8 | T F F Y G G C R G K K N N F K R A K Y |
| 9 | T F Q Y G G C R A K R N N F K R A K Y |
| 10 | T F Q Y G G C R G K K N N F K R A K Y |
| 11 | T F F Y G G C L G K R N N F K R A K Y |
| 12 | T F F Y G G S L G K R N N F K R A K Y |
| 13 | P F F Y G G C G G K K N N F K R A K Y |
| 14 | T F F Y G G C R G K G N N Y K R A K Y |
| 15 | P F F Y G G C R G K R N N F L R A K Y |
| 16 | T F F Y G G C R G K R N N F K R E K Y |
| 17 | P F F Y G G C R A K K N N F K R A K E |
| 18 | T F F Y G G C R G K R N N F K R A K D |
| 19 | T F F Y G G C R A K R N N F D R A K Y |
| 20 | T F F Y G G C R G K K N N F K R A E Y |
| 21 | P F F Y G G C G A N R N N F K R A K Y |
| 22 | T F F Y G G C G G K K N N F K T A K Y |
| 23 | T F F Y G G C R G N R N N F L R A K Y |
| 24 | T F F Y G G C R G N R N N F K T A K Y |
| 25 | T F F Y G G S R G N R N N F K T A K Y |
| 26 | T F F Y G G C L G N G N N F K R A K Y |
| 27 | T F F Y G G C L G N R N N F L R A K Y |
| 28 | T F F Y G G C L G N R N N F K T A K Y |
| 29 | T F F Y G G C R G N G N N F K S A K Y |
| 30 | T F F Y G G C R G K K N N F D R E K Y |
| 31 | T F F Y G G C R G K R N N F L R E K E |
| 32 | T F F Y G G C R G K G N N F D R A K Y |
| 33 | T F F Y G G S R G K G N N F D R A K Y |
| 34 | T F F Y G G C R G N G N N F V T A K Y |
| 35 | P F F Y G G C G G K G N N Y V T A K Y |
| 36 | T F F Y G G C L G K G N N F L T A K Y |
| 37 | S F F Y G G C L G N K N N F L T A K Y |
| 38 | T F F Y G G C G G N K N N F V R E K Y |
| 39 | T F F Y G G C M G N K N N F V R E K Y |
| 40 | T F F Y G G S M G N K N N F V R E K Y |
| 41 | P F F Y G G C L G N R N N Y V R E K Y |
| 42 | T F F Y G G C L G N R N N F V R E K Y |
| 43 | T F F Y G G C L G N K N N Y V R E K Y |
| 44 | T F F Y G G C G G N G N N F L T A K Y |
| 45 | T F F Y G G C R G N R N N F L T A E Y |
| 46 | T F F Y G G C R G N G N N F K S A E Y |
| 47 | P F F Y G G C L G N K N N F K T A E Y |
| 48 | T F F Y G G C R G N R N N F K T E E Y |
| 49 | T F F Y G G C R G K R N N F K T E E D |
| 50 | P F F Y G G C G G N G N N F V R E K Y |
| 51 | S F F Y G G C M G N G N N F V R E K Y |
| 52 | P F F Y G G C G G N G N N F L R E K Y |
| 53 | T F F Y G G C L G N G N N F V R E K Y |
| 54 | S F F Y G G C L G N G N N Y L R E K Y |
| 55 | T F F Y G G S L G N G N N F V R E K Y |
| 56 | T F F Y G G C R G N G N N F V T A E Y |
| 57 | T F F Y G G C L G K G N N F V S A E Y |
| 58 | T F F Y G G C L G N R N N F D R A E Y |
| 59 | T F F Y G G C L G N R N N F L R E E Y |
| 60 | T F F Y G G C L G N K N N Y L R E E Y |
| 61 | P F F Y G G C G G N R N N Y L R E E Y |
| 62 | P F F Y G G S G G N R N N Y L R E E Y |
| 63 | M R P D F C L E P P Y T G P C V A R I |
| 64 | A R I I R Y F Y N A K A G L C Q T F V Y G |
| 65 | Y G G C R A K R N N Y K S A E D C M R T C G |
| 66 | P D F C L E P P Y T G P C V A R I I R Y F Y |
| 67 | T F F Y G G C R G K R N N F K T E E Y |
| 68 | K F F Y G G C R G K R N N F K T E E Y |
| 69 | T F Y Y G G C R G K R N N Y K T E E Y |
| 70 | T F F Y G G S R G K R N N F K T E E Y |
| 71 | C T F F Y G C C R G K R N N F K T E E Y |
| 72 | T F F Y G G C R G K R N N F K T E E Y C |
| 73 | C T F F Y G S C R G K R N N F K T E E Y |
| 74 | T F F Y G G S R G K R N N F K T E E Y C |
| 75 | P F F Y G G C R G K R N N F K T E E Y |
| 76 | T F F Y G G C R G K R N N F K T K E Y |
| 77 | T F F Y G G K R G K R N N F K T E E Y |
| 78 | T F F Y G G C R G K R N N F K T K R Y |
| 79 | T F F Y G G K R G K R N N F K T A E Y |
| 80 | T F F Y G G K R G K R N N F K T A G Y |
| 81 | T F F Y G G K R G K R N N F K R E K Y |
| 82 | T F F Y G G K R G K R N N F K R A K Y |
| 83 | T F F Y G G C L G N R N N F K T E E Y |
| 84 | T F F Y G C G R G K R N N F K T E E Y |
| 85 | T F F Y G G R C G K R N N F K T E E Y |
| 86 | T F F Y G G C L G N G N N F D T E E E |
| 87 | T F Q Y G G C R G K R N N F K T E E Y |
| 88 | Y N K E F G T F N T K G C E R G Y R F |
| 89 | R F K Y G G C L G N M N N F E T L E E |
| 90 | R F K Y G G C L G N K N N F L R L K Y |
| 91 | R F K Y G G C L G N K N N Y L R L K Y |
| 92 | K T K R K R K K Q R V K I A Y E E I F K N Y |
| 93 | K T K R K R K K Q R V K I A Y |
| 94 | R G G R L S Y S R R F S T S T G R |
| 95 | R R L S Y S R R R F |
| 96 | R Q I K I W F Q N R R M K W K K |
| 97 | T F F Y G G S R G K R N N F K T E E Y |
| 98 | M R P D F C L E P P Y T G P C V A R I |
| | I R Y F Y N A K A G L C Q T F V Y G G |
| | C R A K R N N F K S A E D C M R T C G G A |
| 99 | T F F Y G G C R G K R N N F K T K E Y |
| 100 | R F K Y G G C L G N K N N Y L R L K Y |
| 101 | T F F Y G G C R A K R N N F K R A K Y |
| 102 | N A K A G L C Q T F V Y G G C L A K R N N F |
| | E S A E D C M R T C G G A |
| 103 | Y G G C R A K R N N F K S A E D C M R T C G |
| | G A |
| 104 | G L C Q T F V Y G G C R A K R N N F K S A E |
| 105 | L C Q T F V Y G G C E A K R N N F K S A |
| 107 | T F F Y G G S R G K R N N F K T E E Y |
| 108 | R F F Y G G S R G K R N N F K T E E Y |
| 109 | R F F Y G G S R G K R N N F K T E E Y |
| 110 | R F F Y G G S R G K R N N F R T E E Y |
| 111 | T F F Y G G S R G K R N N F R T E E Y |
| 112 | T F F Y G G S R G R R N N F R T E E Y |
| 113 | C T F F Y G G S R G K R N N F K T E E Y |
| 114 | T F F Y G G S R G K R N N F K T E E Y C |
| 115 | C T F F Y G G S R G R R N N F R T E E Y |
| 116 | T F F Y G G S R G R R N N F R T E E Y C |
| 117 | Y E E T K F N N R K G R S G G Y F F T |

Polypeptides Nos. 5, 67, 76, and 91, include the sequences of SEQ ID NOS: 5, 67, 76, and 91, respectively, and are amidated at the C-terminus
Polypeptides Nos. 107, 109, and 110 include the sequences of SEQ ID NOS: 97, 109, and 110, respectively, and are acetylated at the N-terminus

In any of the above aspects, the targeting moiety may include an amino acid sequence having the formula:

X1-X2-X3-X4-X5-X6-X7-X8-X9-X10-X11-X12-X13-X14-X15-X16-X17-X18-X19

where each of X1-X19 (e.g., X1-X6, X8, X9, X11-X14, and X16-X19) is, independently, any amino acid (e.g., a naturally occurring amino acid such as Ala, Arg, Asn, Asp, Cys, Gln, Glu, Gly, His, Ile, Leu, Lys, Met, Phe, Pro, Ser, Thr, Trp, Tyr, and Val) or absent and at least one (e.g., 2 or 3) of X1, X10, and X15 is arginine. In some embodiments, X7 is Ser or Cys; or X10 and X15 each are independently Arg or Lys. In some embodiments, the residues from X1 through X19, inclusive, are substantially identical to any of the amino acid sequences of any one of SEQ ID NOS:1-105 and 107-116 (e.g., Angiopep-1, Angiopep-2, Angiopep-3, Angiopep-4a, Angiopep-4b, Angiopep-5, Angiopep-6, and Angiopep-7). In some embodiments, at least one (e.g., 2, 3, 4, or 5) of the amino acids X1-X19 is Arg. In some embodiments, the peptide has one or more additional cysteine residues at the N-terminal of the peptide, the C-terminal of the peptide, or both.

In any of the above aspects, the targeting moiety may include the amino acid sequence Lys-Arg-X3-X4-X5-Lys (formula Ia), where X3 is Asn or Gln; X4 is Asn or Gln; and X5 is Phe, Tyr, or Trp; where the peptide is optionally fewer than 200 amino acids in length (e.g., fewer than 150, 100, 75, 50, 45, 40, 35, 30, 25, 20, 19, 18, 17, 16, 15, 14, 12, 10, 11, 8, or 7 amino acids, or any range between these numbers); where the peptide or peptidomimetic optionally includes one or more D-isomers of an amino acid recited in formula Ia (e.g., a D-isomer of Lys, Arg, X3, X4, X5, or Lys); and where the peptide or peptidomimetic is not a peptide in Table 2.

In any of the above aspects, the targeting moiety may include the amino acid sequence Lys-Arg-X3-X4-X5-Lys (formula Ia), where X3 is Asn or Gln; X4 is Asn or Gln; and X5 is Phe, Tyr, or Trp; where the peptide or peptidomimetic is fewer than 19 amino acids in length (e.g., fewer than 18, 17, 16, 15, 14, 12, 10, 11, 8, or 7 amino acids, or any range between these numbers); and where the peptide or peptidomimetic optionally includes one or more D-isomers of an amino acid recited in formula Ia (e.g., a D-isomer of Lys, Arg, X3, X4, X5, or Lys).

In any of the above aspects, the targeting moiety may include the amino acid sequence of Z1-Lys-Arg-X3-X4-X5-Lys-Z2 (formula Ib), where X3 is Asn or Gln; X4 is Asn or Gln; X5 is Phe, Tyr, or Trp; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; and where the peptide or peptidomimetic optionally comprises one or more D-isomers of an amino acid recited in formula Ib, Z1, or Z2.

In any of the above aspects, the targeting moiety may include the amino acid sequence Lys-Arg-Asn-Asn-Phe-Lys. In other embodiments, the targeting moiety has an amino acid sequence of Lys-Arg-Asn-Asn-Phe-Lys-Tyr. In still other embodiments, the targeting moiety has an amino acid sequence of Lys-Arg-Asn-Asn-Phe-Lys-Tyr-Cys.

In any of the above aspects, the targeting moiety may have the amino acid sequence of X1-X2-Asn-Asn-X5-X6 (formula IIa), where X1 is Lys or D-Lys; X2 is Arg or D-Arg; X5 is Phe or D-Phe; and X6 is Lys or D-Lys; and where at least one (e.g., at least two, three, or four) of X1, X2, X5, or X6 is a D-amino acid.

In any of the above aspects, the targeting moiety may have the amino acid sequence of X1-X2-Asn-Asn-X5-X6-X7 (formula IIb), where X1 is Lys or D-Lys; X2 is Arg or D-Arg; X5 is Phe or D-Phe; X6 is Lys or D-Lys; and X7 is Tyr or D-Tyr; and where at least one (e.g., at least two, three, four, or five) of X1, X2, X5, X6, or X7 is a D-amino acid.

In any of the above aspects, the targeting moiety may have the amino acid sequence of Z1-Lys-Arg-X3-X4-X5-Lys-Z2 (formula IIc), where X3 is Asn or Gln; X4 is Asn or Gln; X5 is Phe, Tyr, or Trp; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; where at least one of X1, X2, X5, X6, or X7 is a D-amino acid; and where the peptide or peptidomimetic optionally comprises one or more D-isomers of an amino acid recited in Z1 or Z2.

In any of the above aspects, the targeting moiety may have the amino acid sequence of Thr-Phe-Phe-Tyr-Gly-Gly-Ser-Arg-Gly-Lys-Arg-Asn-Asn-Phe-Lys-Thr-Glu-Glu-Tyr (An2), where any one or more amino acids are D-isomers. For example, the targeting moiety can have 1, 2, 3, 4, or 5 amino acids which are D-isomers. In a preferred embodiment, one or more or all of positions 8, 10, and 11 can be D-isomers. In yet another embodiment, one or more or all of positions 8, 10, 11, and 15 can have D-isomers.

In any of the above aspects, the targeting moiety may be Thr-Phe-Phe-Tyr-Gly-Gly-Ser-D-Arg-Gly-D-Lys-D-Arg-Asn-Asn-Phe-Lys-Thr-Glu-Glu-Tyr (3D-An2); Phe-Tyr-Gly-Gly-Ser-Arg-Gly-Lys-Arg-Asn-Asn-Phe-Lys-Thr-Glu-Glu-Tyr-Cys (P1); Phe-Tyr-Gly-Gly-Ser-Arg-Gly-D-Lys-D-Arg-Asn-Asn-D-Phe-Lys-Thr-Glu-Glu-Tyr-Cys (P1a); Phe-Tyr-Gly-Gly-Ser-Arg-Gly-D-Lys-D-Arg-Asn-Asn-D-Phe-D-Lys-Thr-Glu-Glu-Tyr-Cys (P1b); Phe-Tyr-Gly-Gly-Ser-Arg-Gly-D-Lys-D-Arg-Asn-Asn-D-Phe-D-Lys-Thr-Glu-Glu-D-Tyr-Cys (P1c); D-Phe-D-Tyr-Gly-Gly-Ser-D-Arg-Gly-D-Lys-D-Arg-Asn-Asn-D-Phe-D-Lys-Thr-Glu-D-Glu-D-Tyr-Cys (P1d); Gly-Gly-Ser-Arg-Gly-Lys-Arg-Asn-Asn-Phe-Lys-Thr-Glu-Glu-Tyr-Cys (P2); Ser-Arg-Gly-Lys-Arg-Asn-Asn-Phe-Lys-Thr-Glu-Glu-Tyr-Cys (P3); Gly-Lys-Arg-Asn-Asn-Phe-Lys-Thr-Glu-Glu-Tyr-Cys (P4); Lys-Arg-Asn-Asn-Phe-Lys-Thr-Glu-Glu-Tyr-Cys (P5); D-Lys-D-Arg-Asn-Asn-D-Phe-Lys-Thr-Glu-Glu-Tyr-Cys (P5a); D-Lys-D-Arg-Asn-Asn-D-Phe-D-Lys-Thr-Glu-Glu-Tyr-Cys (P5b); D-Lys-D-Arg-Asn-Asn-D-Phe-D-Lys-Thr-Glu-Glu-D-Tyr-Cys (P5c); Lys-Arg-Asn-Asn-Phe-Lys-Tyr-Cys (P6); D-Lys-D-Arg-Asn-Asn-D-Phe-Lys-Tyr-Cys (P6a); D-Lys-D-Arg-Asn-Asn-D-Phe-D-Lys-Tyr-Cys (P6b); Thr-Phe-Phe-Tyr-Gly-Gly-Ser-D-Arg-Gly-D-Lys-D-Arg-Asn-Asn-Phe-D-Lys-Thr-Glu-Glu-Tyr; and D-Lys-D-Arg-Asn-Asn-D-Phe-D-Lys-D-Tyr-Cys (P6c); or a fragment thereof. In other embodiments, the targeting moiety has a sequence of one of the aforementioned peptides or peptidomimetics having from 0 to 5 (e.g., from 0 to 4, 0 to 3, 0 to 2, 0 to 1, 1 to 5, 1 to 4, 1 to 3, 1 to 2, 2 to 5, 2 to 4, 2 to 3, 3 to 5, 3 to 4, or 4 to 5) substitutions, deletions, or additions of amino acids.

In any of the above aspects, the peptide may be Phe-Tyr-Gly-Gly-Ser-Arg-Gly-Lys-Arg-Asn-Asn-Phe-Lys-Thr-Glu-Glu; Gly-Gly-Ser-Arg-Gly-Lys-Arg-Asn-Asn-Phe-Lys-Thr-Glu-Glu; Ser-Arg-Gly-Lys-Arg-Asn-Asn-Phe-Lys-Thr-Glu-Glu; Gly-Lys-Arg-Asn-Asn-Phe-Lys-Thr-Glu-Glu; Lys-Arg-Asn-Asn-Phe-Lys-Thr-Glu-Glu; or Lys-Arg-Asn-Asn-Phe-Lys, or a fragment thereof.

In any of the above aspects, the peptidomimetic may be Thr-Phe-Phe-Tyr-Gly-Gly-Ser-D-Arg-Gly-D-Lys-D-Arg-Asn-Asn-Phe-Lys-Thr-Glu-Glu-Tyr (3D-An2); Phe-Tyr-Gly-Gly-Ser-Arg-Gly-Lys-Arg-Asn-Asn-Phe-Lys-Thr-Glu-Glu-Tyr-Cys (P1); Phe-Tyr-Gly-Gly-Ser-Arg-Gly-D-Lys-D-Arg-Asn-Asn-D-Phe-Lys-Thr-Glu-Glu-Tyr-Cys (P1a); Phe-Tyr-Gly-Gly-Ser-Arg-Gly-D-Lys-D-Arg-Asn-Asn-D-Phe-D-Lys-Thr-Glu-Glu-Tyr-Cys (P1b); Phe-Tyr-Gly-Gly-Ser-Arg-Gly-D-Lys-D-Arg-Asn-Asn-D-Phe-D-Lys-Thr-Glu-Glu-D-Tyr-Cys (P1c); D-Phe-D-Tyr-Gly-Gly-Ser-D-Arg-Gly-D-Lys-D-Arg-Asn-Asn-D-Phe-D-Lys-Thr-Glu-D-Glu-D-Tyr-Cys (P1d) or a fragment thereof (e.g., deletion of 1 to 7 amino acids from the N-terminus of P1, P1a, P1b, P1c, or P1 d; a deletion of 1 to 5 amino acids from the C-terminus of P1, P1a, P1b, P1c, or P1 d; or deletions of 1 to 7 amino acids from the N-terminus of P1, P1a, P1b, P1c, or P1d and 1 to 5 amino acids from the C-terminus of P1, P1a, P1b, P1c, or P1d).

In any of the targeting moieties described herein, the moiety may include additions or deletions of 1, 2, 3, 4, or 5 amino acids (e.g., from 1 to 3 amino acids) may be made from an amino acid sequence described herein (e.g., from Lys-Arg-X3-X4-X5-Lys).

In any of the targeting moieties described herein, the moiety may have one or more additional cysteine residues at the N-terminal of the peptide or peptidomimetic, the C-terminal of the peptide or peptidomimetic, or both. In other embodiments, the targeting moiety may have one or more additional tyrosine residues at the N-terminal of the peptide or peptidomimetic, the C-terminal of the peptide or peptidomimetic, or both. In yet further embodiments, the targeting moiety has the amino acid sequence Tyr-Cys and/or Cys-Tyr at the N-terminal of the peptide or peptidomimetic, the C-terminal of the peptide or peptidomimetic, or both.

In certain embodiments of any of the above aspects, the targeting moiety may be fewer than 15 amino acids in length (e.g., fewer than 10 amino acids in length).

In certain embodiments of any of the above aspects, the targeting moiety may have a C-terminus that is amidated. In other embodiments, the targeting moiety is efficiently transported across the BBB (e.g., is transported across the BBB more efficiently than Angiopep-2).

In certain embodiments of any of the above aspects, the fusion protein, targeting moiety, or lysosomal enzyme (e.g., IDS) or fragment is modified to be an analog or peptidomimetic (e.g., as described herein). The fusion protein, targeting moiety, or lysosomal enzyme, fragment, or analog may be amidated, acetylated, or both. Such modifications may be at the amino or carboxy terminus of the peptide or enzyme. The fusion protein, targeting moiety, or lysosomal enzyme, fragment, or analog may be in a multimeric form, for example, dimeric form (e.g., formed by disulfide bonding through cysteine residues).

In certain embodiments, the targeting moiety, lysosomal enzyme (e.g., IDS), enzyme fragment, or enzyme analog has an amino acid sequence described herein with at least one amino acid substitution (e.g., 2, 3, 4, 5, 6, 7, 8, 9, 10, 11, or 12 substitutions), insertion, or deletion. The peptide may contain, for example, 1 to 12, 1 to 10, 1 to 5, or 1 to 3 amino acid substitutions, for example, 1 to 10 (e.g., to 9, 8, 7, 6, 5, 4, 3, 2) amino acid substitutions. The amino acid substitution(s) may be conservative or non-conservative. For example, the targeting moiety may have an arginine at one, two, or three of the positions corresponding to positions 1, 10, and 15 of the amino acid sequence of any of SEQ ID NO:1, Angiopep-1, Angiopep-2, Angiopep-3, Angiopep-4a, Angiopep-4b, Angiopep-5, Angiopep-6, and Angiopep-7.

In any of the above aspects, the compound may specifically exclude a peptide including or consisting of any of SEQ ID NOS:1-105 and 107-117 (e.g., Angiopep-1, Angiopep-2, Angiopep-3, Angiopep-4a, Angiopep-4b, Angiopep-5, Angiopep-6, and Angiopep-7). In some embodiments, the peptides of the invention exclude the peptides of SEQ ID NOS:102, 103, 104, and 105.

In any of the above aspects, the linker (X) may be any linker known in the art or described herein. In particular embodiments, the linker is a covalent bond (e.g., a peptide bond), a chemical linking agent (e.g., those described herein), an amino acid or a peptide (e.g., 2, 3, 4, 5, 8, 10, or more amino acids). For the avoidance of doubt, a chemical linking agent may include one or more amino acids in addition to non-amino acid portions.

In certain embodiments, the linker has the formula:

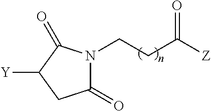

where n is an integer between 2 and 15 (e.g., 2, 3, 4, 5, 6, 7, 8, 9, 10, 11, 12, 13, 14, or 15); and either Y is a thiol on A and Z is a primary amine on B or Y is a thiol on B and Z is a primary amine on A. In certain embodiments, the linker is an N-Succinimidyl (acetylthio)acetate (SATA) linker or a hydrazide linker. The linker may be conjugated to the enzyme (e.g., IDS) or the targeting moiety (e.g., Angiopep-2), through a free amine, a cysteine side chain (e.g., of Angiopep-2-Cys or Cys-Angiopep-2), or through a glycosylation site.

In certain embodiments, the compound has the formula

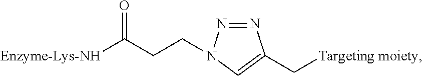

where the “Lys-NH” group represents either a lysine present in the enzyme or an N-terminal or C-terminal lysine. In another example, the compound has the structure:

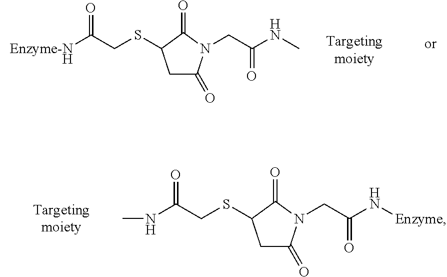

where each —NH— group represents a primary amino present on the targeting moiety and the enzyme, respectively. In particular embodiments, the enzyme may be IDS or the targeting moiety may be Angiopep-2.

In certain embodiments, the compound is a fusion protein including the targeting moiety (e.g., Angiopep-2) and the lysosomal enzyme (e.g., IDS), enzyme fragment, or enzyme analog.

In certain embodiments, the linker is formed by the reaction of a click-chemistry reaction pair where the click chemistry reaction is selected from the group consisting of a Huisgen 1,3-dipolar cycloaddition reaction between an alkynyl group and an azido group to form a triazole-containing linker; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; and a splint ligation reaction with a phosphorothioate group and an iodo group; and a reductive amination reaction with an aldehyde group and an amino group. In one aspect of the invention, the click chemistry reaction is a Huisgen 1,3-dipolar cycloaddition reaction between an alkynyl group and an azido group. In such embodiments, the alknyl group comprises monofluorocyclooctyne (MFCO), difluorocyclooctyne (DFCO), cyclooctyne (OCT), dibenzocyclooctyne (DIBO), biarylazacyclooctyne (BARAC), difluorobenzocyclooctyne (DIFBO), or bicyclo[6.1.0]nonyne (BCN).

In certain embodiments, the targeting moiety can be derivatized with an azide group at the N- or C-terminus of the polypeptide, such that the azide group can be reacted with an alkyne derivatized linker, in a click-chemistry reaction, to attach the targeting moiety to the linker. In more particular embodiments, Angiopep-2 can be derivatized with an azide group at the N- or C-terminus of the polypeptide (optionally on an amino acid side chain at the N- or C-terminus), such that the azide group can be reacted with an alkyne derivatized linker attached to the enzyme, in a click-chemistry reaction, to attach the Angiopep-2 to the linker and enzyme.

In another aspect, the linker is a maleimide group or an S-acetylthioacetate (SATA) group.

In one embodiment, the compound includes an Angiopep-2 joined to the enzyme (e.g., IDS, an active IDS fragment, or an IDS analog) via a BCN containing linker. This compound can have the general structure

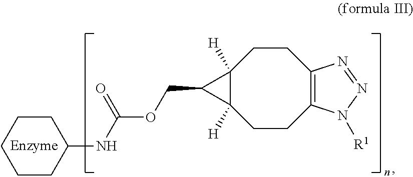

wherein R¹ is:

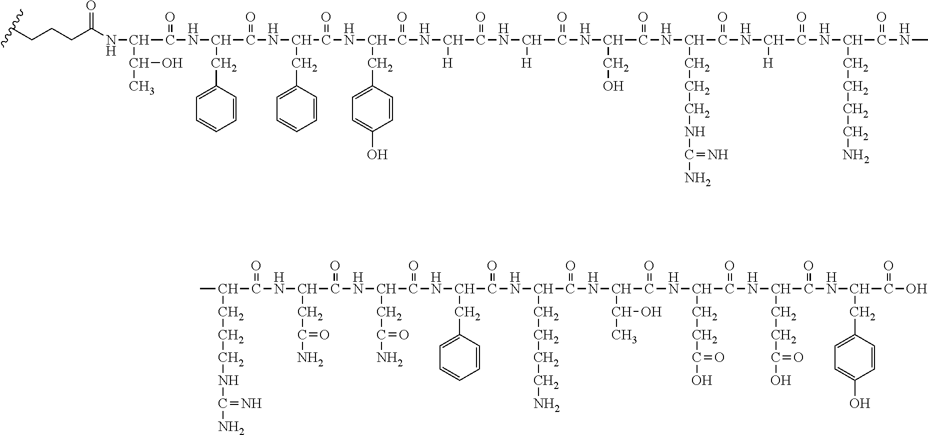

where n is 1 to 6 and the NH group attached to the enzyme is derived from the reaction of a primary amino group in the enzyme. When n is 1, the compound has the structure:

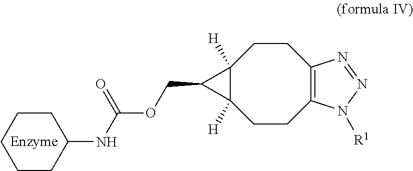

When n is 2, the compound has the structure:

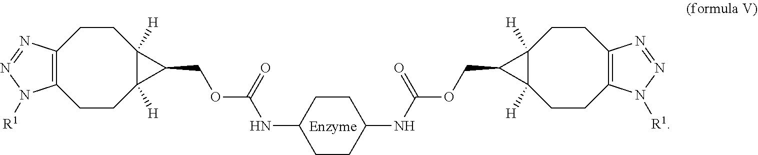

In the compounds of formula IV and V, the NH group(s) attached to enzyme is (are) derived from the reaction of a primary amino group in the enzyme and R¹ is:

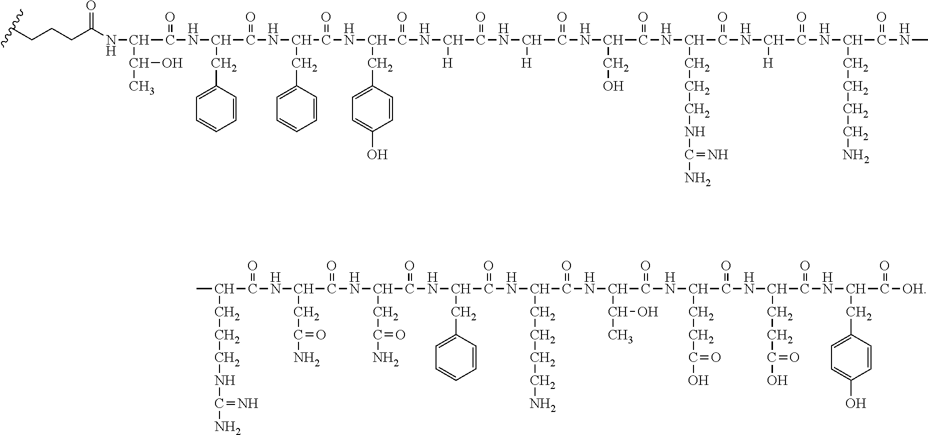

The invention also features a population of compounds of formula III where the average value of n is between 1 and 6 (e.g., 1, 1.2, 1.5, 2, 2.4, 2.5, 2.8, 3, 3.5, 4, 4.5, 5, 5.5, or 6). More particularly, the invention features a population of compounds of formula III where the average value of n is between 1 and 4. Even more particularly, the invention features a population of compounds of formula III where the average value of n is about 1, about 2.4 or about 3.

In certain embodiments, one or more NH groups attached to enzyme in the compound of formula III are derived from the primary amino groups of one or more lysine residues. In further embodiments of compound III, one or more NH groups attached to enzyme are derived from one or more primary amino groups of lysine 199 and/or lysine 376 (using the numbering of full length human IDS isoform a).

The compound with a BCN containing linker can also have the structure

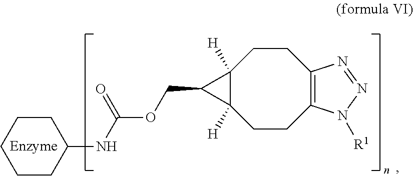

wherein R¹ is:

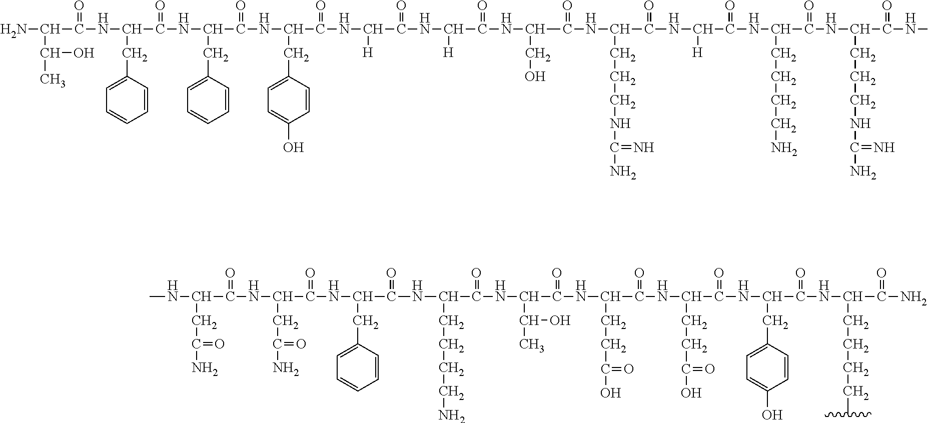

where enzyme represents IDS, an active fragment of IDS or an active analog of IDS, where n is an integer between 1 and 6 and where the NH group attached to the enzyme is derived from the reaction of a primary amino group in the enzyme.

The invention features a population of compounds of formula VI where the average value of n is between 1 and 6 (e.g., 1, 1.5, 2, 2.3, 2.5, 3, 3.5, 4, 4.5, 5, 5.5, or 6). More particularly, the invention features a population of compounds of formula VI where the average value of n is about 2.3.

In certain embodiments, one or more NH group(s) attached to enzyme in the compound of formula VI are derived from one or more primary amino groups of lysine residues. In further embodiments of compound VI, one or more NH group(s) attached to enzyme are derived from one or more primary amino groups of lysine 199 and/or lysine 479 (using the numbering of full length human IDS isoform a).

In one embodiment, the compound includes an Angiopep-2 joined to IDS, an active IDS fragment or an active IDS analog via an MFCO containing linker. The compound can have the general structure:

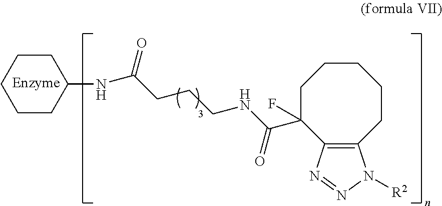

wherein R² is:

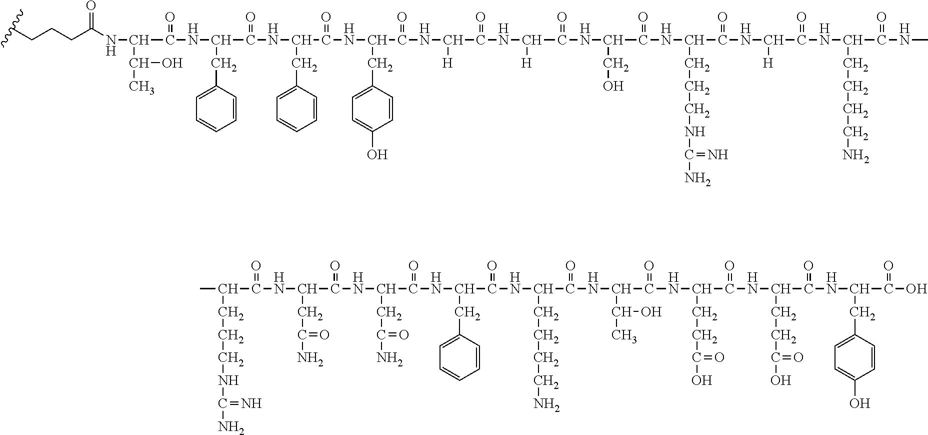

where enzyme represents IDS, an active fragment of IDS or an active analog of IDS, where n is an integer between 1 and 6 and where the NH group attached to the enzyme is derived from the reaction of a primary amino group in the enzyme.

The invention also features a population of compounds of formula VII where the average value of n is between 1 and 6 (e.g., 1, 1.5, 2, 2.3, 2.5, 2.6, 3, 3.5, 4, 4.2, 4.4, 4.5, 5, 5.3, 5.5, or 6). More particularly, the invention features a population of compounds of formula VII where the average value of n is about 2.3, about 4.4, or about 5.0.

The compound with an MFCO containing linker can also have the structure

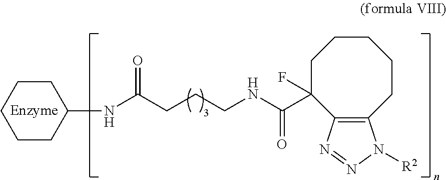

wherein R² is:

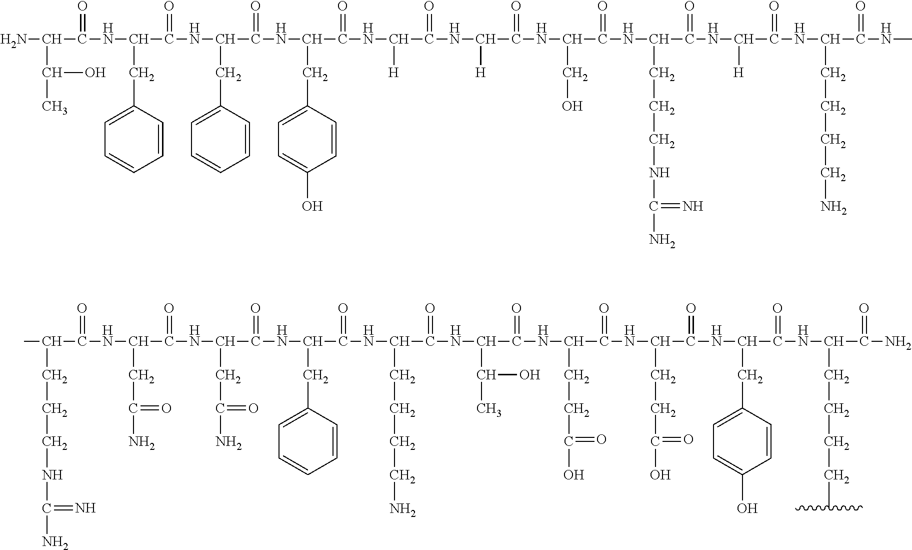

where enzyme represents IDS, an active fragment of IDS or an active analog of IDS, where n is an integer between 1 and 6 and where the NH group attached to the enzyme is derived from the reaction of a primary amino group in the enzyme.

The invention features a population of compounds of formula VIII where the average value of n is between 1 and 6 (e.g., 1, 1.5, 2, 2.5, 3, 3.5, 4, 4.5, 4.8, 4.9, 5, 5.5, or 6). More particularly, the invention features a population of compounds of formula VIII where the average value of n is about 4.9.

In certain embodiments, one or more NH groups attached to enzyme in the compound of formula VIII are derived from the primary amino groups of lysine residues. In further embodiments of compound VIII, one or more NH groups attached to enzyme are derived from one or more of the primary amino groups of lysine 199, lysine 211 and lysine 376 (using the numbering of full length human IDS isoform a).

In another embodiment of the invention, the compound includes Angiopep-2 joined to IDS, an active IDS fragment or an active IDS analog via a DBCO containing linker and has the structure

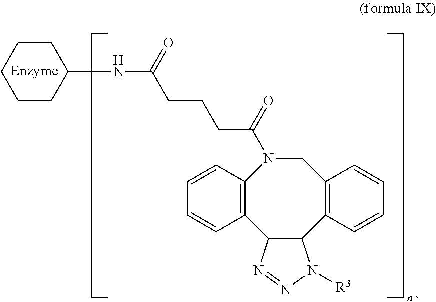

wherein R³ is:

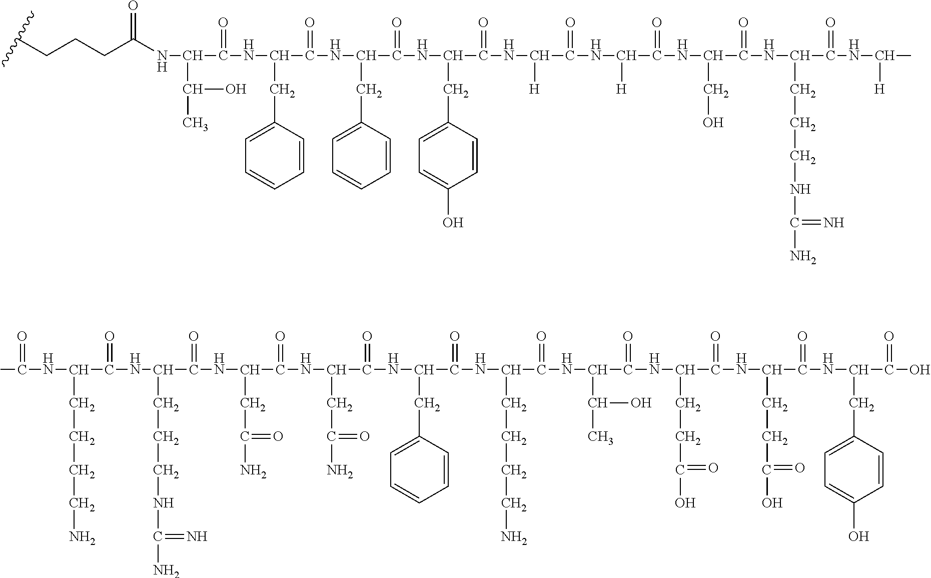

where enzyme represents IDS, an active fragment of IDS or an active analog of IDS, where n is an integer between 1 and 6 and where the NH group attached to the enzyme is derived from the reaction of a primary amino group in the enzyme.

The invention features a population of compounds of formula IX where the average value of n is between 1 and 6 (e.g., 1, 1.3, 1.5, 2, 2.5, 3, 3.5, 4, 4.5, 5, 5.5, 6). More particularly, the invention features a population of compounds of formula IX where the average value of n is about 1.3.

The invention also features a compound where Angiopep-2-Cys is joined to IDS, an active fragment or active IDS analog via a maleimide containing group and has the structure

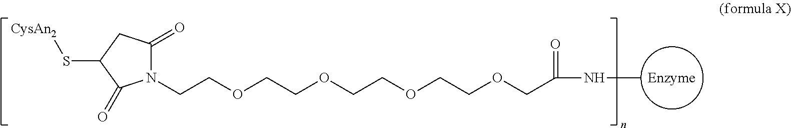

where enzyme represents IDS, an active fragment or active analog of IDS, where n is the number of Angiopep-2 moieties attached to IDS via the linker and is an integer between 1 and 6, wherein the S moiety attached to An₂Cys represents the side chain sulfide on the cysteine in Angiopep-2-Cys, and where the NH group attached to the is derived from the reaction of a primary amino group in the enzyme.

The invention features a population of compounds of formula X where the average value of n is between 0.5 and 6 (e.g., 0.5, 0.8, 1, 1.5, 2, 2.5, 3, 3.5, 4, 4.5, 5, 5.5, or 6). More particularly, the invention features a population of compounds of formula X where the average value of n is about 0.8.

In an alternate embodiment, Cys-Angiopep-2 is joined to IDS, an active fragment or an active IDS analog via a maleimide containing group and has the structure

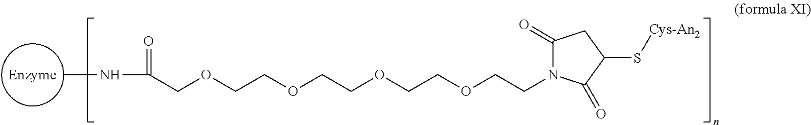

where enzyme represents IDS, an active fragment of IDS or an active analog of IDS, where n is the number of Angiopep-2 moieties attached to IDS via the linker and is between 1 to 6, wherein Cys-An₂ is Cys-Angiopep-2, the S moiety attached to Cys-An₂ represents the side chain sulfide on the cysteine in Cys-Angiopep-2, and where the NH group attached to the enzyme is derived from the reaction of a primary amino group in the enzyme.

The invention features a population of compounds of formula XI where the average value of n is between 0.5 and 6 (e.g., 0.5, 0.9, 1, 1.5, 2, 2.5, 3, 3.5, 4, 4.5, 5, 5.5, or 6). More particularly, the invention features a population of compounds of formula XI where the average value of n is about 0.9.

In one aspect, the linker can be a maleimide containing group functionalized with an alkyne group selected from the group consisting of monofluorocyclooctyne (MFCO), difluorocyclooctyne (DFCO), cyclooctyne (OCT), dibenzocyclooctyne (DIBO), biarylazacyclooctyne (BARAC), difluorobenzocyclooctyne (DIFBO), and bicyclo[6.1.0]nonyne (BCN) and the alkyne-functionalized maleimide is attached to an Angiopep-2 via an azido group attached to Angiopep-2.

In one embodiment of the invention, the compound includes Angiopep-2 joined to IDS via an S-acetylthioacetate (SATA) group and has the structure

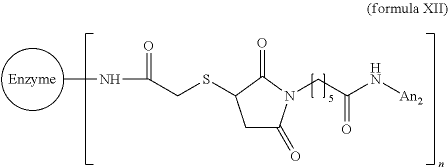

where n is the number of Angiopep-2 moieties attached to IDS via the linker and is between 1-6, An₂ is Angiopep-2, the NH group attached to An2 is the N-terminus amino group of Angiopep-2, and the NH group attached to IDS represents primary amino group in IDS. The invention features a composition comprising the compound of formula XII where the average value of n is between 1 and 6 (e.g., 1, 1.5, 2, 2.5, 2.6, 3, 3.5, 4, 4.5, 5, 5.5, or 6).

The compounds of formulae III to XII described above can have 1, 2, 3, 4, 5, or 6 peptide targeting moieties attached to the enzyme via a linker. In one embodiment, the enzyme is human full length IDS isoform a or human mature IDS isoform a.

In another aspect, the invention features a population of compounds having the general structure:

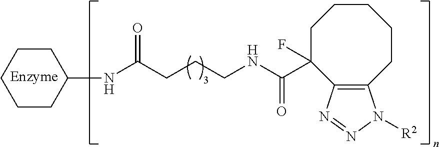

wherein R² is:

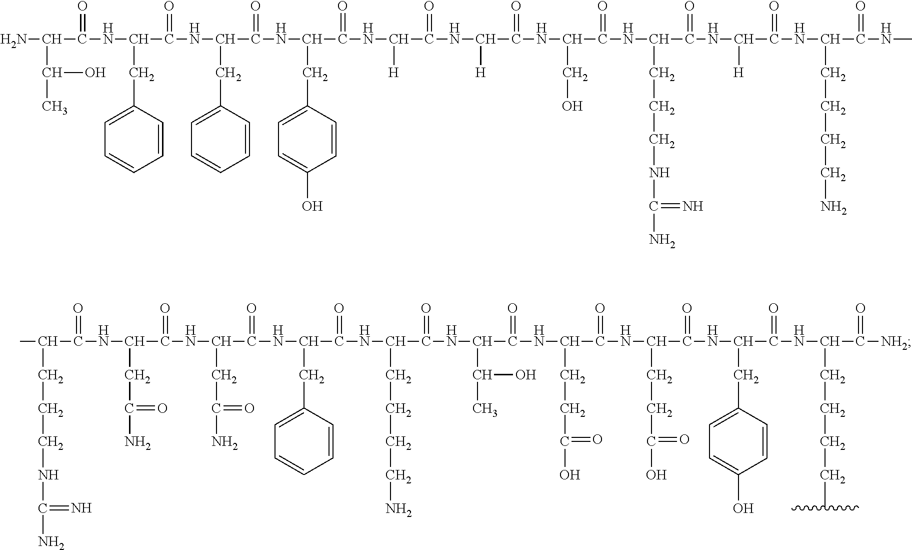

where enzyme represents IDS, an active fragment of IDS or an active analog of IDS, where the NH group attached to the enzyme is derived from the reaction of a primary amino group in the enzyme and where the average value of n is between 1 and 6. In some embodiments the average value of n is between 4.5 and 5.5. In other embodiments the average value of n is about 4.9.

In another aspect, the invention features a population of compounds having the general structure:

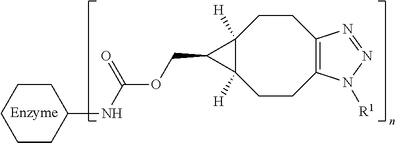

wherein R¹ is:

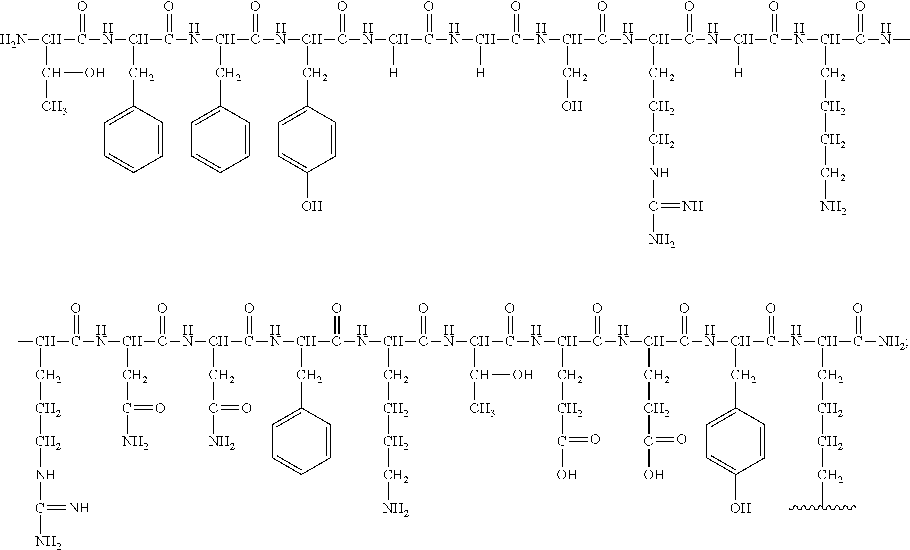

where the enzyme represents IDS, an active fragment of IDS or an active analog of IDS, where the NH group attached to the enzyme is derived from the reaction of a primary amino group in the enzyme and where the average value of n is between 1 and 6. In some embodiments the average value of n is between 2 and 3. In other embodiments the average value of n is about 2.3.

In a further aspect, the invention features compounds that are intermediates in the manufacture of the compounds of the invention having the general formula:

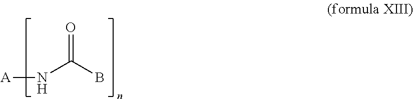

wherein A is an enzyme selected from the group consisting of iduronate-2-sulfatase (IDS), an IDS fragment having IDS activity, or an IDS analog having IDS activity; the NH group attached to A is derived from the reaction of a primary amino group in A; n is an integer between 1 and 8; and B is hydroxyl, optionally substituted C₁₋₁₀ alkyl, optionally substituted C₁₋₁₀alkenyl, optionally substituted alkynyl, optionally substituted aryl, heterocycle, optionally substituted C₁₋₁₀ alkoxy, optionally substituted C₁₋₁₀ alkylamino, optionally substituted C₃₋₁₀ cycloalkyl, optionally substituted C₄₋₁₀ cycloalkenyl, optionally substituted C₄₋₁₀ cycloalkynyl, an amino acid, or a peptide of 2 to 5 amino acids.

In certain embodiments, B is an amino acid, a peptide of 2 to 5 amino acids, or selected from:

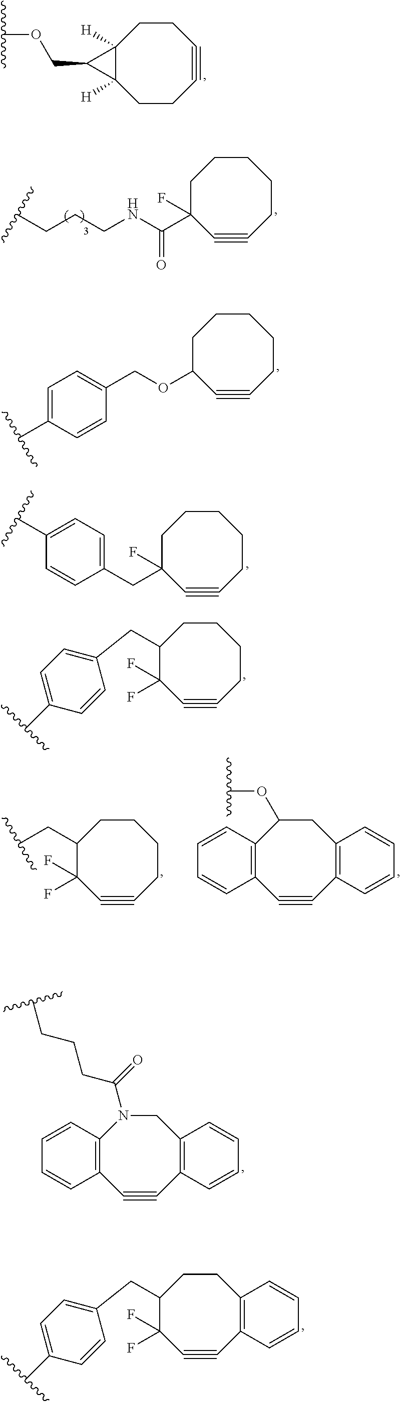

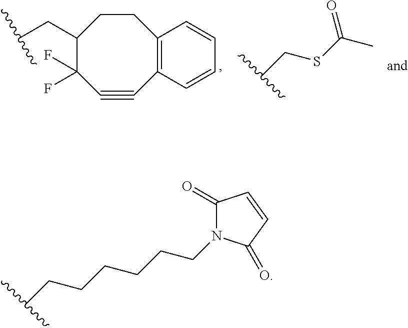

In some embodiments, B is

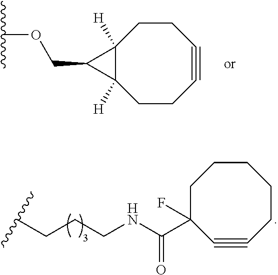

In certain embodiments of these intermediates of the invention, A is modified through derivization of one or more side chain primary amine groups of a lysine.

When B

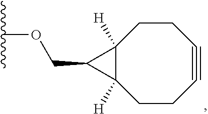 [SMILES: *OC[C@@H]1[C@@H]2CCC#CCC[C@@H]21]

is one or more NH groups attached to A may be derived from the primary amino groups of one or more lysine residues. More particularly, one or more NH groups attached to A are derived from one or more of the primary amino groups of lysine 199, lysine 240, lysine 295, lysine 347, lysine 479 and lysine 483 (using the numbering of full length human IDS isoform a). In one embodiment, one or more NH groups attached to A are derived from one or more of the primary amino groups of lysine 199, lysine 479 and lysine 483 (using the numbering of IDS isoform a).

When B is

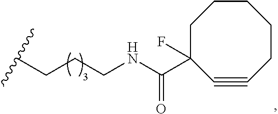

one or more NH groups attached to A may be derived from the primary amino groups of lysine residues. More particularly, an NH group attached to A is derived from lysine 479 (using the numbering of full length human IDS isoform a).

Certain intermediates of the invention exhibit higher levels of IDS activity than the corresponding unmodified IDS, IDS fragment or IDS analog.

The invention features a composition that includes nanoparticles which are conjugated to any of the compounds described above. The invention also features a liposome formulation of any of the compounds featured above.

The invention features a pharmaceutical composition that includes any one of the compounds described above and a pharmaceutically acceptable carrier. The invention also features a method of treating or treating prophylactically a subject having a lysosomal storage disorder, where the method includes administering to a subject any of the above described compounds or compositions. In one aspect of the method, the lysosomal storage disorder is mucopolysaccharidosis Type II (MPS-II) and the lysosomal enzyme is IDS, an active fragment or an active analog thereof. In another aspect of the method, the subject has the severe form of MPS-II or the attenuated form of MPS-II. In yet another aspect of the method, the subject has neurological symptoms. The subject can start treatment at under five years of age, preferably under three years of age. The subject can be an infant. The methods of the invention also include parenteral administration of the compounds and compositions of the invention.

By “subject” is meant a human or non-human animal (e.g., a mammal).

By “lysosomal enzyme” is meant any enzyme that is found in the lysosome in which a defect in that enzyme can lead to a lysosomal storage disorder.

By “lysosomal storage disorder” is meant any disease caused by a defect in a lysosomal enzyme. Approximately fifty such disorders have been identified.

By “targeting moiety” is meant a compound or molecule such as a peptide or a peptidomimetic that can be transported into a particular cell type (e.g., liver, lungs, kidney, spleen, or muscle), into particular cellular compartments (e.g., the lysosome), or across the BBB. In certain embodiments, the targeting moiety may bind to receptors present on brain endothelial cells and thereby be transported across the BBB by transcytosis. The targeting moiety may be a molecule for which high levels of transendothelial transport may be obtained, without affecting the cell or BBB integrity. The targeting moiety may be a peptide or a peptidomimetic and may be naturally occurring or produced by chemical synthesis or recombinant genetic technology.

By “treating” a disease, disorder, or condition in a subject is meant reducing at least one symptom of the disease, disorder, or condition by administrating a therapeutic agent to the subject.

By “treating prophylactically” a disease, disorder, or condition in a subject is meant reducing the frequency of occurrence of or reducing the severity of a disease, disorder or condition by administering a therapeutic agent to the subject prior to the onset of disease symptoms.

By a peptide which is “efficiently transported across the BBB” is meant a peptide that is able to cross the BBB at least as efficiently as Angiopep-6 (i.e., greater than 38.5% that of Angiopep-1 (250 nM) in the in situ brain perfusion assay described in U.S. patent application Ser. No. 11/807,597, filed May 29, 2007, hereby incorporated by reference). Accordingly, a peptide which is “not efficiently transported across the BBB” is transported to the brain at lower levels (e.g., transported less efficiently than Angiopep-6).

By a peptide or compound which is “efficiently transported to a particular cell type” is meant that the peptide or compound is able to accumulate (e.g., either due to increased transport into the cell, decreased efflux from the cell, or a combination thereof) in that cell type to at least a 10% (e.g., 25%, 50%, 100%, 200%, 500%, 1,000%, 5,000%, or 10,000%) greater extent than either a control substance, or, in the case of a conjugate, as compared to the unconjugated agent. Such activities are described in detail in International Application Publication No. WO 2007/009229, hereby incorporated by reference.

By “substantial identity” or “substantially identical” is meant a peptide, polypeptide or polynucleotide sequence that has the same peptide, polypeptide or polynucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For peptides or polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). For nucleic acids, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

Other features and advantages of the invention will be apparent from the following Detailed Description, the drawings, and the claims.

### BRIEF DESCRIPTION OF THE DRAWINGS

FIG. 1 is a schematic diagram showing the IDS constructs that were generated.

FIG. 2 is an image showing a western blot of cell culture media from CHO—S cells transfected with the indicated constructs using an anti-IDS antibody.

FIG. 3 is a schematic diagram showing the fluorescence assay used to detect IDS activity in the examples described below.

FIG. 4 is a graph showing IDS activity in cell culture media from CHO—S cells transfected with the indicated constructs.

FIG. 5A is a graph showing IDS activity over a seven-day period following transfection of CHO—S cells with the indicated constructs.

FIG. 5B is a set of western blot images showing the expression of either IDS-His or IDS-An2-His over a seven-day period in CHO—S cells.

FIG. 6A is a graph showing reduction of ³⁵S-GAG accumulation in MPS-II fibroblasts upon treatment with media from CHO—S cells expressing the indicated construct.

FIG. 6B is a graph showing reduction in GAG accumulation in MPS-II fibroblasts upon treatment with purified IDS-An2-His.

FIGS. 7A-7C are sequences of isoforms of IDS (isoform a, FIG. 7A; isoform b; FIG. 7B; isoform c, FIG. 7C).

FIG. 8 is a set of images showing coomassie blue staining and western blot detection of IDS (JR-032) and IDS-Angiopep-2 conjugates.

FIGS. 9A-9C are a set of graphs showing MALDI-TOF analysis of 70-56-1B, 70-56-2B and 68-32-2 conjugates.

FIG. 10A shows SEC analysis of 68-32-2, 70-56-1B, 70-56-2B, and 70-66-1B.

FIG. 10B shows SP analysis of 68-32-2, 70-56-1B, 70-56-2B, and 70-66-1B.

FIG. 11 is a schematic showing the protocol for measuring intracellular trafficking of Alexa 488 labeled conjugates using confocal microscopy.

FIG. 12 is a set of confocal micrographs showing localization of Alexa-labeled IDS (upper panel) and Alexa-labeled Angiopep-2-IDS (70-56-2B, lower panel) in U87 cells in comparison to lysotracker dye. Colocalization after a 16 hour uptake is shown in fourth panel (merge). Enzymes were incubated at a concentration of 50 nM for 16 hours at 37° C. Magnification is 100×.

FIG. 13 is a set of confocal micrographs showing localization of Alexa-labeled IDS (upper panel) and Alexa-labeled Angiopep-2-IDS (70-56-2B, lower panel) in U87 cells in comparison to lysotracker dye. Lack of colocalization is shown in fourth panel (merge). Enzymes were incubated at a concentration of 100 nM for 1 hour at 37° C. Magnification is 100×.

FIG. 14 is a set of confocal micrographs showing localization of Alexa-labeled IDS (upper panel) and Alexa-labeled Angiopep-2-IDS (70-56-2B, lower panel) in U87 cells in comparison to lysotracker dye. Colocalization is shown in fourth panel (merge) in yellow. Enzymes were incubated at a concentration of 100 nM for 16 hours at 37° C. Magnification is 100×.

FIG. 15 is a confocal micrograph showing localization of Alexa-labeled IDS and Alexa-labeled Angiopep-2-IDS (70-56-1B) in U87 cells in comparison to lysotracker dye. Enzymes were incubated overnight at a concentration of 50 nM at 37° C. Magnification is 100×. The right panel is a zoomed version of the left panel.

FIG. 16 is a set of confocal micrographs showing uptake and localization of Alexa-labeled IDS and Alexa488-labeled An2-IDS conjugates: 68-32-2, 70-66-1B, 70-56-2B, and 68-27-3 in U-87 cells.

FIGS. 17A and 17B are graphs showing uptake of Alexa488-IDS and Alexa488-An2-IDS (70-56-2B) by U87 cells in 1 hour and 16 hours.

FIGS. 18 and 19 are graphs showing that the Angiopep-2-IDS conjugates show increased uptake into U87 cells and that increasing the incorporation ratio of Angiopep-2-IDS conjugates correlates with increased uptake into cells.

FIG. 20A is a graph showing the enzyme activity of IDS-Angiopep-2 conjugates compared to JR-032. Enzyme activity is expressed as % JR-032 control. For conjugates, number of determinations is between 4 and 8, for JR-032, each bar is the average of 15 determinations.

FIG. 20B is a graph showing the enzyme activity of large scale syntheses of the conjugates 70-56-2B, 70-66-1B and 68-32-2 compared to JR-032.

FIG. 21 is a graph showing GAG concentration measured in MPSII patient fibroblasts treated with unconjugated JR-032 or individual conjugates (4 ng/ml). GAG levels are expressed as % of GAG measured in healthy patient fibroblasts.

FIG. 22 is a graph showing that Angiopep-2-IDS conjugates reduce GAG concentration in MPSII fibroblasts with similar potency to unconjugated JR-032. GAG concentration was measured in MPSII patient fibroblasts treated with JR-032 of three conjugates at various concentrations. GAG levels are expressed as % of GAG measured in healthy patient fibroblasts.

FIG. 23 is a graph showing the brain distribution of unconjugated JR-032 and 15 conjugates respectively at a single time point (2 minutes). Unless the C-terminus is specified, all linkers are connected to An2 by N-terminal attachment.

FIGS. 24A and 24B are graphs showing the distribution of JR-032 in different parts of the brain.

FIGS. 24C and 24D are graphs comparing the Kᵢₙ and brain distribution of An2-IDS conjugates with that of unconjugated JR-032.

FIG. 25 is a graph comparing the brain uptake and distribution of JR-032 and inulin.

FIG. 26 is a graph comparing the brain distribution of large scale syntheses of 70-56-2B, 70-66-1B and 68-32-2 in total brain, capillary and parenchyma.

FIG. 27A-27C shows plasma concentration time curves for radiolabelled JR-032 and An2 IDS conjugates.

FIGS. 28A-28C show the concentration of radiolabelled JR-032 and An2-IDS conjugates in tissues at 1 hour, 8 hours and 48 hours.

FIG. 29 shows concentration of radiolabelled JR-032 and An2-IDS conjugates in brain.

FIG. 30 shows concentrations of conjugates compared with JR-032 in brain, heart, liver, lungs, kidney (cortex), muscle (leg abductor), skin, bone (femur including marrow) and spleen at 1 hour post dose and 8 hours post dose.

FIG. 31 shows the concentration of JR-032, 70-66-1B and 68-32-2 at 0.5 hours, 1 hour, 4 hours and 24 hours in plasma, brain, liver and thyroid.

FIGS. 32A and 32B show processing of JR-032, ANG3402, and ANG3403 in liver samples from a PK distribution experiment.

FIG. 33 shows processing of JR-032, ANG3402, and ANG3403 in MPS II fibroblasts.

FIG. 34 shows processing of JR-032, ANG3402, and ANG3403 in plasma.

FIGS. 35A-C are graphs showing GAG concentration in liver, heart and brain of hemizygous knock out mice administered with vehicle, JR-032 or An2-IDS conjugates.

FIG. 36 is a graph showing GAG reduction at each dose for each conjugate (expressed as a percentage of the reduction achieved by JR-032).

### DETAILED DESCRIPTION

The present invention relates to compounds that include a lysosomal enzyme (e.g., IDS) and a targeting moiety (e.g., Angiopep-2) joined by a linker (e.g., a peptide bond). The targeting moiety is capable of transporting the enzyme to the lysosome and/or across the BBB. Such compounds are exemplified by Angiopep-2-IDS conjugates and fusion proteins. These proteins maintain IDS enzymatic activity both in an enzymatic assay and in a cellular model of MPS-II. Because targeting moieties such as Angiopep-2 are capable of transporting proteins across the BBB, these conjugates are expected to have not only peripheral activity, but have activity in the central nervous system (CNS). In addition, targeting moieties such as Angiopep-2 are taken up by cells by receptor mediated transport mechanism (such as LRP-1) into lysosomes. Accordingly, we believe that these targeting moieties can increase enzyme concentrations in the lysosome, thus resulting in more effective therapy, particular in tissues and organs that express the LRP-1 receptor, such as liver, kidney, and spleen.

These features overcome some of the biggest disadvantages of current therapeutic approaches because intravenous administration of IDS by itself does not treat CNS disease symptoms. In contrast to physical methods for bypassing the BBB, such intrathecal or intracranial administration, which are highly invasive and thus generally an unattractive solution to the problem of CNS delivery, the present invention allows for noninvasive brain delivery. In addition, improved transport of the therapeutic to the lysosomes may allow for reduced dosing or reduced frequency of dosing, as compared to standard enzyme replacement therapy.

### Lysosomal Storage Disorders

Lysosomal storage disorders are a group of disorders in which the metabolism of lipids, glycoproteins, or mucopolysaccharides is disrupted based on enzyme dysfunction. This dysfunction leads to cellular buildup of the substance that cannot be properly metabolized. Symptoms vary from disease to disease, but problems in the organ systems (liver, heart, lung, and spleen), bones, as well as neurological problems are present in many of these diseases. Typcially, these diseases are caused by rare genetic defects in the relevant enzymes. Most of these diseases are inherited in autosomal recessive fashion, but some, such as MPS-II, are X-linked recessive diseases.

### Lysosomal Enzymes

The present invention may use any lysosomal enzyme known in the art that is useful for treating a lysosomal storage disorder. The compounds of the present invention are exemplified by iduronate-2-sulfatase (IDS; also known as idursulfase). The compounds may include IDS, a fragment of IDS that retains enzymatic activity, or an IDS analog, which may include amino acid sequences substantially identical (e.g., at least 70, 80, 85, 90, 95, 96, 97, 98, or 99% identical) to the human IDS sequence and retains enzymatic activity.

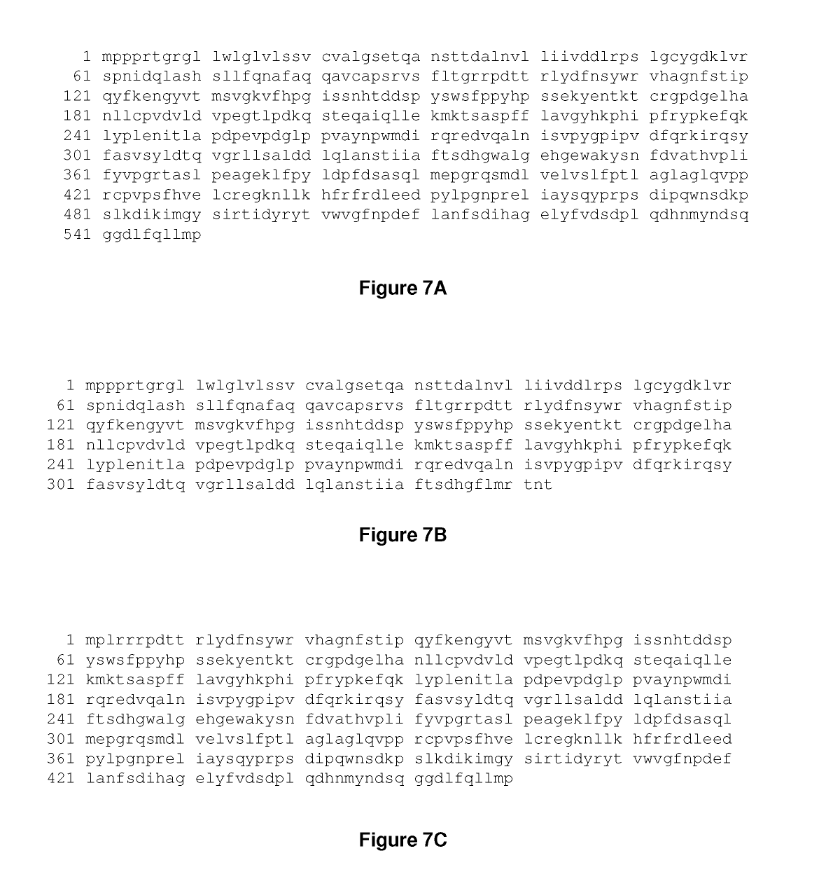

Three isoforms of IDS are known, isoforms a, b, and c. Isoform a is a 550 amino acid protein and is shown in FIG. 7A. Isoform b (FIG. 7B) is a 343 amino acid protein which has a different C-terminal region as compared to the longer Isoform a. Isoform c (FIG. 7C) has changes at the N-terminal due to the use of a downstream start codon. Any of these isoforms may be used in the compounds of the invention.

Recombinant iduronate-2-sulfatase enzymes (e.g., JR-032) are known in the art. JR-032 is a recombinant human IDS full length isoform a (INN: idursulfase) manufactured as described in U.S. Pat. No. 5,932,211.

To test whether particular fragment or analog has enzymatic activity, the skilled artisan can use any appropriate assay. Assays for measuring IDS activity, for example, are known in art, including those described in Hopwood, Carbohydr. Res. 69:203-16, 1979, Bielicki et al., Biochem. J. 271:75-86, 1990, and Dean et al., Clin. Chem. 52:643-9, 2006. A similar fluorometric assay is also described below. Using any of these assays, the skilled artisan would be able to determine whether a particular IDS fragment or analog has enzymatic activity. These assays can also identify whether compounds of the invention have enzyme activity.

In certain embodiments, an enzyme fragment (e.g., an IDS fragment) is used. IDS fragments may be at least 50, 100, 150, 200, 250, 300, 350, 400, 450, or 500 amino acids in length. In certain embodiments, the enzyme may be modified, e.g., using any of the polypeptide modifications described herein.

### Targeting Moieties

The compounds of the invention can feature any of targeting moieties described herein, for example, any of the peptides described in Table 1 (e.g., Angiopep-1, Angiopep-2, or reversed Angiopep-2), or a fragment or peptidomimetic thereof. In certain embodiments, the peptide may have at least 35%, 40%, 50%, 60%, 70%, 80%, 90%, 95%, 99%, or even 100% identity to a peptide described herein. The polypeptide may have one or more (e.g., 2, 3, 4, 5, 6, 7, 8, 9, 10, 11, 12, 13, 14, or 15) substitutions relative to one of the sequences described herein. Other modifications are described in greater detail below.

The invention also features fragments of these peptides or peptidomimetics (e.g., a functional fragment). In certain embodiments, the fragments are capable of efficiently being transported to or accumulating in a particular cell type (e.g., liver, eye, lung, kidney, or spleen) or are efficiently transported across the BBB. Truncations of the peptide or peptidomimetic may be 1, 2, 3, 4, 5, 6, 7, 8, 9, 10, 11, 12, or more amino acids from either the N-terminus of the peptide, the C-terminus of the peptide, or a combination thereof. Other fragments include sequences where internal portions of the peptide or peptidomimetic are deleted.

Additional peptides or peptidomimetics may be identified by using one of the assays or methods described herein. For example, a candidate peptide or peptidomimetic may be produced by conventional peptide synthesis, conjugated with paclitaxel and administered to a laboratory animal. A biologically-active conjugate may be identified, for example, based on its ability to increase survival of an animal injected with tumor cells and treated with the conjugate as compared to a control which has not been treated with a conjugate (e.g., treated with the unconjugated agent). As another example, a biologically active peptide or peptidomimetic may be identified based on its location in the parenchyma in an in situ cerebral perfusion assay.

Assays to determine accumulation in other tissues may be performed as well. Labelled conjugates of a peptide or peptidomimetic can be administered to an animal, and accumulation in different organs can be measured. For example, a peptide or peptidomimetic conjugated to a detectable label (e.g., a near-IR fluorescence spectroscopy label such as Cy5.5) allows live in vivo visualization. Such a peptide or peptidomimetic can be administered to an animal, and the presence of the peptide or peptidomimetic in an organ can be detected, thus allowing determination of the rate and amount of accumulation of the peptide or peptidomimetic in the desired organ. In other embodiments, the peptide or peptidomimetic can be labelled with a radioactive isotope (e.g., ¹²⁵I). The peptide or peptidomimetic is then administered to an animal. After a period of time, the animal is sacrificed and the organs are extracted. The amount of radioisotope in each organ can then be measured using any means known in the art. By comparing the amount of a labeled candidate peptide or peptidomimetic in a particular organ relative to the amount of a labeled control peptide or peptidomimetic, the ability of the candidate peptide or peptidomimetic to access and accumulate in a particular tissue can be ascertained. Appropriate negative controls include any peptide or polypeptide known not to be efficiently transported into a particular cell type (e.g., a peptide related to Angiopep that does not cross the BBB, or any other peptide).

Additional sequences are described in U.S. Pat. No. 5,807,980 (e.g., SEQ ID NO:102 herein), U.S. Pat. No. 5,780,265 (e.g., SEQ ID NO:103), U.S. Pat. No. 5,118,668 (e.g., SEQ ID NO:105). An exemplary nucleotide sequence encoding an aprotinin analog atgagaccag atttctgcct cgagccgccg tacactgggc cctgcaaagc tcgtatcatc cgttacttct acaatgcaaa ggcaggcctg tgtcagacct tcgtatacgg cggctgcaga gctaagcgta acaacttcaa atccgcggaa gactgcatgc gtacttgcgg tggtgcttag; SEQ ID NO:106; Genbank accession No. X04666). Other examples of aprotinin analogs may be found by performing a protein BLAST (Genbank: www.ncbi.nlm.nih.gov/BLAST/) using the synthetic aprotinin sequence (or portion thereof) disclosed in International Application No. PCT/CA2004/000011. Exemplary aprotinin analogs are also found under accession Nos. CAA37967 (GI:58005) and 1405218C (GI:3604747).

### Modified Polypeptides

The fusion proteins, targeting moieties, and lysosomal enzymes or fragments used in the invention may have a modified amino acid sequence and be analogs or peptidomimetics. In certain embodiments, the modification does not destroy significantly a desired biological activity (e.g., ability to cross the BBB or enzymatic activity). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modifications may have or may optimize a characteristic of a compound, fusion protein, targeting moiety, lysosomal enzyme or fragment such as in vivo stability, bioavailability, toxicity, immunological activity, immunological identity, and conjugation properties.

Modifications include those by natural processes, such as posttranslational processing, or by chemical modification techniques known in the art. Modifications may occur anywhere in a (poly)peptide including the (poly)peptide backbone, the amino acid side chains and the amino- or carboxy-terminus. The same type of modification may be present in the same or varying degrees at several sites in a given (poly)peptide, and a (poly)peptide may contain more than one type of modification. (Poly)peptides may be branched as a result of ubiquitination, and they may be cyclic, with or without branching. Cyclic, branched, and branched cyclic (poly)peptides may result from posttranslational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified (poly)peptide can also include an amino acid insertion, deletion, or substitution, either conservative or non-conservative (e.g., D-amino acids, desamino acids) in the polypeptide sequence (e.g., where such changes do not substantially alter the biological activity of the (poly)peptide). In particular, the addition of one or more cysteine residues to the amino or carboxy terminus of any of the (poly)peptides of the invention can facilitate conjugation of these (poly)peptides by, e.g., disulfide bonding. For example, Angiopep-1 (SEQ ID NO:67), Angiopep-2 (SEQ ID NO:97), or Angiopep-7 (SEQ ID NO:112) can be modified to include a single cysteine residue at the amino-terminus (SEQ ID NOS: 71, 113, and 115, respectively) or a single cysteine residue at the carboxy-terminus (SEQ ID NOS: 72, 114, and 116, respectively). Amino acid substitutions can be conservative (i.e., wherein a residue is replaced by another of the same general type or group) or non-conservative (i.e., wherein a residue is replaced by an amino acid of another type). In addition, a non-naturally occurring amino acid can be substituted for a naturally occurring amino acid (i.e., non-naturally occurring conservative amino acid substitution or a non-naturally occurring non-conservative amino acid substitution).

(Poly)peptides made synthetically can include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs or peptidomimetics may be generated by substitutional mutagenesis and retain the biological activity of the original (poly)peptide. Examples of substitutions identified as “conservative substitutions” are shown in Table 2. If such substitutions result in a change not desired, then other type of substitutions, denominated “exemplary substitutions” in Table 2, or as further described herein in reference to amino acid classes, are introduced and the products screened.

Substantial modifications in function or immunological identity are accomplished by selecting substitutions that differ significantly in their effect on maintaining (a) the structure of the (poly)peptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(1) hydrophobic: norleucine, methionine (Met), Alanine (Ala), Valine (Val), Leucine (Leu), Isoleucine (Ile), Histidine (His), Tryptophan (Trp), Tyrosine (Tyr), Phenylalanine (Phe),
(2) neutral hydrophilic: Cysteine (Cys), Serine (Ser), Threonine (Thr)
(3) acidic/negatively charged: Aspartic acid (Asp), Glutamic acid (Glu)
(4) basic: Asparagine (Asn), Glutamine (Gln), Histidine (His), Lysine (Lys), Arginine (Arg)
(5) residues that influence chain orientation: Glycine (Gly), Proline (Pro);
(6) aromatic: Tryptophan (Trp), Tyrosine (Tyr), Phenylalanine (Phe), Histidine (His),
(7) polar: Ser, Thr, Asn, Gln
(8) basic positively charged: Arg, Lys, His, and;
(9) charged: Asp, Glu, Arg, Lys, His

Other amino acid substitutions are listed in Table 2.

TABLE 2
Amino acid substitutions

| | | Conservative |
|---|---|---|
| Original residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Polypeptide Derivatives and Peptidomimetics

In addition to (poly)peptides consisting of naturally occurring amino acids, peptidomimetics or (poly)peptide analogs are also encompassed by the present invention and can form the fusion proteins, targeting moieties, or lysosomal enzymes, enzyme fragments, or enzyme analogs used in the compounds of the invention. (Poly)peptide analogs are commonly used in the pharmaceutical industry as non-peptide drugs with properties analogous to those of the template polypeptide. The non-peptide compounds are termed “peptide mimetics” or peptidomimetics (Fauchere et al., Infect. Immun. 54:283-287, 1986 and Evans et al., J. Med. Chem. 30:1229-1239, 1987). Peptide mimetics that are structurally related to therapeutically useful peptides or polypeptides may be used to produce an equivalent or enhanced therapeutic or prophylactic effect. Generally, peptidomimetics are structurally similar to the paradigm (poly)peptide (i.e., a (poly)peptide that has a biological or pharmacological activity) such as naturally-occurring receptor-binding (poly)peptides, but have one or more peptide linkages optionally replaced by linkages such as —CH₂NH—, —CH₂S—, —CH₂—CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such peptidomimetics may have significant advantages over naturally occurring (poly)peptides including more economical production, greater chemical stability, enhanced pharmacological properties (e.g., half-life, absorption, potency, efficiency), reduced antigenicity, and others.

While the peptide targeting moieties described herein may efficiently cross the BBB or target particular cell types (e.g., those described herein), their effectiveness may be reduced by the presence of proteases. Likewise, the effectiveness of the lysosomal enzymes or enzyme fragments used in the compounds of the invention may be similarly reduced. Serum proteases have specific substrate requirements, including L-amino acids and peptide bonds for cleavage. Furthermore, exopeptidases, which represent the most prominent component of the protease activity in serum, usually act on the first peptide bond of the polypeptide and require a free N-terminus (Powell et al., Pharm. Res. 10:1268-1273, 1993). In light of this, it is often advantageous to use modified versions of the targeting peptidesm enzymes or enzyme fragments. The peptidomimetics or analogs retain the structural characteristics of the original L-amino acid (poly)peptides, but advantageously are not readily susceptible to cleavage by protease and/or exopeptidases.

Systematic substitution of one or more amino acids of a consensus sequence with D-amino acid of the same type (e.g., an enantiomer; D-lysine in place of L-lysine) may be used to generate more stable (poly)peptides. Thus, a (poly)peptide derivative, analog or peptidomimetic as described herein may be all L-, all D-, or mixed D, L polypeptides. The presence of an N-terminal or C-terminal D-amino acid increases the in vivo stability of a (poly)peptide because peptidases cannot utilize a D-amino acid as a substrate (Powell et al., Pharm. Res. 10:1268-1273, 1993). Reverse-D (poly)peptides are (poly)peptides containing D-amino acids, arranged in a reverse sequence relative to a (poly)peptide containing L-amino acids. Thus, the C-terminal residue of an L-amino acid (poly)peptide becomes N-terminal for the D-amino acid (poly)peptide, and so forth. Reverse D-(poly)peptides may retain the same tertiary conformation and therefore the same activity, as the L-amino acid polypeptides, but are more stable to enzymatic degradation in vitro and in vivo, and thus have greater therapeutic efficacy than the original (poly)peptide (Brady and Dodson, Nature 368:692-693, 1994 and Jameson et al., Nature 368:744-746, 1994). In addition to reverse-D-(poly)peptides, constrained (poly)peptides comprising a consensus sequence or a substantially identical consensus sequence variation may be generated by methods well known in the art (Rizo et al., Ann. Rev. Biochem. 61:387-418, 1992). For example, constrained (poly)peptides may be generated by adding cysteine residues capable of forming disulfide bridges and, thereby, resulting in a cyclic (poly)peptide. Cyclic (poly)peptides have no free N- or C-termini. Accordingly, they are not susceptible to proteolysis by exopeptidases, although they are, of course, susceptible to endopeptidases, which do not cleave at (poly)peptide termini. The amino acid sequences of the (poly)peptides with N-terminal or C-terminal D-amino acids and of the cyclic (poly)peptides are usually identical to the sequences of the (poly)peptides to which they correspond, except for the presence of N-terminal or C-terminal D-amino acid residue, or their circular structure, respectively.

A cyclic derivative containing an intramolecular disulfide bond may be prepared by conventional solid phase synthesis while incorporating suitable S-protected cysteine or homocysteine residues at the positions selected for cyclization such as the amino and carboxy termini (Sah et al., J. Pharm. Pharmacol. 48:197, 1996). Following completion of the chain assembly, cyclization can be performed either (1) by selective removal of the S-protecting group with a consequent on-support oxidation of the corresponding two free SH-functions, to form a S—S bonds, followed by conventional removal of the product from the support and appropriate purification procedure or (2) by removal of the polypeptide from the support along with complete side chain de-protection, followed by oxidation of the free SH-functions in highly dilute aqueous solution.

The cyclic derivative containing an intramolecular amide bond may be prepared by conventional solid phase synthesis while incorporating suitable amino and carboxyl side chain protected amino acid derivatives, at the position selected for cyclization. The cyclic derivatives containing intramolecular —S-alkyl bonds can be prepared by conventional solid phase chemistry while incorporating an amino acid residue with a suitable amino-protected side chain, and a suitable S-protected cysteine or homocysteine residue at the position selected for cyclization.

Another effective approach to confer resistance to peptidases acting on the N-terminal or C-terminal residues of a (poly)peptide is to add chemical groups at the polypeptide termini, such that the modified (poly)peptide is no longer a substrate for the peptidase. One such chemical modification is glycosylation of the (poly)peptides at either or both termini. Certain chemical modifications, in particular N-terminal glycosylation, have been shown to increase the stability of (poly)peptides in human serum (Powell et al., Pharm. Res. 10:1268-1273, 1993). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified (poly)peptides consisting of polypeptides bearing an N-terminal acetyl group and/or a C-terminal amide group.

Also included by the present invention are other types of (poly)peptide derivatives, analogs or peptidomimetics containing additional chemical moieties not normally part of the (poly)peptide, provided that the derivative, analog or peptidomimetic retains the desired functional activity of the (poly)peptide. Examples of such derivatives, analogs or peptidomimetics include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives.

Longer (poly)peptide sequences which result from the addition of additional amino acid residues to the polypeptides described herein are also encompassed in the present invention. Such longer polypeptide sequences can be expected to have the same biological activity and specificity (e.g., cell tropism) as the polypeptides described above. While polypeptides having a substantial number of additional amino acids are not excluded, it is recognized that some large polypeptides may assume a configuration that masks the effective sequence, thereby preventing binding to a target (e.g., a member of the LRP receptor family). These derivatives could act as competitive antagonists. Thus, while the present invention encompasses polypeptides or derivatives of the polypeptides described herein having an extension, desirably the extension does not destroy the cell targeting activity or enzymatic activity of the compound.

Other derivatives included in the present invention are dual polypeptides consisting of two of the same, or two different polypeptides, as described herein, covalently linked to one another either directly or through a spacer, such as by a short stretch of alanine residues or by a putative site for proteolysis (e.g., by cathepsin, see e.g., U.S. Pat. No. 5,126,249 and European Patent No. 495 049). Multimers of the polypeptides described herein consist of a polymer of molecules formed from the same or different polypeptides or derivatives thereof.

The present invention also encompasses polypeptide derivatives that are chimeric or fusion proteins containing a polypeptide described herein, or fragment thereof, linked at its amino- or carboxy-terminal end, or both, to an amino acid sequence of a different protein. Such a chimeric or fusion protein may be produced by recombinant expression of a nucleic acid encoding the protein. For example, a chimeric or fusion protein may contain at least 6 amino acids shared with one of the described polypeptides which desirably results in a chimeric or fusion protein that has an equivalent or greater functional activity.

Assays to Identify Peptidomimetics

As described above, non-peptidyl compounds generated to replicate the backbone geometry and pharmacophore display (peptidomimetics) of the polypeptides described herein often possess attributes of greater metabolic stability, higher potency, longer duration of action, and better bioavailability.

Peptidomimetics compounds can be obtained using any of the numerous approaches in combinatorial library methods known in the art, including biological libraries, spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer, or small molecule libraries of compounds (Lam, Anticancer Drug Des. 12:145, 1997). Examples of methods for the synthesis of molecular libraries can be found in the art, for example, in: DeWitt et al. (Proc. Natl. Acad. Sci. USA 90:6909, 1993); Erb et al. (Proc. Natl. Acad. Sci. USA 91:11422, 1994); Zuckermann et al. (J. Med. Chem. 37:2678, 1994); Cho et al. (Science 261:1303, 1993); Carell et al. (Angew. Chem, Int. Ed. Engl. 33:2059, 1994 and ibid 2061); and in Gallop et al. (Med. Chem. 37:1233, 1994). Libraries of compounds may be presented in solution (e.g., Houghten, Biotechniques 13:412-421, 1992) or on beads (Lam, Nature 354:82-84, 1991), chips (Fodor, Nature 364:555-556, 1993), bacteria or spores (U.S. Pat. No. 5,223,409), plasmids (Cull et al., Proc. Natl. Acad. Sci. USA 89:1865-1869, 1992) or on phage (Scott and Smith, Science 249:386-390, 1990), or luciferase, and the enzymatic label detected by determination of conversion of an appropriate substrate to product.

Once a polypeptide as described herein is identified, it can be isolated and purified by any number of standard methods including, but not limited to, differential solubility (e.g., precipitation), centrifugation, chromatography (e.g., affinity, ion exchange, and size exclusion), or by any other standard techniques used for the purification of peptides, peptidomimetics, or proteins. The functional properties of an identified polypeptide of interest may be evaluated using any functional assay known in the art. Desirably, assays for evaluating downstream receptor function in intracellular signaling are used (e.g., cell proliferation).

For example, the peptidomimetics compounds of the present invention may be obtained using the following three-phase process: (1) scanning the polypeptides described herein to identify regions of secondary structure necessary for targeting the particular cell types described herein; (2) using conformationally constrained dipeptide surrogates to refine the backbone geometry and provide organic platforms corresponding to these surrogates; and (3) using the best organic platforms to display organic pharmocophores in libraries of candidates designed to mimic the desired activity of the native polypeptide. In more detail the three phases are as follows. In phase 1, the lead candidate polypeptides are scanned and their structure abridged to identify the requirements for their activity. A series of polypeptide analogs of the original are synthesized. In phase 2, the best polypeptide analogs are investigated using the conformationally constrained dipeptide surrogates. Indolizidin-2-one, indolizidin-9-one and quinolizidinone amino acids (I²aa, I⁹aa and Qaa respectively) are used as platforms for studying backbone geometry of the best peptide candidates. These and related platforms (reviewed in Halab et al., Biopolymers 55:101-122, 2000 and Hanessian et al., Tetrahedron 53:12789-12854, 1997) may be introduced at specific regions of the polypeptide to orient the pharmacophores in different directions. Biological evaluation of these analogs identifies improved lead polypeptides that mimic the geometric requirements for activity. In phase 3, the platforms from the most active lead polypeptides are used to display organic surrogates of the pharmacophores responsible for activity of the native peptide. The pharmacophores and scaffolds are combined in a parallel synthesis format. Derivation of polypeptides and the above phases can be accomplished by other means using methods known in the art.

Structure function relationships determined from the polypeptides, polypeptide derivatives, peptidomimetics or other small molecules described herein may be used to refine and prepare analogous molecular structures having similar or better properties. Accordingly, the compounds of the present invention also include molecules that share the structure, polarity, charge characteristics and side chain properties of the polypeptides described herein.

In summary, based on the disclosure herein, those skilled in the art can develop peptides and peptidomimetics screening assays which are useful for identifying compounds for targeting an agent to particular cell types (e.g., those described herein). The assays of this invention may be developed for low-throughput, high-throughput, or ultra-high throughput screening formats. Assays of the present invention include assays amenable to automation.

### Linkers

The lysosomal enzyme (e.g., IDS), enzyme fragment, or enzyme analog may be bound to the targeting moiety either directly (e.g., through a covalent bond such as a peptide bond) or may be bound through a linker. Linkers include chemical linking agents (e.g., cleavable linkers) and peptides.

In some embodiments, the linker is a chemical linking agent. The lysosomal enzyme (e.g., IDS), enzyme fragment, or enzyme analog and targeting moiety may be conjugated through sulfhydryl groups, amino groups (amines), and/or carbohydrates or any appropriate reactive group. Homobifunctional and heterobifunctional cross-linkers (conjugation agents) are available from many commercial sources. Regions available for cross-linking may be found on the polypeptides of the present invention. The cross-linker may comprise a flexible arm, e.g., 2, 3, 4, 5, 6, 7, 8, 9, 10, 11, 12, 13, 14, or 15 carbon atoms. Exemplary cross-linkers include BS3 ([Bis(sulfosuccinimidyl)suberate]; BS3 is a homobifunctional N-hydroxysuccinimide ester that targets accessible primary amines), NHS/EDC (N-hydroxysuccinimide and N-ethyl-′(dimethylaminopropyl)carbodimide; NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-S-acetylthioacetate; SATA is reactive towards amines and adds protected sulfhydryls groups).

To form covalent bonds, one can use as a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA).

Primary amines are the principal targets for NHS esters. Accessible a-amine groups present on the N-termini of proteins and the c-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the invention, the functional group on the protein will be a thiol group and the chemically reactive group will be a maleimido-containing group such as gamma-maleimide-butrylamide (GMBA or MPA). Such maleimide containing groups are referred to herein as maleido groups.

The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl can be formed.

In other embodiments, the linker includes at least one amino acid (e.g., a peptide of at least 2, 3, 4, 5, 6, 7, 10, 15, 20, 25, 40, or 50 amino acids). In certain embodiments, the linker is a single amino acid (e.g., any naturally occurring amino acid such as Cys). In other embodiments, a glycine-rich peptide such as a peptide having the sequence [Gly-Gly-Gly-Gly-Ser]ₙ where n is 1, 2, 3, 4, 5 or 6 is used, as described in U.S. Pat. No. 7,271,149. In other embodiments, a serine-rich peptide linker is used, as described in U.S. Pat. No. 5,525,491. Serine rich peptide linkers include those of the formula [X-X-X-X-Gly]_{y}, where up to two of the X are Thr, and the remaining X are Ser, and y is 1 to 5 (e.g., Ser-Ser-Ser-Ser-Gly, where y is greater than 1). In some cases, the linker is a single amino acid (e.g., any amino acid, such as Gly or Cys). Other linkers include rigid linkers (e.g., PAPAP and (PT)ₙP, where n is 2, 3, 4, 5, 6, or 7) and a-helical linkers (e.g., A(EAAAK)ₙA, where n is 1, 2, 3, 4, or 5).

Examples of suitable linkers are succinic acid, Lys, Glu, and Asp, or a dipeptide such as Gly-Lys. When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the c-amino group of Lys and the substituent. In one particular embodiment, the further linker is succinic acid which, e.g., forms an amide bond with the c-amino group of Lys and with an amino group present in the substituent. In one embodiment, the further linker is Glu or Asp (e.g., which forms an amide bond with the c-amino group of Lys and another amide bond with a carboxyl group present in the substituent), that is, the substituent is an N^{ε}-acylated lysine residue.

### Click-Chemistry Linkers

In particular embodiments, the linker is formed by the reaction between a click-chemistry reaction pair. By click-chemistry reaction pair is meant a pair of reactive groups that participates in a modular reaction with high yield and a high thermodynamic gain, thus producing a click-chemistry linker. In this embodiment, one of the reactive groups is attached to the enzyme moiety and the other reactive group is attached to the targeting polypeptide. Exemplary reactions and click-chemistry pairs include a Huisgen 1,3-dipolar cycloaddition reaction between an alkynyl group and an azido group to form a triazole-containing linker; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; a splint ligation reaction with a phosphorothioate group and an iodo group; and a reductive amination reaction with an aldehyde group and an amino group (Kolb et al., Angew. Chem. Int. Ed., 40:2004-2021 (2001); Van der Eycken et al., QSAR Comb. Sci., 26:1115-1326 (2007)).

In particular embodiments of the invention, the polypeptide is linked to the enzyme moiety by means of a triazole-containing linker formed by the reaction between a alkynyl group and an azido group click-chemistry pair. In such cases, the azido group may be attached to the polypeptide and the alkynyl group may be attached to the enzyme moiety. Alternatively, the azido group may be attached to the enzyme moiety and the alkynyl group may be attached to the polypeptide. In certain embodiments, the reaction between an azido group and the alkynyl group is uncatalyzed, and in other embodiments the reaction is catalyzed by a copper(I) catalyst (e.g., copper(I) iodide), a copper(II) catalyst in the presence of a reducing agent (e.g., copper(II) sulfate or copper(II) acetate with sodium ascorbate), or a ruthenium-containing catalyst (e.g., Cp*RuCl(PPh₃)₂ or Cp*RuCl(COD)).

Exemplary linkers include linkers containing monofluorocyclooctyne (MFCO), difluorocyclooctyne (DFCO), cyclooctyne (OCT), dibenzocyclooctyne (DIBO), biarylazacyclooctyne (BARAC), difluorobenzocyclooctyne (DIFBO), and bicyclo[6.1.0]nonyne (BCN).

### Treatment of Lysosomal Storage Disorders

The present invention also features methods for treatment of lysosomal storage disorders such as MPS-II. MPS-II is characterized by cellular accumulation of glycosaminoglycans (GAG) which results from the inability of the individual to break down these products.

In certain embodiments, treatment is performed on a subject who has been diagnosed with a mutation in the IDS gene, but does not yet have disease symptoms (e.g., an infant or subject under the age of 2). In other embodiments, treatment is performed on an individual who has at least one MPS-II symptom (e.g., any of those described herein).

MPS-II is generally classified into two general groups, severe disease and attenuated disease. The present invention can involve treatment of subjects with either type of disease. Severe disease is characterized by CNS involvement. In severe disease the cognitive decline, coupled with airway and cardiac disease, usually results in death before adulthood. The attenuated form of the disease general involves only minimal or no CNS involvement. In both severe and attenuated disease, the non-CNS symptoms can be as severe as those with the “severe” form.

Initial MPS-II symptoms begin to manifest themselves from about 18 months to about four years of age and include abdominal hernias, ear infections, runny noses, and colds. Symptoms include coarseness of facial features (e.g., prominent forehead, nose with a flattened bridge, and an enlarged tongue), large head (macrocephaly), enlarged abdomen, including enlarged liver (heptaomegaly) and enlarged spleen (slenomegaly), and hearing loss. The methods of the invention may involve treatment of subjects having any of the symptoms described herein. MPS-II also results in joint abnormalities, related to thickening of bones.

Treatment may be performed in a subject of any age, starting from infancy to adulthood. Subjects may begin treatment at birth, six months, or 1, 2, 3, 4, 5, 6, 7, 8, 9, 10, 11, 12, 13, 15, or 18 years of age.

### Administration and Dosage

The present invention also features pharmaceutical compositions that contain a therapeutically effective amount of a compound of the invention. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that include the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a subject diagnosed as having mutation associated with a lysosomal storage disorder (e.g., a mutation in the IDS gene). Compositions of the invention can be administered to the subject (e.g., a human) in an amount sufficient to delay, reduce, or preferably prevent the onset of the disorder. In therapeutic applications, compositions are administered to a subject (e.g., a human) already suffering from a lysosomal storage disorder (e.g., MPS-II) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of a lysosomal storage disease, an agent or compound that decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Amounts effective for this use may depend on the severity of the disease or condition and the weight and general state of the subject. Idursulfase is recommended for weekly intravenous administration of 0.5 mg/kg. A compound of the invention may, for example, be administered at an equivalent dosage (i.e., accounting for the additional molecular weight of the fusion protein vs. idursulfase) and frequency. The compound may be administered at an iduronase equivalent dose, e.g., 0.01, 0.05, 0.1, 0.5, 0.1, 0.2, 0.3, 0.4, 0.5, 0.75, 1.0, 1.25, 1.5, 2.0, 2.5, 3.0, 4.0, or 5 mg/kg weekly, twice weekly, every other day, daily, or twice daily. The therapeutically effective amount of the compositions of the invention and used in the methods of this invention applied to mammals (e.g., humans) can be determined by the ordinarily-skilled artisan with consideration of individual differences in age, weight, and the condition of the mammal. Because certain compounds of the invention exhibit an enhanced ability to cross the BBB and to enter lysosomes, the dosage of the compounds of the invention can be lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose of required for a therapeutic effect of the unconjugated agent. The agents of the invention are administered to a subject (e.g. a mammal, such as a human) in an effective amount, which is an amount that produces a desirable result in a treated subject (e.g., reduction of GAG accumulation). Therapeutically effective amounts can also be determined empirically by those of skill in the art.

Single or multiple administrations of the compositions of the invention including an effective amount can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

The compounds of the present invention may be used in combination with either conventional methods of treatment or therapy or may be used separately from conventional methods of treatment or therapy.

When the compounds of this invention are administered in combination therapies with other agents, they may be administered sequentially or concurrently to an individual. Alternatively, pharmaceutical compositions according to the present invention may be comprised of a combination of a compound of the present invention in association with a pharmaceutically acceptable excipient, as described herein, and another therapeutic or prophylactic agent known in the art.

The following examples are intended to illustrate, rather than limit, the invention.

### Example 1

### Design of IDS-Angiopep-2 Fusion Proteins

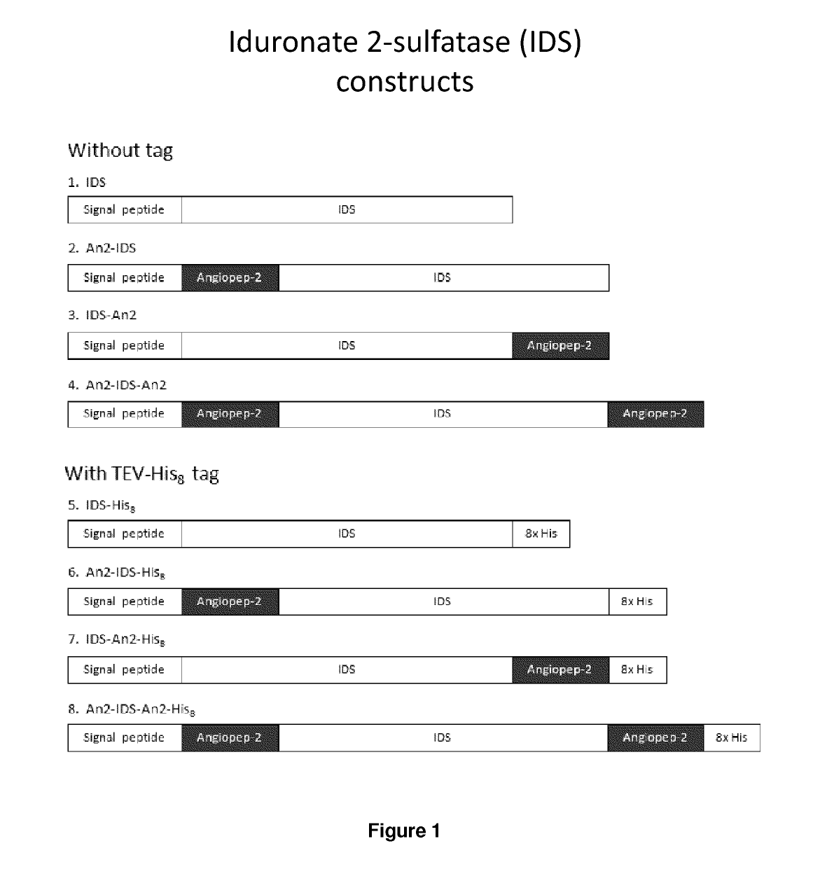

A series of IDS-Angiopep-2 constructs were designed. The IDS cDNA was obtained from Origene (Cat. No. RC219187). Three basic configurations were used: an N-terminal fusion (An2-IDS and An2-IDS-His), a C-terminal fusion (IDS-An2 and IDS-An2-His), and an N- and C-terminal fusion (An2-IDS-An2 and An2-IDS-An2-His), both with and without an 8×His tag (FIG. 1). A control without Angiopep-2 was also generated (IDS and IDS-His).

### Example 2

### Expression and Activity of Recombinant hIDS Proteins in CHO—S Cells

These constructs were then expressed in CHO—S cells grown in suspension. IDS constructs were expressed by transient transfection in FreeStyle CHO—S cells (Invitrogen), using linear 25 kDa polyethyleneimine (PEI, Polyscience) as the transfection reagent. In one example, DNA (1 mg) was mixed with 70 ml FreeStyle CHO Expression medium (Invitrogen) and incubated at room temperature for 15 min PEI (2 mg) was separately incubated in 70 ml medium for 15 minutes, and then DNA and PEI solutions were mixed and further incubated for 15 min. The DNA/PEI complex mixture was added to 360 ml of medium containing 1×10⁹ CHO—S cells. After a four-hour incubation at 37° C., 8% CO₂ with moderate agitation, 500 ml of warm medium was added. CHO—S cells were further incubated for 5 days in the same conditions before harvesting.

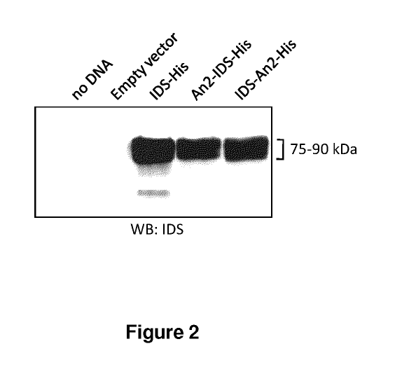

To determine if the cells were expressing and secreting IDS or an IDS fusion protein, a western blot using an anti-IDS antibody was performed on the culture medium. As can be seen in FIG. 2, expression levels of IDS-His, An2-IDS-His and IDS-An2-His were similar. Thus, the cells were able to express these proteins.

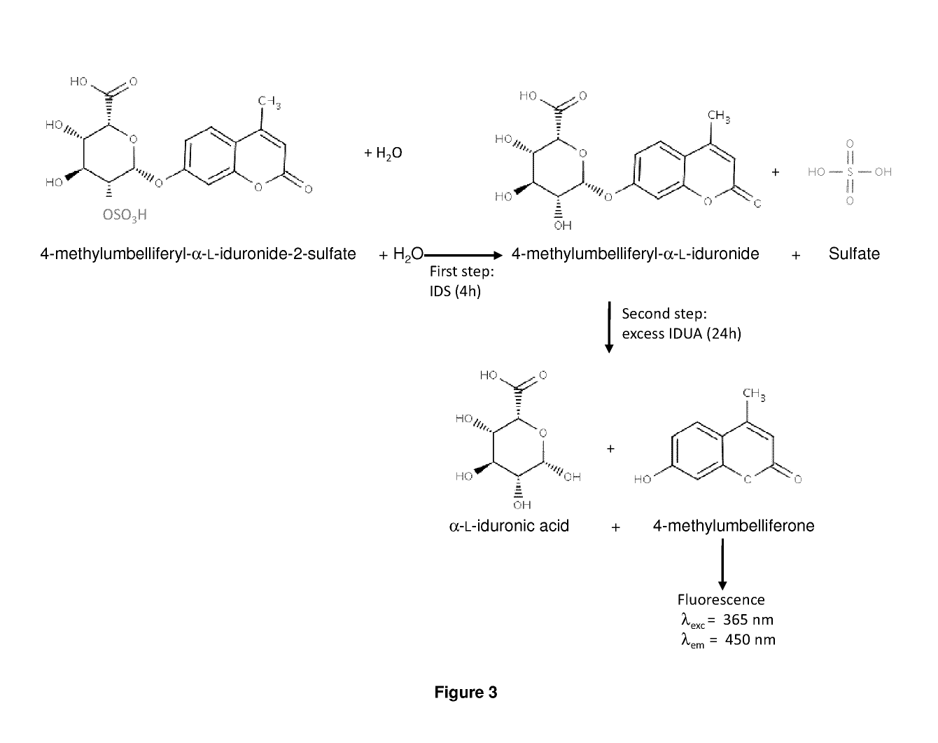

We also characterized IDS activity in the media. This assay was performed using a two-step enzymatic assay (FIG. 3). This assay involves treating 4-methylumbelliferyl-α-L-iduronide-2-sulfate in water with IDS for 4 hours to generate 4-methylumbelliferyl-α-L-iduronide and sulfate. In a second step, these products were treated with excess a-L-iduronidase (IDUA) for 24 hours to generate a-L-iduronic acid and 4-methylumbelliferone. Activity was determined by measuring fluorescence of 4-methylumbelliferone (365 nm excitation; 450 nm emission).

In one particular example, this assay was performed as follows. Ten μl of media from CHO—S transfected cells was mixed with 20 μl of 1.25 mM 4-methylumbelliferyl-alpha-L-iduronide-2-sulphate (IDS substrate from Moscerdam Substrates) in acetate buffer, pH 5.0, and incubated for 4 h at 37° C. The second step of the assay was then initiated by adding 20 μl 0.2 M Na₂HPO₄/0.1 M citric acid buffer, pH 4.5 and 10 μl lysosomal enzymes purified from bovine testis (LEBT). After 24 h at 37° C., the reaction was stopped with 200 μl 0.5 M NaHCO3/Na₂CO3 buffer, pH 10.7, containing 0.025% Triton X-100. Activity was determined by measuring fluorescence of 4-methylumbelliferone (365 nm excitation; 450 nm emission).

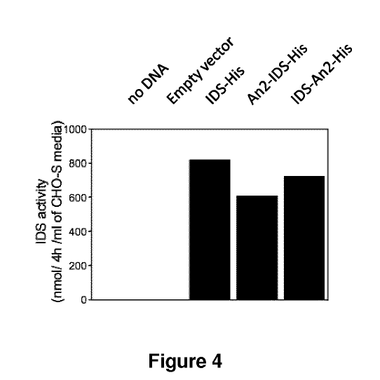

Measurements of IDS activity in the CHO—S cells grown in suspension is shown in FIG. 4, and all three proteins (IDS-His, An2-IDS-His, and IDS-AN2-His) were shown to have IDS activity.

### Example 3

### Characterization and Optimization of Expression

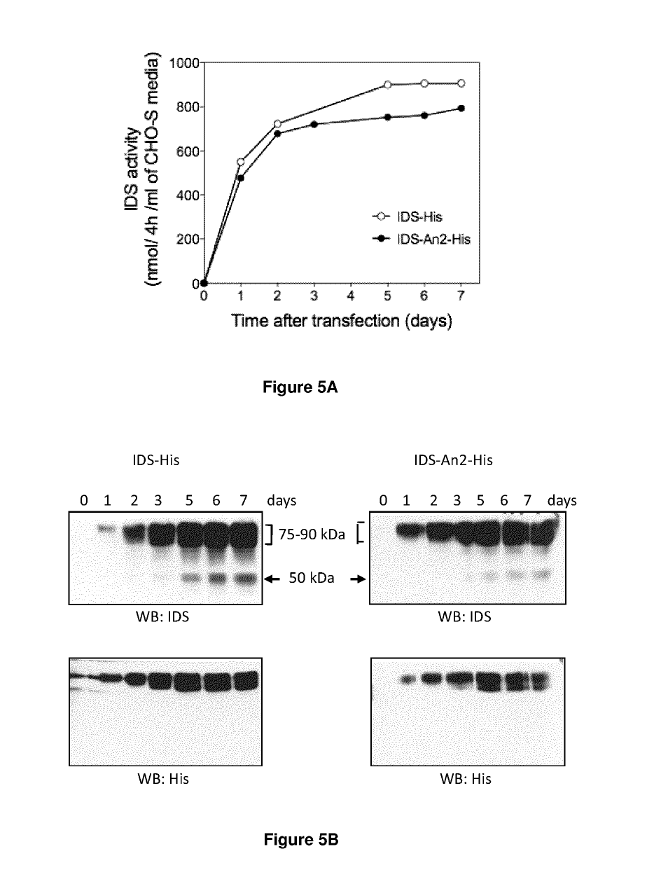

To further characterize expression, time course evaluation of IDS expression and activity in CHO—S cells grown in suspension was measured for the IDS-His and IDS-An2-His fusion proteins as shown in FIG. 5A and FIG. 5B. From these data, maximal IDS expression and activity was observed five days after transfection. No recapture of IDS-An2-His by CHO—S cells was observed in these experiments.

To further optimize transfection conditions, transfection was performed using two different numbers of cells (1.25×10⁷ cells or 2.5×10⁷ cells). Three different ratios of DNA to polyethylenimine (PEI) were used (1:1, 1:2, 1:3, and 1:4).

From these experiments, the best results were obtained using a 1:2 DNA:PEI ratio, as shown by the IDS activity (FIG. 5A) and by expression analysis (FIG. 5B).

### Example 4

### IDS Activity in MPS-II Fibroblasts

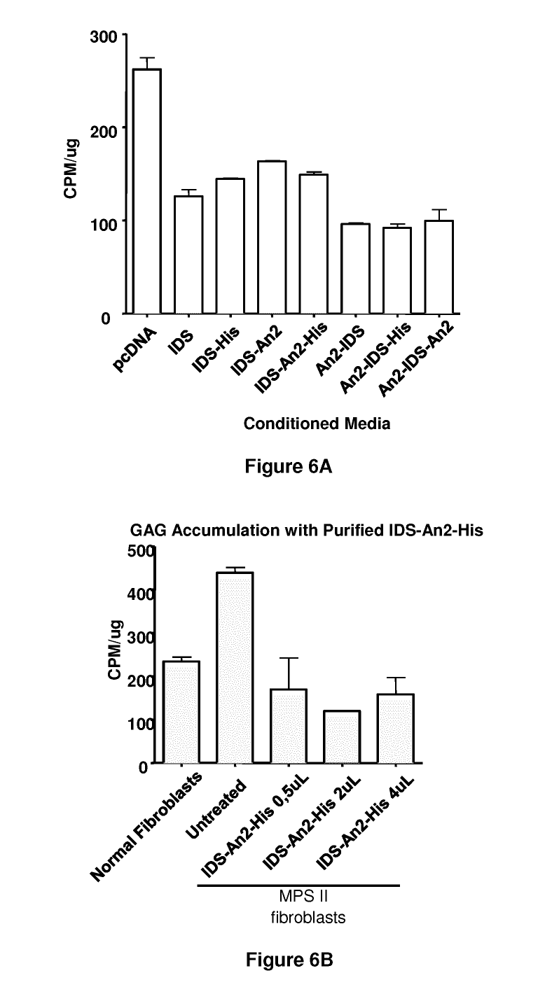

To determine whether, the expressed proteins are capable of reducing glycosaminoglycans (GAG) accumulation in cells, fibroblasts taken from an MPS-II patient were used. In a first set of experiments, cell culture medium from the above-described CHO—S cells transfected with various IDS and IDS fusion proteins was incubated with the fibroblasts. GAG accumulation was measured based on the presence of 35S-GAG. As shown in FIG. 6A, reduction of GAG using the fusion proteins was similar to that of IDS itself.

These assays were performed as follows. MPS II (Coriell institute, GM00298), or healthy human fibroblasts (GM05659) were plated in 6-well dishes at 250,000 cells/well in DMEM with 10% fetal bovine serum (FBS) and grown at 37° C. under 5% CO₂. After 4 days, cells were washed once with PBS and once with low sulfate F-12 medium (Invitrogen, catalog #11765-054). One ml of low sulfate F-12 medium containing 10% dialyzed FBS (Sigma, catalog # F0392) and 10 μCi ³⁵S-sodium sulfate was added to the cells in the absence or presence of recombinant IDS proteins. Fibroblasts were incubated at 37° C. under 5% CO₂. After 48 h, medium was removed and cells were washed 5 times with PBS. Cells were lysed in 0.4 ml/well of 1 N NaOH and heated at 60° C. for 60 min to solubilize proteins. An aliquot was removed for micro-BCA (bicinchoninic acid) protein assay (Smith, P. K. et al., 1986, Anal. Biochem., 150(1): 76-85). Radioactivity was counted with a liquid scintillation counter. The data are expressed as ³⁵S CPM per μg protein.

Even more promising results were obtained with purified IDS-An2-his which was able to decrease the GAG-accumulation to normal control value measured in normal human fibroblasts (FIG. 6B). These results indicate that our purified fusion protein is active. In sum, these data with MPS-II fibroblasts indicate that the fusion proteins are active and that they reach the lysosomes where they can cleave the glycoaminoglycans.

Finally, western blots show that low density lipoprotein receptor-related protein 1 (LRP-1) is expressed at the same levels in normal and MPS-II fibroblasts (data not shown).

### Example 5

### Click Chemistry Linkers

In one example, the targeting moiety is joined to the lysosomal enzyme through a click chemistry linker. An example of this chemistry is shown below.

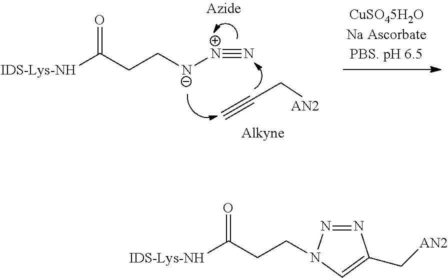

This approach is advantageous in that it is very selective because the reaction only occurs between the azide and alkyne components. The reaction also takes place in aqueous solution and is biocompatible and can be performed in living cells. In addition, the reaction is rapid and quantitative, allowing preparation of nanomoles of conjugates in dilute solutions. Finally, because the reaction is pH-insensitive, it can be performed anywhere from pH 4 to 11. Specific click chemistry linkers used in the invention are discussed in Examples 8 and 9.

### Example 6

### SATA Chemical Linkage

In another example the targeting moiety is joined to the lysosomal enzyme through an SATA chemical linker. An exemplary scheme for generating such a conjugate is shown below.

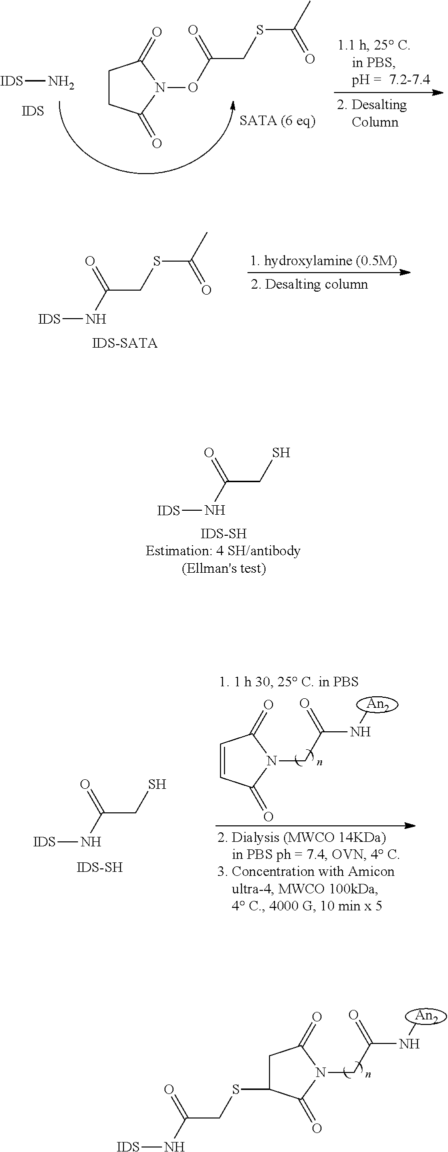

### Example 7

### Other Chemical Conjugation Strategies

In another example, chemical conjugation is achieved through a hydrazide linker. An exemplary scheme for generation of such a conjugate is as follows.

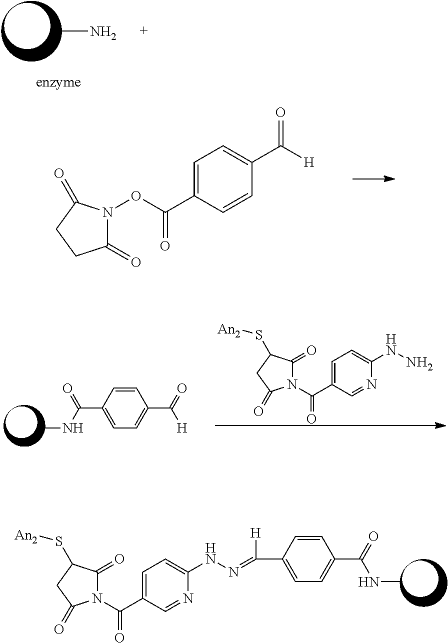

In another example, chemical conjugation is achieved using a periodate-oxidated enzyme with a hydrazide derivative through a sugar moiety (e.g., a glycosylation site). An example of this approach is shown below using a protected-propionyl hydrazide.

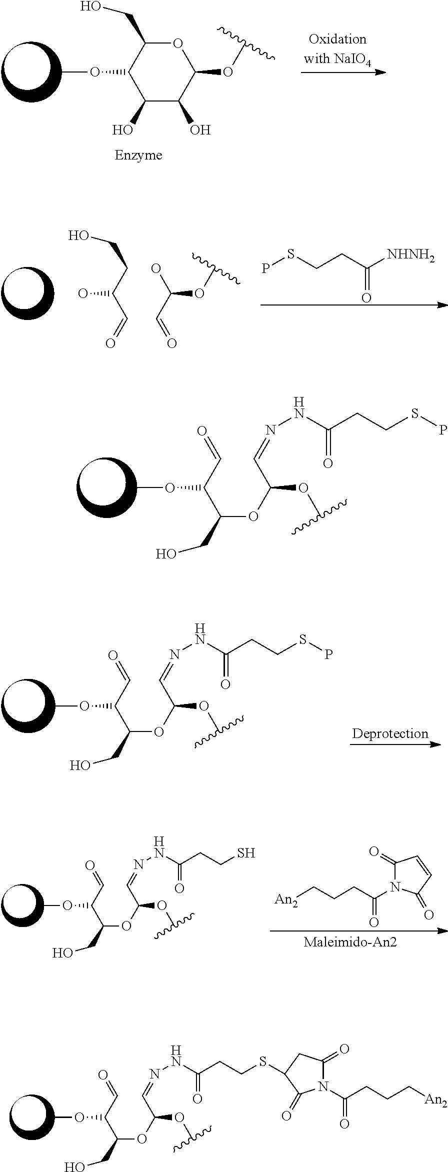

Another example of this approach is shown below.

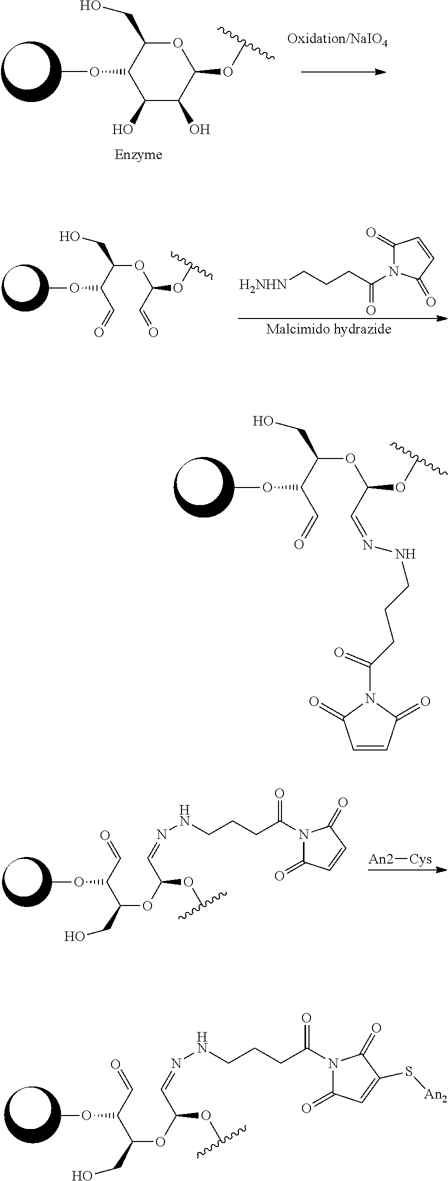

### Example 8

### Methods for Conjugation of IDS with An2 by Click Chemistry

Possible Conjugation Sites in the Amino Acid Sequence of Iduronate-2-Sulfatase Include the Lysine and N-Terminal Residues.

```
                10 20 30 40
MPPPRTGRGL LWLGLVLSSV CVALGSETQA NSTTDALNVL
                50 60 70 80
LIIVDDLRPS LGCYGDKLVR SPNIDQLASH SLLFQNAFAQ
                90 100 110 120
QAVCAPSRVS FLTGRRPDTT RLYDFNSYWR VHAGNFSTIP
              130 140 150 160
QYFKENGYVT MSVGKVFHPG ISSNHTDDSP YSWSFPPYHP
              170 180 190 200
SSEKYENTKT CRGPDGELHA NLLCPVDVLD VPEGTLPDKQ
              210 220 230 240
STEQAIQLLE KMKTSASPFF LAVGYHKPHI PFRYPKEFQK
              250 260 270 280
LYPLENITLA PDPEVPDGLP PVAYNPWMDI RQREDVQALN
              290 300 310 320
ISVPYGPIPV DFQRKIRQSY FASVSYLDTQ VGRLLSALDD
              330 340 350 360
LQLANSTIIA FTSDHGWALG EHGEWAKYSN FDVATHVPLI
              370 380 390 400
FYVPGRTASL PEAGEKLFPY LDPFDSASQL MEPGRQSMDL
              410 420 430 440
VELVSLFPTL AGLAGLQVPP RCPVPSFHVE LCREGKNLLK
              450 460 470 480
HFRFRDLEED PYLPGNPREL IAYSQYPRPS DIPQWNSDKP
              490 500 510 520
SLKDIKIMGY SIRTIDYRYT VWVGFNPDEF LANFSDIHAG
              530 540 550
ELYFVDSDPL QDHNMYNDSQ GGDLFQLLMP
```

### Compound Structures

Angiopep2 Sequence

### H₂N-Thr-Phe-Phe-Tyr-Gly-Gly-Ser-Arg-Gly-Lys-Arg-Asn-Asn-Phe-Lys-Thr-Glu-Glu-Tyr-COOH

Azido-An2 (N-Terminus) 4

The structure of Azidobutyryl-An2 (Azido-An2) with an N-terminal azide group is shown below. This compound was made by standard solid phase synthesis methods.

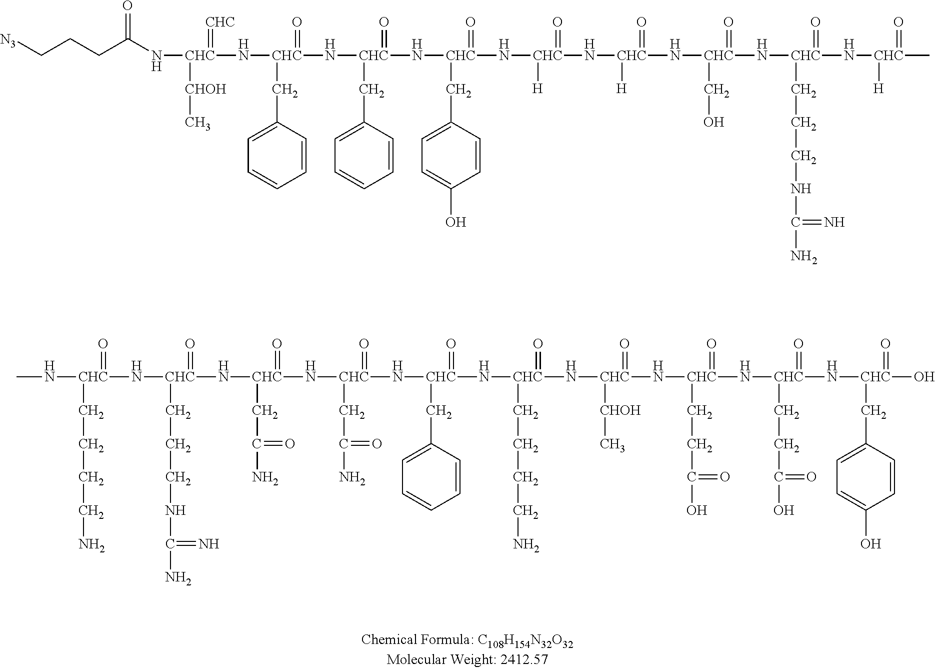

An2-Azido (C-Terminus) 8

The structure of An₂-[azido-norleucine](An2-Azido) with a C-terminal azide group is shown below. This compound was made by standard solid phase synthesis methods.

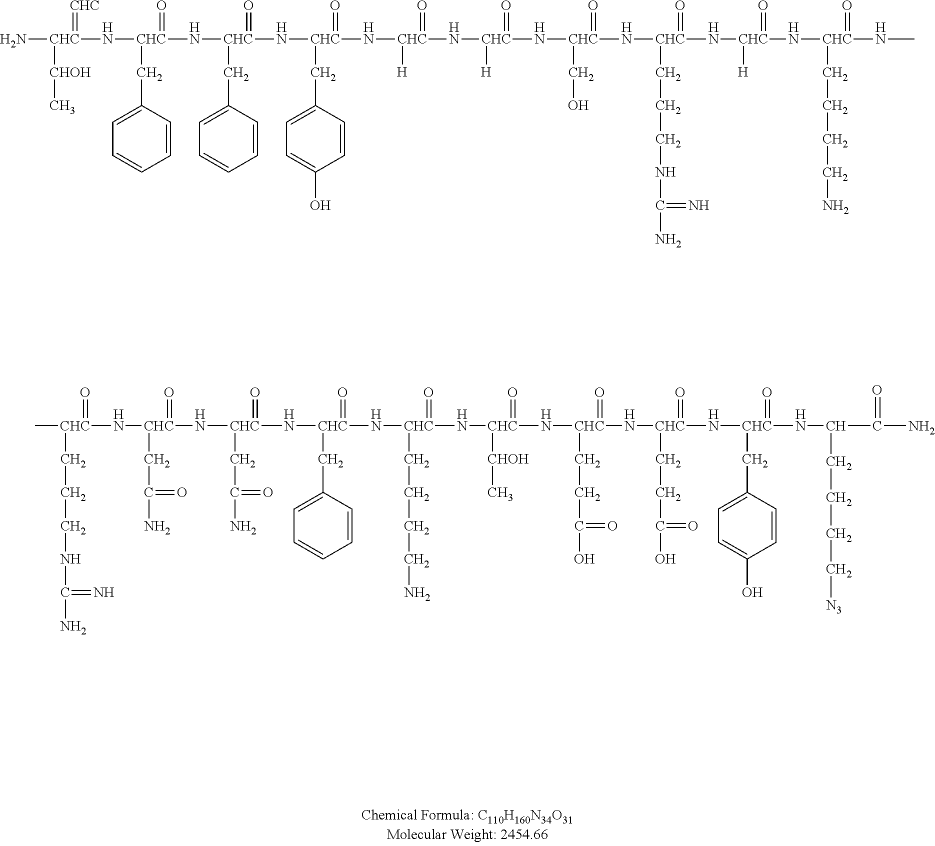

### Schematic Structure:

The structure of IDS-BCN-Butyryl-An₂ (70-56-1B and 70-56-2B) is shown below.

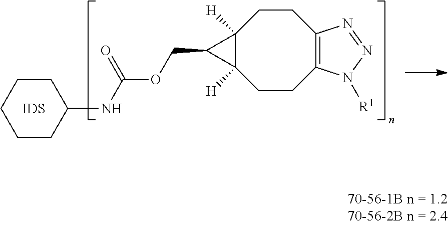

wherein R¹ is:

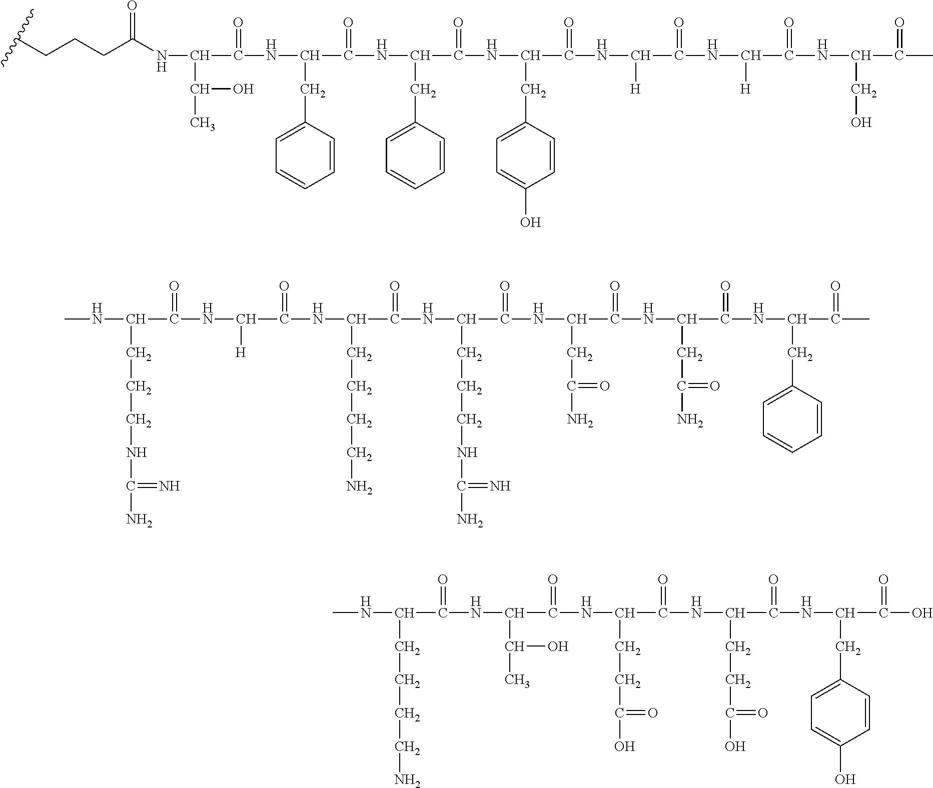

wherein the NH group attached to IDS is derived from the reaction of a primary amino group in IDS.

The structure of An₂-[azido-norleucine]-MFCO-IDS (70-66-1B) is shown below.

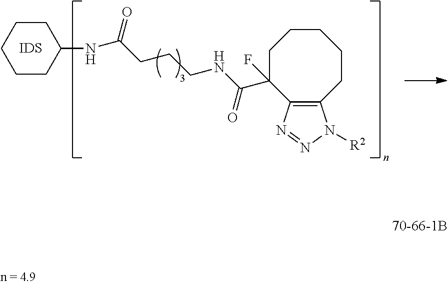

wherein R2 is:

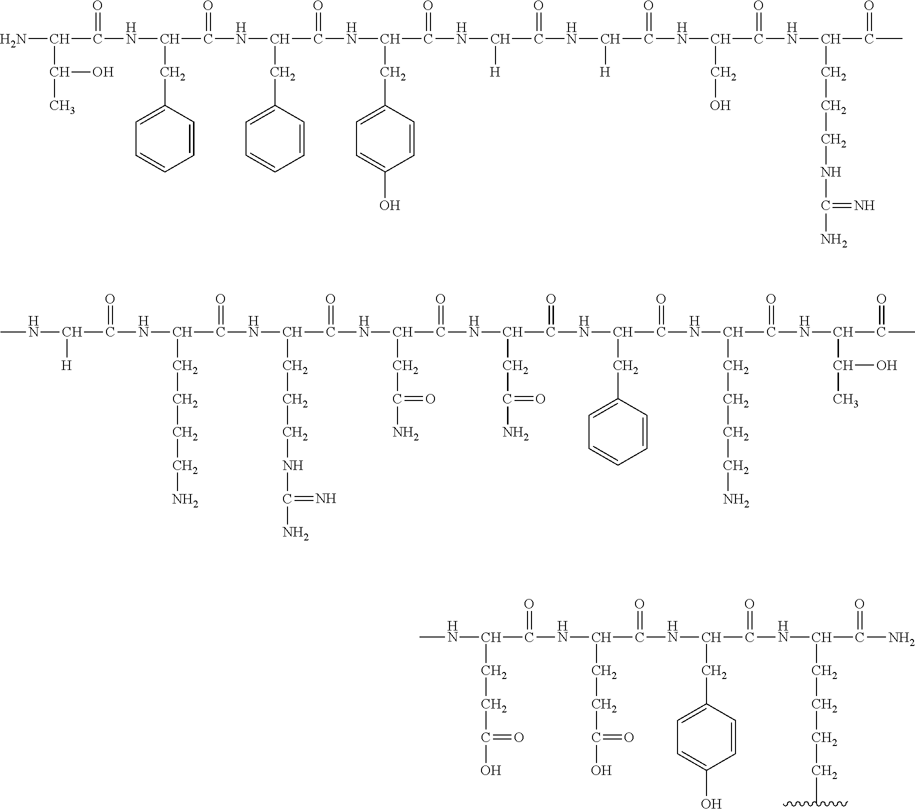

wherein the NH group attached to IDS is derived from the reaction of a primary amino group in IDS.

The structure of An₂-[azido-norleucine]-BCN-IDS (68-32-2) is shown below.

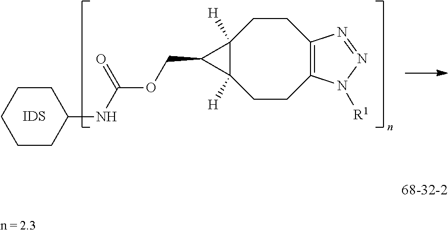

wherein R¹ is:

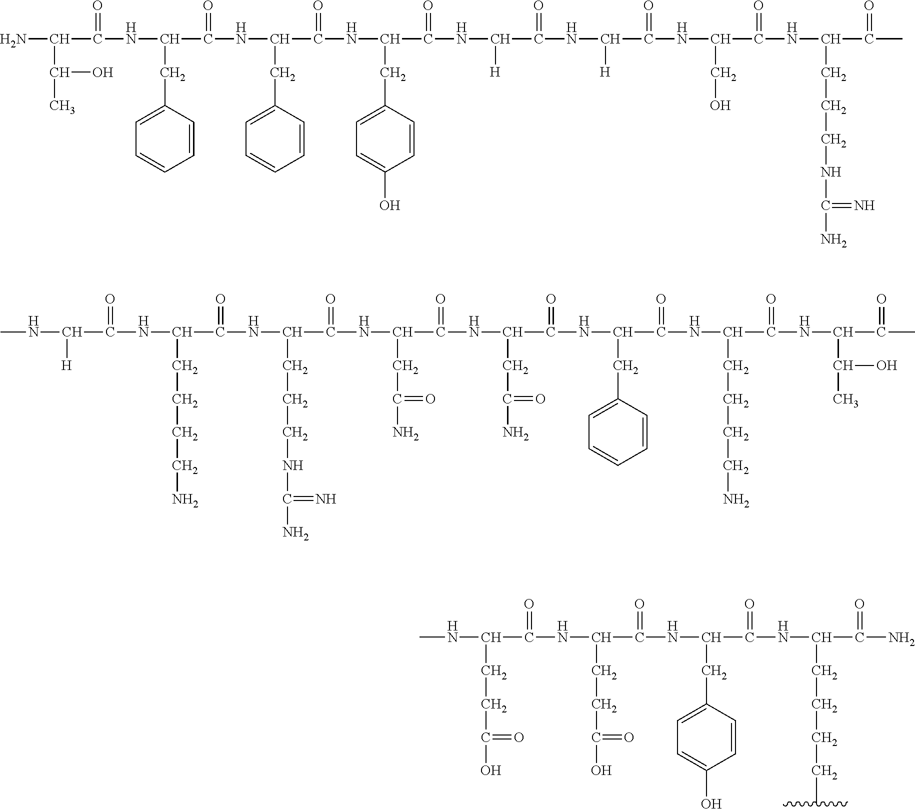

wherein the NH group attached to IDS is derived from the reaction of a primary amino group in IDS.

### Synthesis Scheme for 70-56-1B and 70-56-2B

### Step: 1

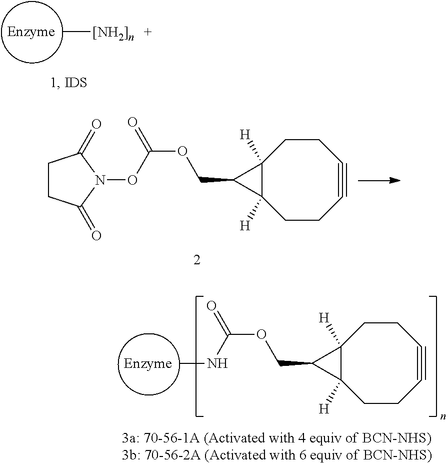

### Step: 2

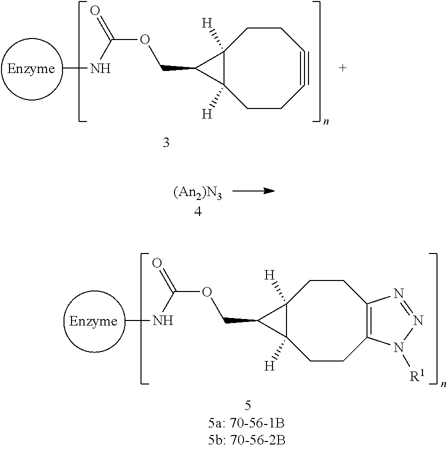

wherein R¹ is:

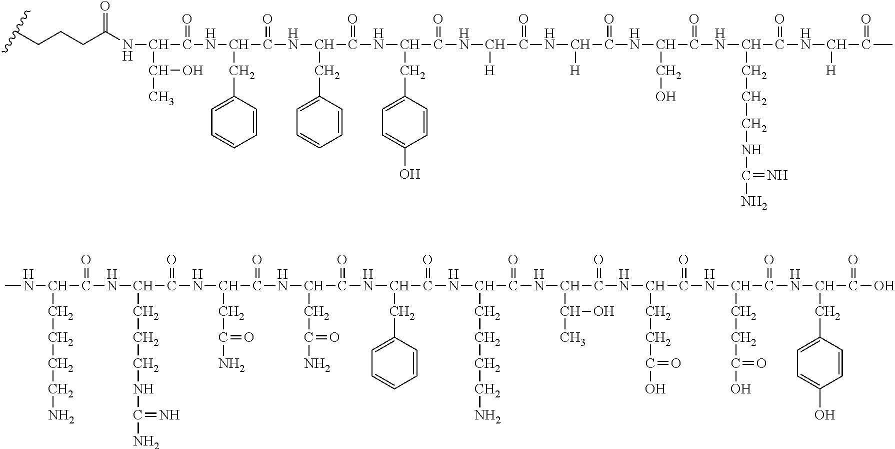

BCN: bicyclo[6.1.0]nonyne

### Synthesis of 70-56-1A

To (7.24 mg, 95 nmole) of IDS (1) in phosphate buffer 20 mM at pH ˜7.6, 380 nmole (4 equiv) of the BCN-N-hydroxysuccinimide ester (2) (from stock solution prepared as follows: 5.82 mg dissolved in 1000 μl of anhydrous DMSO) was added at RT for 5 h with occasional manual shaking. The modified IDS 3a, 70-56-1A was purified from the excess reagent by gel filtration with HiPrep 26/10 desalting column at 5 mL/minute with phosphate buffer 20 mM pH 7.6. The collected fractions were concentrated by Amicon ultra centrifugal filter (limit 10 kDa, 3000 rpm) to 3.8 mL (6.5 mg, yield 90%). The modified IDS 70-56-1A (3a) was recovered and was used for the next conjugation step with azido-An2 (N-terminus) (4).

Step: 2—Conjugation of Modified IDS with Azido-An2 (N Terminus)

### Synthesis of (70-56-1B)

To modified IDS derivative (3a) (6.5 mg, 85.2 nmole), 8 equiv of azido-An2 (N-terminus) (4) was added. The solution was manually shaken, wrapped on aluminum foil and left overnight at RT. The conjugate (5) was then purified by Q Sepharose 1 mL column using 20 mM TRIS at pH7 as binding buffer whereas 20 mM TRIS and 500 mM NaCl at pH 7.0 was used as eluent buffer. The conjugate was isolated and was exchanged with IDS buffer (1×: 137 mM NaCl, 17 mM NaH₂PO₄, 3 mM Na₂HPO₄, at pH ˜6) by washing 5 times 15 mL with Amicon ultra centrifugal filter (10 kDa cut-off, 3000 rpm) and was concentrated to 2.5 mL to obtain 70-56-1B (6 mg, yield 83%).

### Synthesis of 70-56-2A

To 7.24 mg (95 nmole) of IDS (1) in phosphate buffer 20 mM at pH-7.6, 570 nmole (6 equiv) of the BCN-N-hydroxysuccinimide ester (2) was added at RT for 5 h with occasional manual shaking. The activated IDS 70-56-2B (3b) was purified from the excess reagent by gel filtration with HiPrep 26/10 desalting column at 5 mL/minute with phosphate buffer 20 mM pH 7.6. The collected fractions were concentrated by Amicon ultra centrifugal filter (10 kDa, 3000 rpm) to 3.5 mL, (6.5 mg, yield 90%). The modified IDS 3b, 70-66-2A was recovered which was used for the next conjugation step with azido-An2 (N-terminus) (4).

### Synthesis of (70-56-2B)

To modified IDS 3b, 70-56-2A (6.5 mg, 85.2 nmole), 12 equiv of azido-An2 (N-terminus) (4) were added. The solution was manually shaken and wrapped on aluminum foil and left overnight at RT. The conjugate (5) was purified by Q Sepharose 1 mL column using 20 mM TRIS buffer at pH 7 as binding buffer and 20 mM TRIS and 500 mM NaCl at pH 7.0 was used as eluent buffer. The conjugate was isolated and was exchanged with IDS buffer (1×: 137 mM NaCl, 17 mM NaH₂PO₄, 3 mM Na₂HPO₄, at pH-6) by washing 5 times 15 mL with Amicon ultra centrifugal filter (10 kDa limit, 3000 rpm) and was concentrated to 3 mL to obtain 70-56-2B (6 mg, 83%).

### Large Scale Synthesis of 70-56-2B

To (152 mg, 1992 nmole) of IDS in Phosphate buffer 20 mM at pH 7.6, 11952 nmole (6 equiv) of the BCN-N-hydroxysuccinimide ester (20 mmolar in anhydrous DMSO) was added at RT for 5 h with occasional manual shaking. The modified IDS 70-56-2B was purified from the excess reagent by gel filtration with desalting column Sephadex G25 at 15 mL/minute with phosphate buffer 20 mM, pH 7. The collected fractions (65 mL) were concentrated by Centricon Plus-70 (10 kDa, 3100 rpm) centrifugal filters to 36 mL, (145 mg, yield 95%). The modified IDS was recovered for the next conjugation step with azido An2 (N-terminus). To modified IDS (130 mg, 1704 nmole), 12 equiv of azidoAn2 (62 mg) were added. The solution was manually shaken, wrapped with aluminum foil and left overnight at RT. Conjugate was purified on a Q Sepharose 20 mL column using 20 mM TRIS buffer at pH 7 for binding buffer and 20 mM TRIS with 500 mM NaCl at pH 7.0 for eluent buffer. Conjugate (90 mL) was isolated, concentrated to 30 mL using Centricon Plus-70 (10 kDa, 3100 rpm) and exchanged with IDS buffer (1×: 137 mM NaCl, 17 mM NaH₂PO₄, 3 mM Na₂HPO₄, at pH 6) by washing (4×35 mL IDS buffer). The material was then concentrated to 33 mL with Centricon Plus-70 (10 kDa, 3100 rpm) columns and sterile filtered to obtain 70-56-2B (114 mg, 88%).

### Synthesis Scheme for 70-66-1B

The synthesis scheme shown below shows the attachment of a linker containing MFCO linker to IDS and attachment of An₂-[azido-norleucine](An2-azido (C-terminus)) to the linker containing MFCO via the azido group of An2-azido (C-terminus) 8.

### Synthesis Scheme for 70-66-1B

### Step: 1

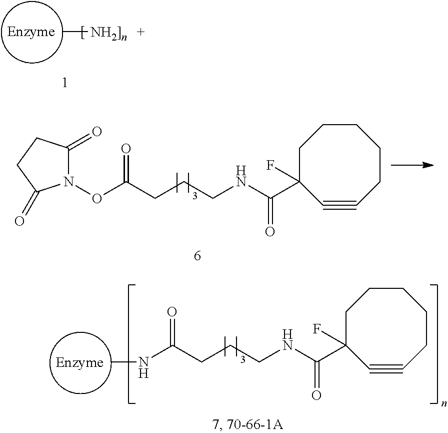

### Step: 2

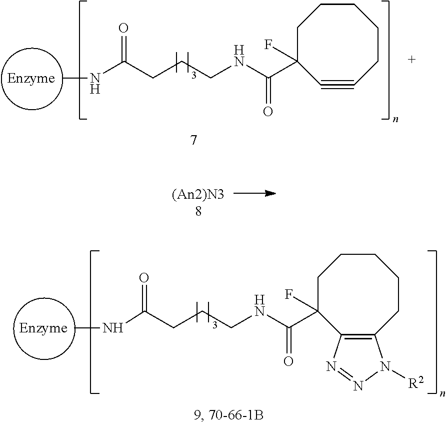

wherein R² is:

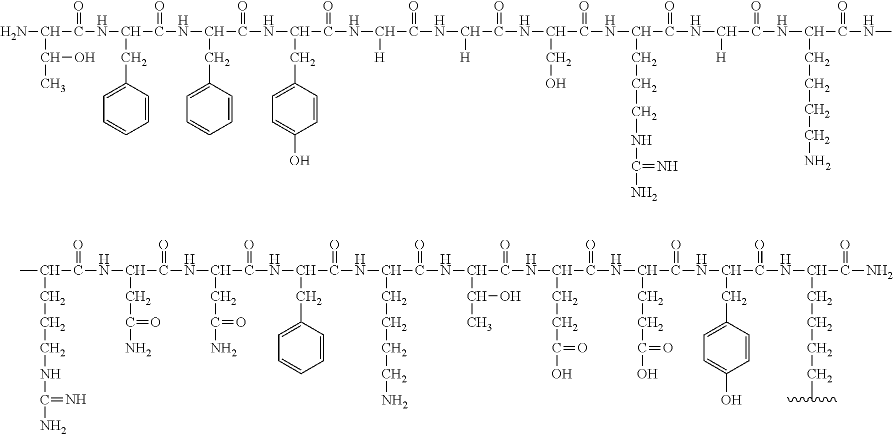

### MFCO: Monofluorocyclooctyne

### Synthesis of 70-66-1A

To (10.6 mg, 139 nmole) of IDS (1) in phosphate buffer 20 mM at pH-7.6, 1112 nmole (8 equiv) of the MFCO-N-hydroxysuccinimide ester (6) (from stock solution prepared as follows: 7.6 mg dissolved in 1000 μl of anhydrous DMSO) was added and was left at RT for 5 h with occasional manual shaking. The modified IDS 70-66-1A (7) was purified from the excess reagent by gel filtration with HiPrep 26/10 desalting column at 5 mL/minute with phosphate buffer 20 mM pH 7.6. The collected fractions were concentrated by Amicon ultra centrifugal filter (10 kDa limit, 3000 rpm) to 3 mL, (9.4 mg, yield 89%). The modified IDS (7) was used for the next conjugation step with An2-azido (C-Terminus) (8).

Step: 2—Conjugation of Modified IDS with An2-Azido (C Terminus) (An₂-[Azido-Norleucine])

To modified IDS derivative (7), (6.1 mg, 80 nmole), 16 equiv of An2-azido (C-terminus) (8) were added. The solution was manually shaken and wrapped on aluminum foil and left overnight at RT. The conjugate (9) was purified by Q Sepharose 1 mL column using 20 mM TRIS at pH 7 as binding buffer whereas 20 mM TRIS and 500 mM NaCl at pH 7.0 was used as eluent buffer. The conjugate was isolated and was exchanged with IDS buffer (1×: 137 mM NaCl, 17 mM NaH₂PO₄, 3 mM Na₂HPO₄ at pH-6) by washing 5 times 15 mL with Amicon ultra centrifugal filter (10 K mW, 3000 rpm) and was concentrated to 2.5 mL to obtain 70-66-1B (6.1 mg, 100%).

### Large Scale Synthesis of 70-66-1B

To IDS (152 mg, 1992 nmole) in phosphate buffer 20 mM at pH 7.6, 15936 nmole (8 equiv) of the MFCO-N-hydroxysuccinimide ester (20 mMr in anhydrous DMSO) was added at RT for 5 h with occasional manual shaking. The modified IDS was purified from the excess reagent by gel filtration with desalting column Sephadex G25 at 15 mL/minute with phosphate buffer 20 mM pH 7. The collected fractions (65 mL) were concentrated by Centricon Plus-70 (10 kDa, 3100 rpm) centrifugal filters to 36 mL, (157 mg, yield ˜100%). The modified IDS was recovered for the next conjugation step with An2 azido (C-terminus).

To modified IDS (130 mg, 1704 nmole), solution of 16 equiv of An2-azido (C-terminus) (84 mg, 10 mg/mL in deionized water) were added. Solution was manually shaken, wrapped on aluminum foil and left overnight at RT. Conjugate was purified on a Q Sepharose 20 mL column using 20 mM TRIS buffer at pH 7 for binding buffer and 20 mM TRIS with 500 mM NaCl at pH 7.0 for eluent buffer. Conjugate (90 mL) was isolated, concentrated to 30 mL using Centricon Plus-70 (10 kDa, 3100 rpm) and exchanged with IDS buffer (1×: 137 mM NaCl, 17 mM NaH₂PO₄, 3 mM Na₂HPO₄, at pH 6) by washing (4×35 mL IDS buffer). The material was then concentrated to 33 mL with Centricon Plus-70 (10 kDa, 3100 rpm) columns and sterile filtered to obtain 70-66-1B (104 mg, 80% yield).

### Synthesis Scheme for 68-32-2

### Step: 1

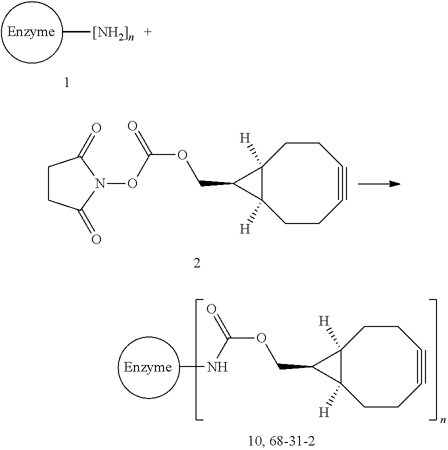

### Step: 2

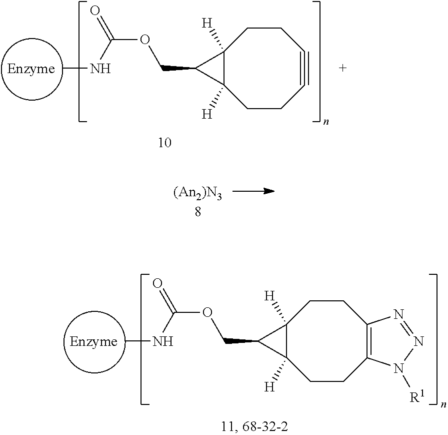

wherein R¹ is:

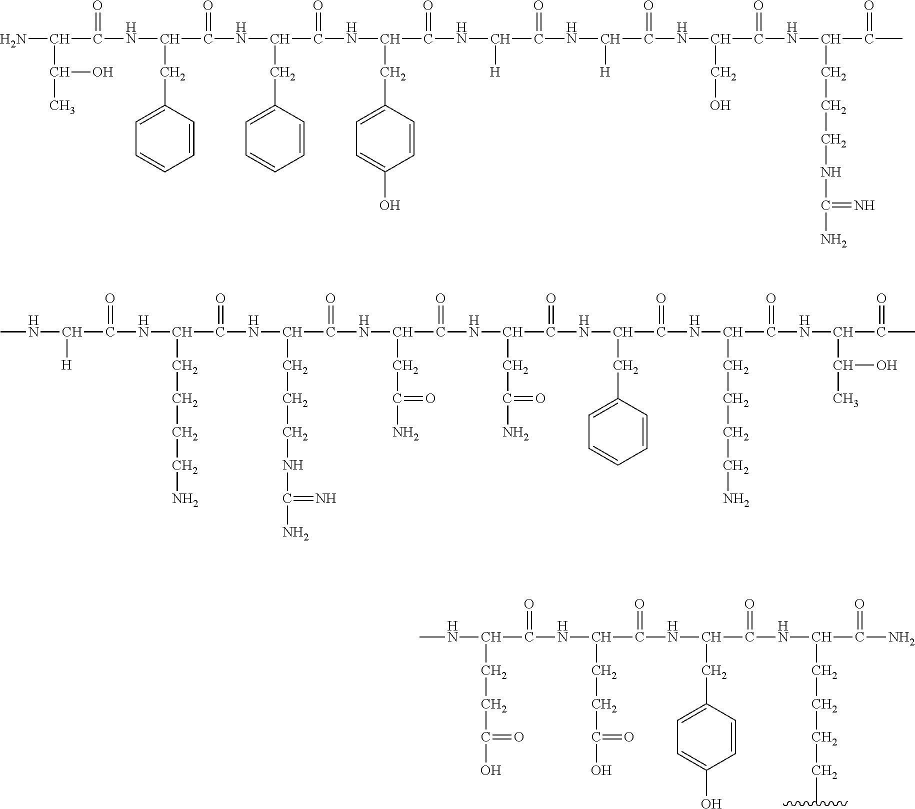

BCN: bicyclo[6.1.0]nonyne

### Synthesis of 68-31-2

To (14.5 mg, 190 nmole) of IDS (1) in phosphate buffer 20 mM at pH ˜7.6, 1520 nmole (8 equiv) of the BCN-N-hydroxysuccinimide ester (2) (from stock solution prepared as follows: 5.82 mg dissolved in 1000 μl of anhydrous DMSO) was added and stored at RT for 5 h with occasional manual shaking. The modified IDS (10) was purified from the excess reagent by gel filtration with HiPrep 26/10 desalting column at 5 mL/minute with phosphate buffer 20 mM pH 7. The collected fractions were concentrated by Amicon ultra centrifugal filter (limit 10 kDa, 3000 rpm) to 4 mL (14.5 mg, yield 100%). The modified IDS was recovered and was used for the next conjugation step with An2-azido (C-terminus).

Step: 2—Conjugation of Modified IDS with An2-Azido (C Terminus) (An₂-[Azido-Norleucine])

### Synthesis of 68-32-2

To modified IDS derivative (10) (11 mg, 144.2 nmole), 16 equiv of An2-azido (C-terminus) were added. The solution was manually shaken and wrapped on aluminum foil and left overnight at RT. The conjugate (11) was purified by Q Sepharose 1 mL column using 20 mM TRIS at pH 7 as binding buffer where as 20 mM TRIS and 500 mM NaCl at pH 7.0 was used as eluent buffer. The conjugate was isolated and was exchanged with IDS buffer (1×: 137 mM NaCl, 17 mM NaH₂PO₄, 3 mM Na₂HPO₄ at pH ˜6) by washing 5 times 15 mL with Amicon ultra centrifugal filter (10 K mW, 3000 rpm) and was concentrated to 2.5 mL to obtain 68-32-2 (10 mg, 91%).

### Large Scale Synthesis of 68-32-2

To (152 mg, 1992 nmole) of IDS in Phosphate buffer 20 mM at pH ˜7.6, 15936 nmole (8 equiv) of the BCN-N-hydroxysuccinimide ester (from stock solution prepared as follows: 5.82 mg dissolved in 1000 μl of anhydrous DMSO) was added at RT for 5 h with occasional manual shaking. The modified IDS) was purified from the excess reagent by gel filtration with desalting column Sephadex G25 at 15 mL/minute with phosphate buffer 20 mM ˜pH 7. The collected fractions 65 mL were concentrated by Centricon Plus-70 (10 kDa, 3100 rpm) centrifugal filter to 36 mL (145 mg, yield ˜95%). The modified IDS was recovered which was used for the next conjugation step with An2 azido (C-terminus).

Solutions of 16 equiv of azidoAn2 (C-terminus) (84 mg)(10 mg/mL in deionized water) were added to modified IDS (130 mg, 1704 nmole). The solution was manually shaken and wrapped on aluminum foil and left overnight at RT. Conjugate was purified on a Q Sepharose 20 mL column using 20 mM TRIS buffer at pH 7 for binding buffer and 20 mM TRIS with 500 mM NaCl at pH 7.0 for eluent buffer. Conjugate (90 mL) was isolated, concentrated to 30 mL using Centricon Plus-70 (10 kDa, 3100 rpm) and exchanged with IDS buffer (1×: 137 mM NaCl, 17 mM NaH₂PO₄, 3 mM Na₂HPO₄, at pH 6) by washing (4×35 mL IDS buffer). The material was then concentrated to 33 mL with Centricon Plus-70 (10 kDa, 3100 rpm) columns and sterile filtered to obtain 68-32-2 (104 mg, 80% yield).

### Example 9

### Synthesis of IDS-Angiopep-2 Conjugates with Cleavable Linkers

An2 is conjugated to IDS via a disulfide containing cleavable linker via the two schemes shown below. In the first scheme the lysine side chain of IDS is reacted with a SPDP linker to generate modified IDS. The modified IDS is reacted with An₂-Cys-SH to attach the An2 via the S moiety of the C-terminal cysteine of An₂-Cys to generate an IDS-An₂ conjugate.

In the second scheme, IDS is reacted with a SATA linker followed by reaction with hydroxylamine to generate modified IDS. The N-terminus of An₂ is reacted with SPDP to generate a modified An₂. The modified IDS is reacted with the modified An₂ to attach the An₂, via the N-terminal amino group of An₂, to IDS to generate a IDS-An₂ conjugate.

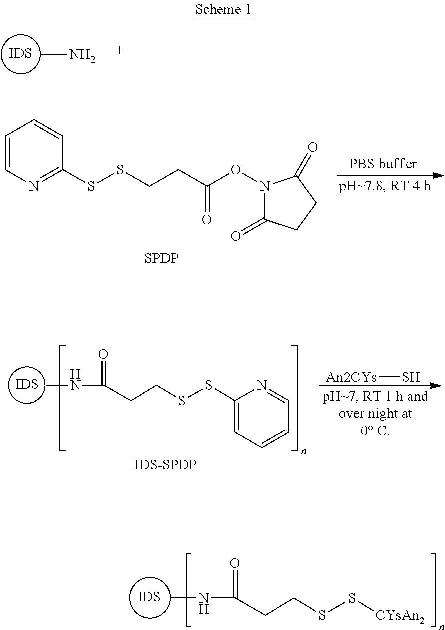

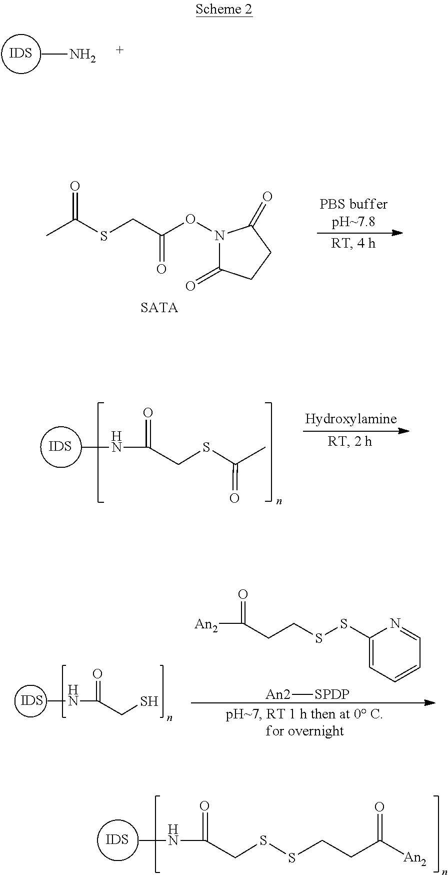

### Example 10

### Screening and Characterization of Compounds

Recombinant iduronate-2-sulfatase (IDS) (JR-032) was conjugated to An2 via lysine attachment. The IDS amino acid sequence with potential attachment sites marked is presented above in Example 8. These conjugates represent varying ratios of An2:linker to IDS. Linkers tested in this conjugation strategy were click chemistry linkers including MFCO (monofluorocyclooctyne), BCN (bicyclononyne), SATA (S-acetylthioacetate), DBCO (dibenzylcyclooctyne), and maleimido. In all cases, the ratio of An2:linker material added to the reaction is 2:1, with An2 in excess of IDS by either 4-, 6-, or 8-fold. An2 was removed from the reaction product by Q-sepharose column chromatography, and MALDI-TOF analysis was used to determine the average number of An2 incorporated on each IDS. SP-HPLC analysis was used to determine whether unconjugated IDS was present in the product. SEC analysis was used to examine the quality of the protein following conjugation. Using this method, the first series of nine conjugates were found to have evidence of aggregate formation, and the conjugation reactions were optimized and repeated to eliminate this issue. In addition, five novel conjugates were produced using other linkers.

As shown below in Table 3, these conjugates were evaluated to determine:

1. An2 incorporation (range of 1-5 An2/IDS)

2. degree of aggregation by size exclusion chromatography (SEC)

3. major peaks by side population (SP)-analysis

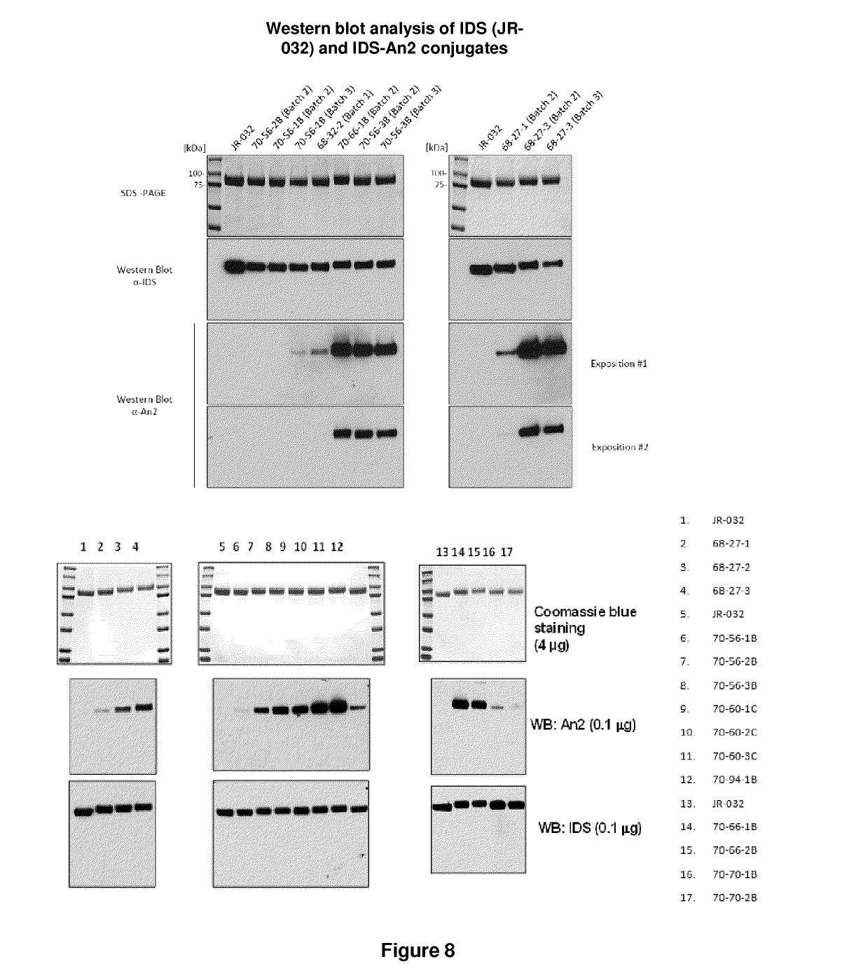

The lysine conjugates that were selected for further analysis are presented in Table 3 below. Note that the number of An2 incorporated is an average, as multiple species may exist in conjugation reaction products. The mass of JR-032 by MALDI TOF is 76,320 Da (11 determinations). Western blots for these conjugates are presented in FIG. 8.

### Uptake of Alexa488-IDS and Alexa488-An2-IDS (70-56-2B) in U87 Cells.

U87 cells were seeded in 12-wells plates and allowed to grow for 48 h in normal cell culture conditions. Cellular uptake experiments were performed by incubating the confluent U87 cells with increasing concentrations of Alexa488-labelled products in complete cell culture media (without phenol red and with antibiotics) for 1 h or 16 h in cell culture conditions. Cells were then washed once, trypsinysed and extensively washed again on ice. Uptake of the fluorescent compound in cell was evaluated by flow cytometry. Results were expressed as the relative fluorescence units (RFU) of samples after subtracting the basal cell fluorescence measured in absence of labelled compounds.

TABLE 3
An2-IDS lysine conjugates selected for further analysis.

| | | | | MW of | Mass of | | | |
|---|---|---|---|---|---|---|---|---|
| IDS-An2 | | | Ratio | linker + | Conjugate | Number of An2 | Yield | |
| Conjugate | Linker | An2 | (Activation:An2) | An2 | By Maldi Tof | Incorporated | (%) | Code (Name) |
| 68-27-1 | MFCO | An2 | 4:8 | 2678 | 83,362 | ~2.3¹ | 80 | |
| | | | | | | (2.6; 2.0) | | |
| 68-27-2 | MFCO | An2 | 6:12 | 2678 | 88,133 | 4.4 | 65 | |
| 68-27-3 | MFCO | An2 | 8:16 | 2678 | 90,484 | ~5.0² | 65 | |
| | | | | | | (5.3; 4.2; 5.5) | | |
| 70-56-1B | BCN | An2 | 4:8 | 2589 | 79,265 | ~1.2² | 83 | |
| | | | | | | (1.2; 1.0; 1.2) | | |
| 70-56-2B | BCN | An2 | 6:12 | 2589 | 81,321 | ~2.4¹ | 81 | ANG3401 |
| | | | | | | (2.0; 2.8) | | (IDS-BCN- |
| | | | | | | | | Butyryl-An₂) |
| 70-56-3B | BCN | An2 | 8:16 | 2589 | 82,826 | ~3.0² | 80 | |
| | | | | | | (2.5; 3.2; 3.3) | | |
| 70-60-1C | SATA | An2 | 4:8 | 2570 | 80,303 | 1.5 | 84 | |
| 70-60-2C | SATA | An2 | 6:12 | 2570 | 82,961 | 2.6 | 80 | |
| 70-60-3C | SATA | An2 | 8:16 | 2570 | 85,289 | 3.5 | 81 | |
| 70-066-1B | MFCO | An2N3 (C) | 8:16 | 2719 | 89,566 | ~4.9¹ | 100 | ANG3402 |
| | | | | | | (4.9; 4.8) | | (An₂-[azido- |
| | | | | | | | | norleucine]- |
| | | | | | | | | MFCO-IDS) |
| 70-066-2B | MFCO | An2N3 (N) | 8:16 | 2678 | 89,374 | 4.9 | 93 | |
| 70-070-1B | Maleimide | An2Cys (C) | 8:16 | 2675 | 78,562 | 0.8 | 100 | |
| 70-070-2B | Maleimide | An2Cys (N) | 8:16 | 2675 | 78,773 | 0.9 | 100 | |
| 70-094-1B | DBCO | An2N3 (N) | 8:16 | 2728 | 79,840 | 1.3 | 100 | |
| 68-32-2 | BCN | An2N3 (C) | 8:16 | 2631 | 83,738 | 2.3 | TBD | ANG3403 |
| | | | | | | | | (An₂-[azido- |
| | | | | | | | | norleucine]- |
| | | | | | | | | BCN-IDS) |

4. ¹=average of two values.
5. ²=average of three values.

A cysteine strategy was also employed in an effort to limit (and standardize) the number of An2 incorporated to one per IDS, however, no more than 50% of IDS conjugation with An2 was attained using a range of conditions including up to 20 equivalents of An2. Moreover, the conjugation reaction products showed a 50% loss of enzymatic activity, suggesting that the conjugated material was inactive. Thus, the lysine approach was favored.

### Results

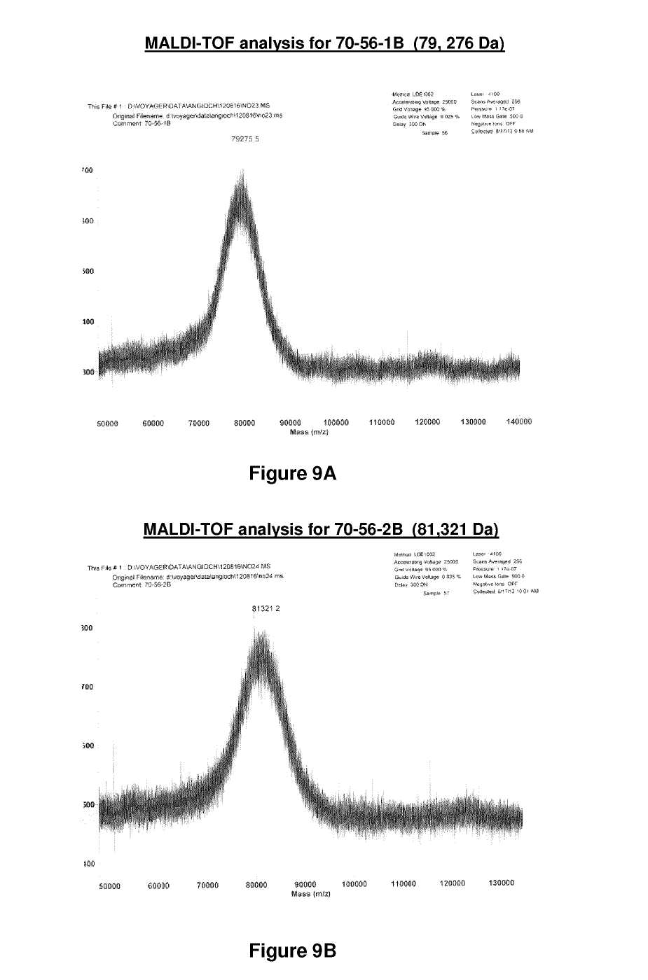

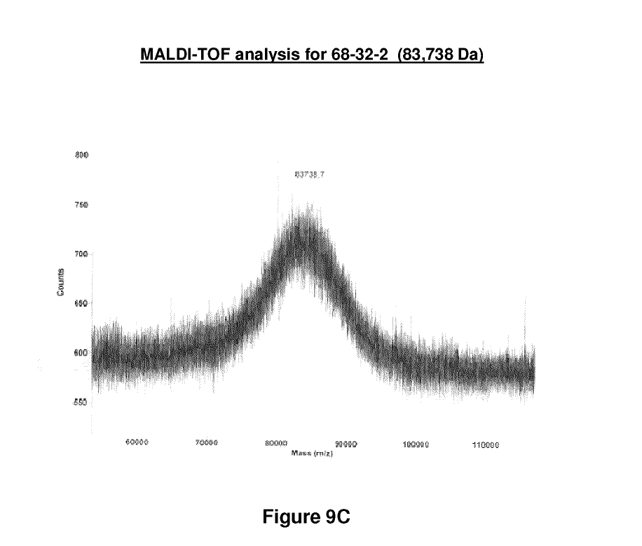

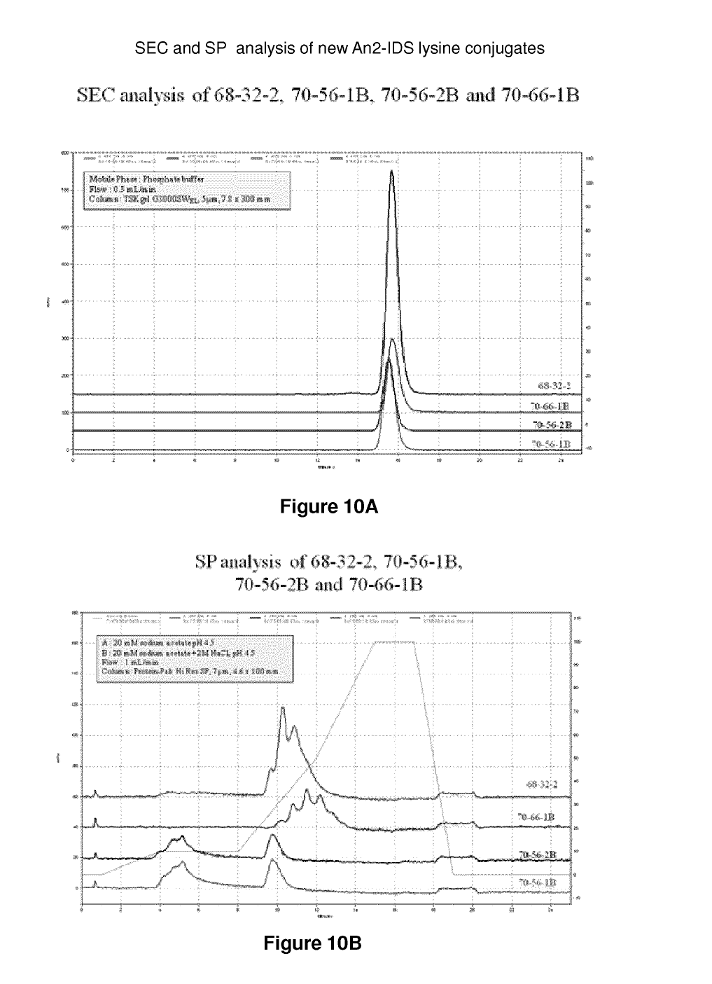

FIGS. 9A, 9B, 9C, and 9D show MALDI-TOF analyses of 70-56-1B, 70-56-2B, 68-32-2, and 70-66-1B respectively. FIGS. 10A and 10B show SEC and SP analyses of 68-32-2, 70-56-1B, 70-56-2B, and 70-66-1B. The structures of these conjugates and a summary of the synthetic protocols are provided above. The average numbers of An2 incorporated into 68-32-2, 70-66-1B, 70-56-2B, and 70-56-1B are 2.3, 4.9, 2.4, and 1.2, respectively. No unconjugated JR-032 is detected in these analyses. Two peaks, representing two populations of An2-IDS, are visible for each conjugate, one eluting at 4-5 minutes and the second at 10 minutes. Purification of similarly spaced peaks for a different An2-IDS conjugate has been demonstrated.

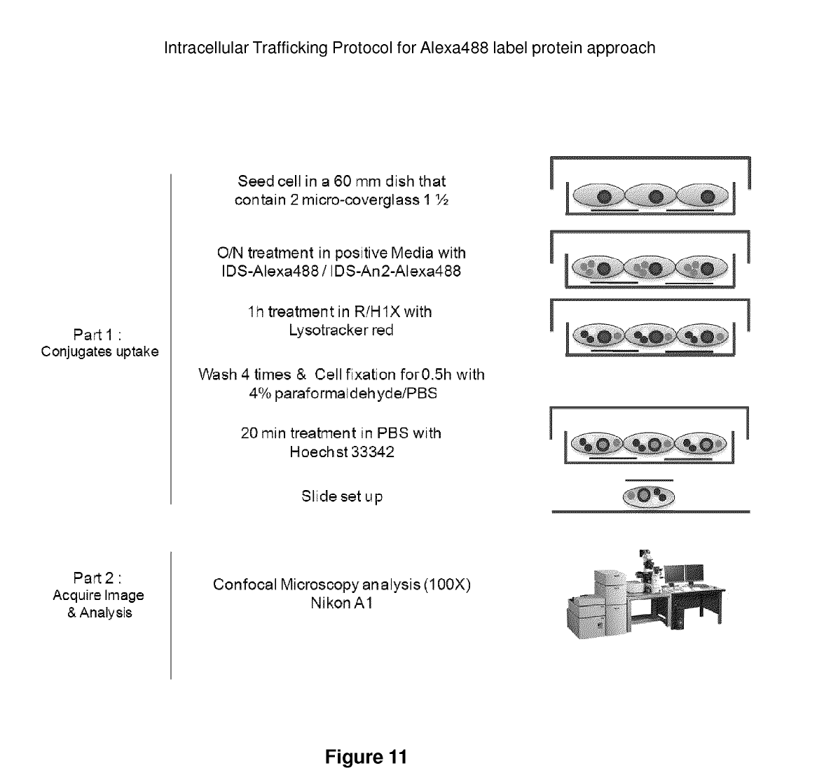

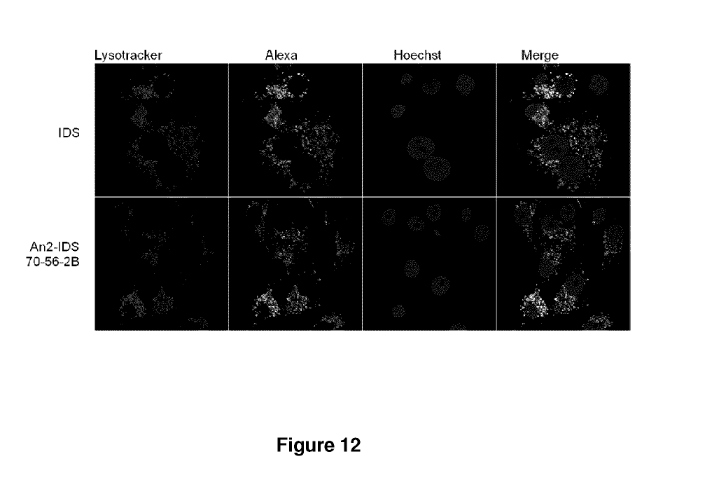

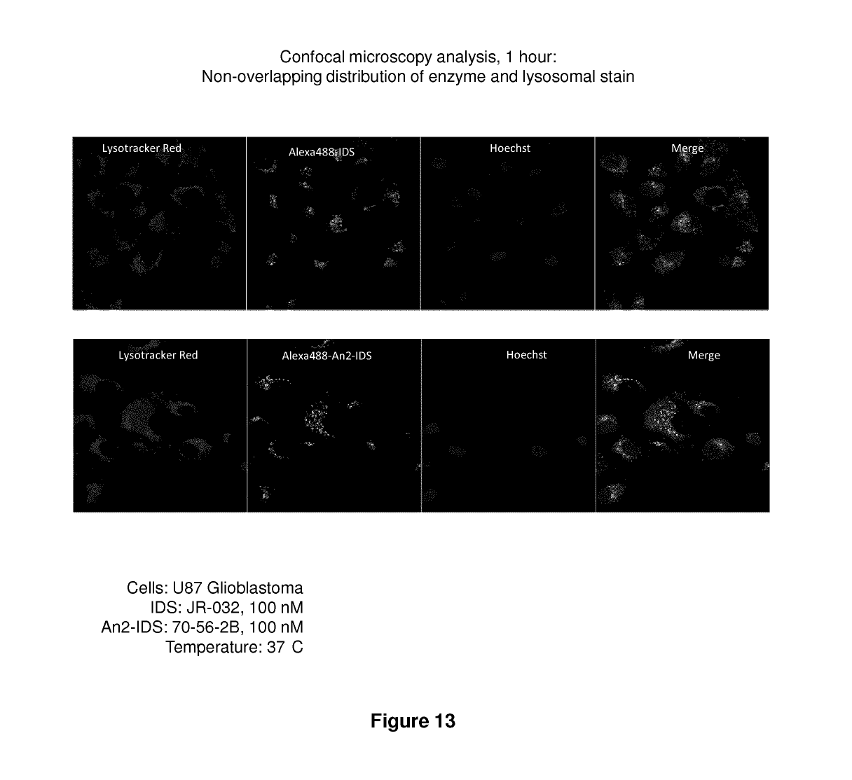

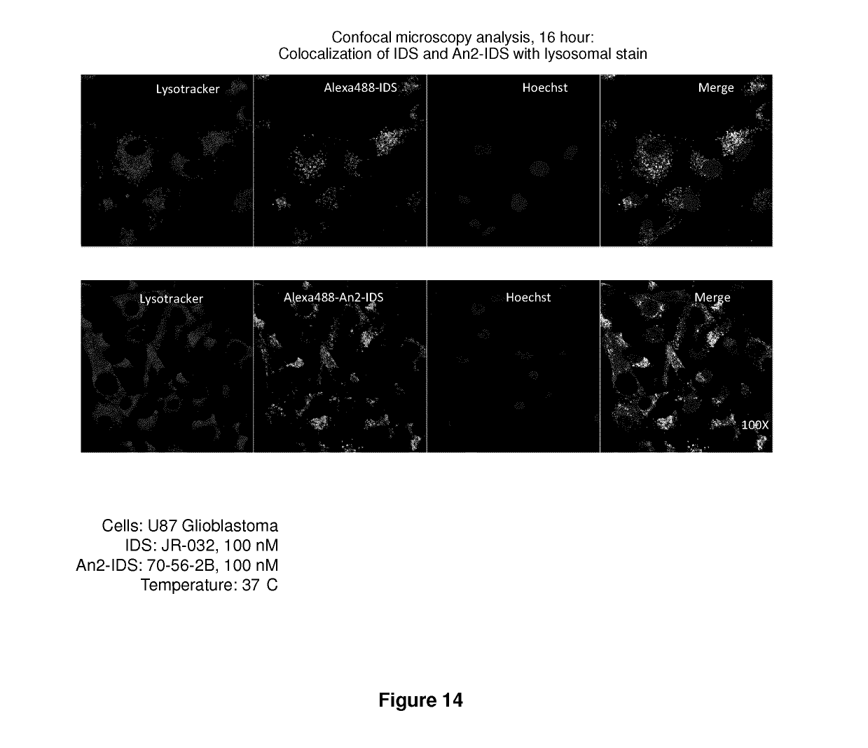

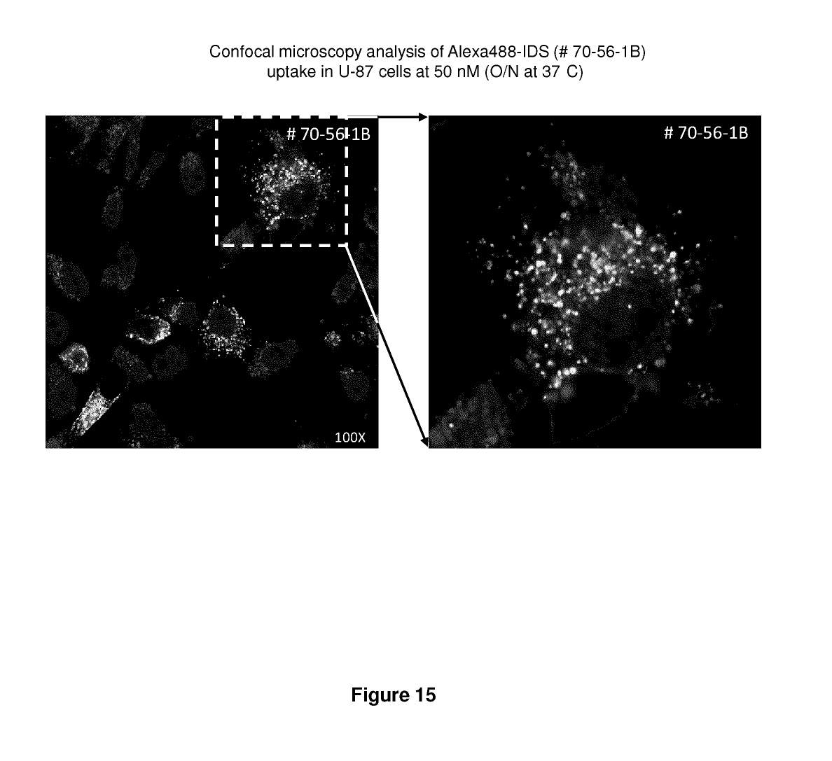

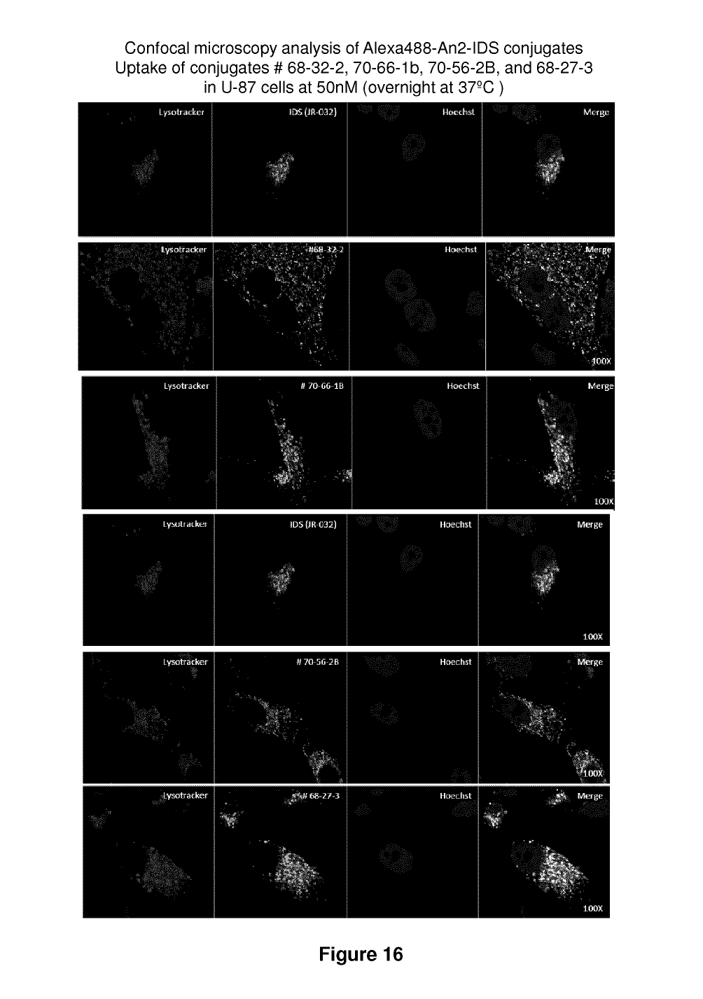

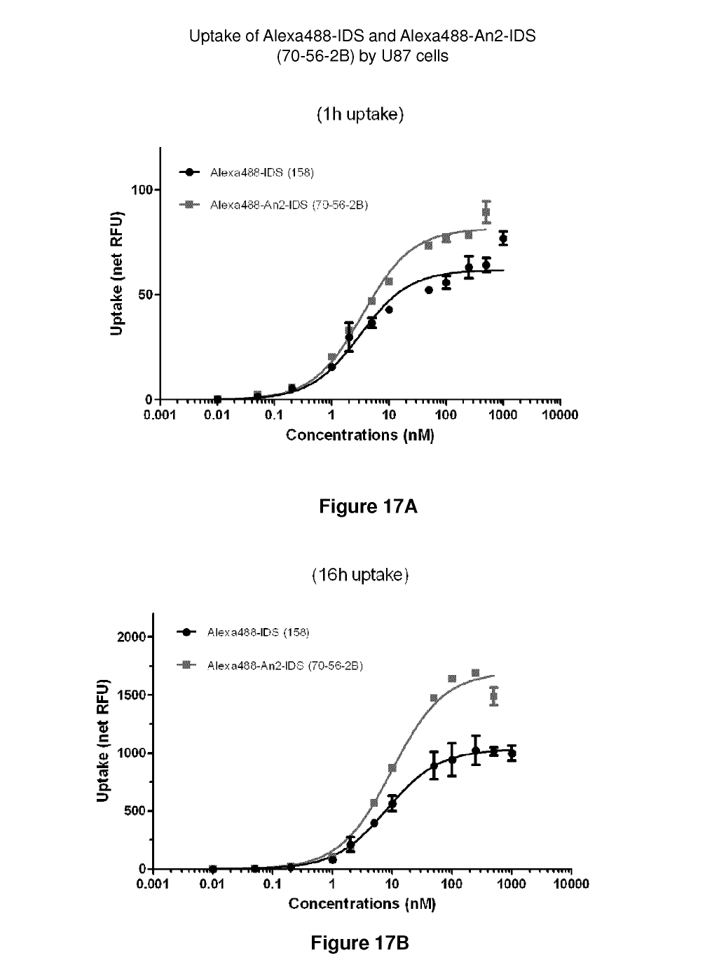

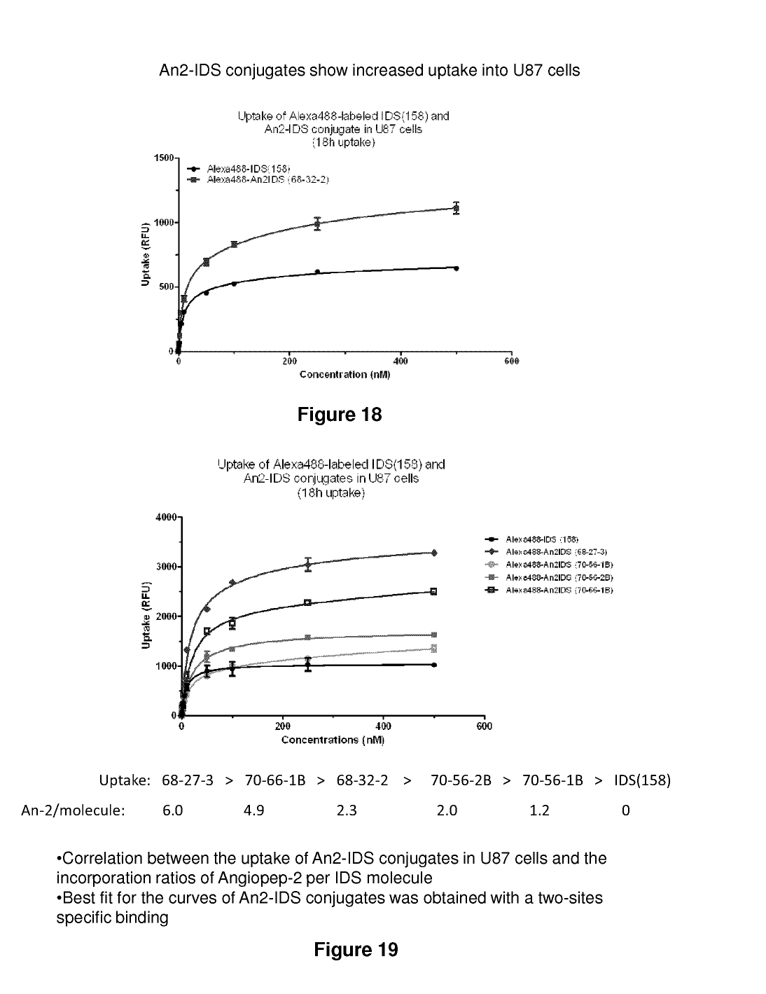

The conjugation products were labeled with Alexa 488 dye and used in trafficking studies in U87 cells to compare their localization with that of the lysotracker dye. A schematic of the microscopy experiment is provided in FIG. 11 and results of the confocal microscopy of 68-32-2, 70-56-1B, 70-56-2B, and 70-66-1B conjugates, labeled with Alexa 488 dye, showing their localization relative to the lysotracker dye are shown in FIGS. 12-16. Colocalization of a conjugate with the lysotracker dye indicated the presence of that conjugate in acidic lysosomes. FIG. 17 shows quantitation of data showing that the entry of both conjugated and native JR-032 was observed following a 1 hour or 16 hour (FIG. 17) incubation. The uptake EC₅₀ is approximately 10 nM for both enzymes, with a higher maximal uptake demonstrated for 70-56-2B. Further data supporting the uptake of An2-IDS into U-87 cells is shown in FIGS. 18 and 19.

### Example 11

### Identification of the Modified Lysines

To determine the lysines in IDS to which An2 was conjugated, JR-032, 70-56-2B, 70-66-1B, and 68-32-2B and their intermediates were subjected to trypsin digestion. The proteolytic fragments of each protein were subsequently analyzed by LCMSMS. From this analysis, eight lysines on JR-032 have been identified as locations that modification occurs during the synthesis of An2-IDS conjugates as shown in Table 4.

TABLE 4
Summary of identified modified lysines on An2-IDS
conjugates/intermediates

| An2 IDS | | Identified | Number of |
|---|---|---|---|
| Conjugates/ | | Modified IDS | modified |
| Intermediate | Coverage | Lysine | lysines |
| JR-032 | 66-87% | None | |
| 70-56-2B | | | 5 |
| Intermediate | 80% | K199, K479, | |
| | | K483 | |
| Conjugate | 74% | K199, K376 | |
| 70-66-1B | | | 3 |
| Intermediate | 59% | K479 | |
| Conjugate | 67% | K199, K211, | |
| | | K376 | |
| 68-32-2B | | | 8 |
| Intermediate | 78% | K199, K240, | |
| | | K295, K347, | |
| | | K479, K483, | |
| Conjugate | 72% | K199, K479 | |

### Example 12

### IDS Enzymatic Specific Activity of Conjugates

### Protocol:

1) Determine the concentration of proteins in JR-032 and conjugates by microBCA.

### 2) Preparation of the Test Solution:

Dilute JR-032 and conjugates 1/200 in Triton-X100 containing diluted buffer.

3) Prepare Standard Solution by diluting 1 mL 4-MU Stock Solution ((4-methylumbelliferone; 0.01 mol/L) in 11.5 mL of Triton-X100 containing buffer (final concentration 800 μmol/L).
4) Prepare serial dilutions of Standard Solution by diluting 500 μL of 800 μmol/L in 500 μL of Triton X100 containing buffer to make a 400 μmol/L solution. Repeat the process to have the following dilutions: 800, 400, 200, 100, 50, 25, 12.5 and 6.25 μmol/L.
5) Distribute 10 μL each of the blank solution (Triton-X100 containing diluted buffer) in 2 wells (n=2), standard solutions (6.25 μmol/L to 800 μmol/L) in 2 wells (n=2) and the test solutions in 4 wells each (n=4) of a microplate, respectively.
6) To each well, add 100 μL of the substrate solution (4-methylumbelliferyl sulfate potassium salt; 4-MUS in 5 mM acetate buffer (pH 4.5) containing 0.1% (w/v) Triton-X-100 and 0.05% (w/v) BSA) and mix gently.
7) Cover the plate and place in an incubator adjusted to 37° C.
8) Add 190 μL of the stop solution (0.33 M glycine and 0.21 M sodium carbonate pH 10.7) to each well exactly after 60 minutes and mix to stop the reaction.
9) Set the plate in the fluorescence plate reader and determine fluorescence intensity at excitation wavelength of 355 nm and detection wavelength of 460 nm.
10) Perform the same measurement with the reference material if comparison is required among tests.

### Method of Calculation:

11) Determine the concentration of 4-MU, Cu(μmol/L), in the test wells using the following formula.

$$Cu = Cs ( \frac{Au}{As} )$$

Cu: Concentration (μmol/L) in the test solution
Cs: Concentration (μmol/L) in the standard solution
Au: Fluorescence intensity of the test solution
As: Fluorescence intensity of the standard solution
12) Specific activity of the test solution: Determine the specific activity, B (mU/mg), of the test solution using the following formula:

$$B = \frac{\frac{Cu}{60} \times C \times \frac{50}{0.1}}{P}$$

C: Dilution factor of the desalted test substance
B: Specific activity (mU/mg)
P: Concentration (mg/mL) of proteins in the desalted test substance

### Results

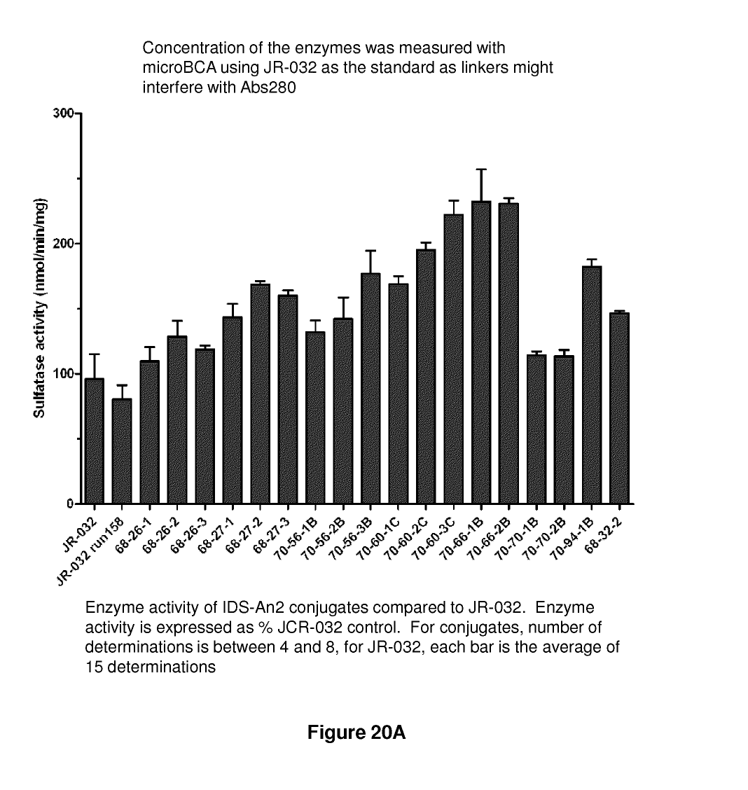

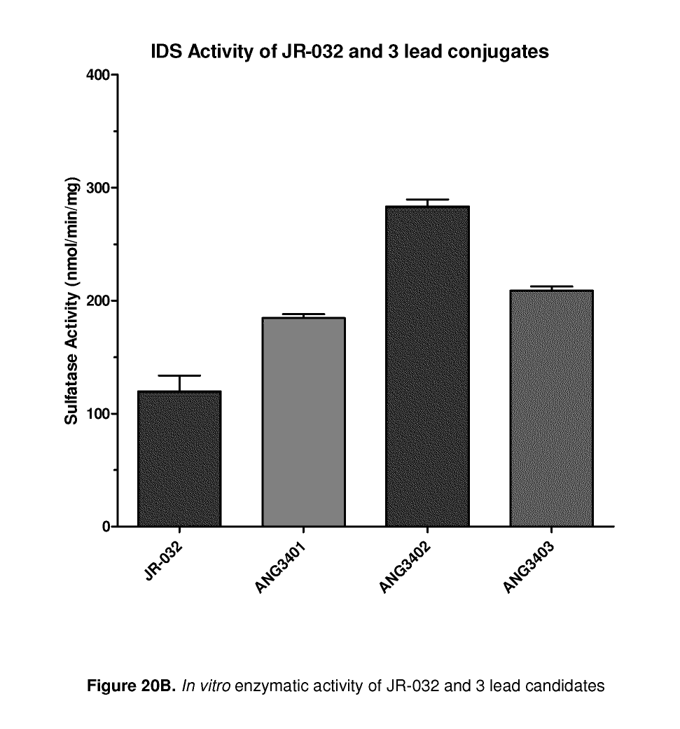

Lysine conjugates were subjected to in vitro enzyme assays with JR-032 as a control. All conjugates retain enzyme activity (see FIG. 20A). In some cases, measured activity exceeds that of native IDS. This may result from interference in the protein quantification assay, leading to a lower calculated protein concentration and higher activity/protein or could indicate that modification of the enzyme positively modulates its activity. FIG. 20B shows results of a further comparison of in vitro enzymatic activity of JR032 (passed over a Q-Sepharose column to remove Tween-80) and large scale syntheses of 70-56-2B, 70-66-1B and 68-32-2.

### Example 13

### Glycosaminoglycan (GAG) Accumulation Assay

### Protocol:

### Materials:

Type II MPS Hunter fibroblasts (Coriell institute, GM00298)
Healthy human fibroblasts (Coriell institute, GM05659)
Dulbecco's Modification of Eagle's Medium (DMEM), fetal bovine serum (FBS)
low sulfate Ham's F-12 medium (Invitrogen, catalog #11765-054)
FBS dialysed against 0.15 M NaCl, 10000 Da MWCO (Sigma, catalog # F0392)
³⁵S-sodium sulfate (Perkin-Elmer, catalog # NEX041H002MC)

### Method:

1. MPS II (GM00298) or healthy human fibroblasts (GM05659) in
6-well dishes at 250,000 cells/well in DMEM with 10% fetal bovine serum (FBS).
Grow for 4 days.
   2.—Discard medium, wash cells with warm and sterile PBS.
Add 1 mL/well of low sulfate F-12 medium with 10% dialysed FBS and 10 μCi ³⁵S-sodium sulfate.
Add JR-032 or conjugates. Incubate at 37° C., 5% CO₂ for 48 h
   3.—Discard medium, wash cells with cold PBS (1 mL, 5 washes).
Lyse cells in 0.4 mL/well of 1 N NaOH.
Heat at 60° C. for 60 min to solubilize proteins.
Remove and aliquot for μBCA protein assay.
   4. Count radioactivity with a liquid scintillation counter.
   5. microBCA protein assay.
   The data are expressed as ³⁵S CPM per μg protein.

### Results

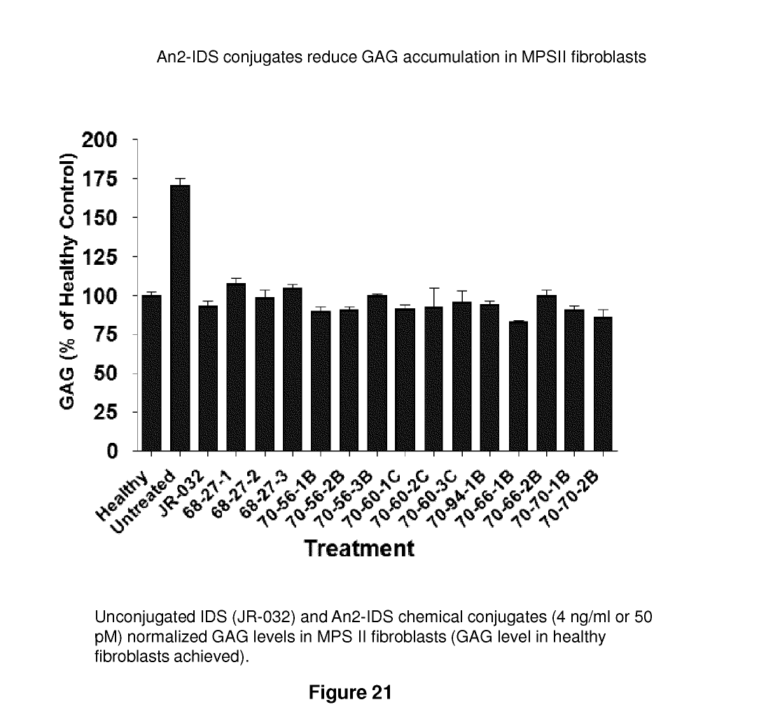

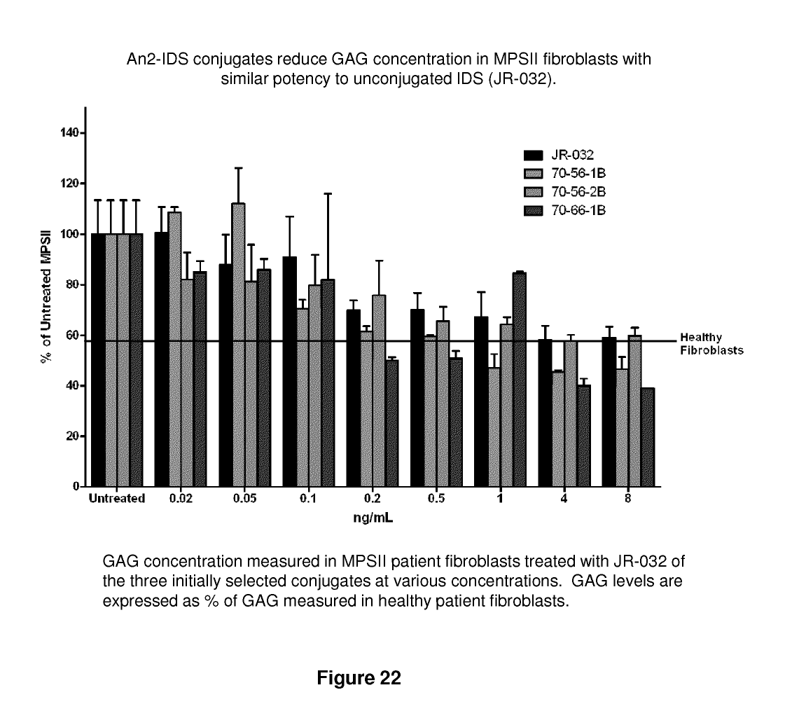

To confirm enzymatic activity with a functional endpoint, the conjugates were assayed for efficacy at reducing GAG levels in fibroblasts from MPSII patients. At a concentration of 4 ng/ml (50 pM), GAG levels are reduced to levels observed in non-disease fibroblasts, similar to that observed with JR-032 (see FIGS. 21 and 22).

### Example 14

### In Situ Brain Perfusion

### Protocol:

JR-032 passed over a Q-sepharose column and conjugates were radiolabeled by standard procedures using an iodo-bead kit and D-Salt Dextran desalting columns from Pierce (Rockford, Ill.). Two iodo-beads were used for the iodination of protein. Briefly, beads were washed twice with 3 ml of PBS on a Whatman filter and resuspended in 60 μl of PBS. Na¹²⁵I (1 mCi) from Amersham-Pharmacia Biotech (Baie d'Urfé, Que) was added to the bead suspension for 5 min at room temperature. Iodination of JR-032 or conjugate was initiated by the addition of 2 mg of protein diluted in 0.1M phosphate buffer solution, pH 6.5. After incubation for 10 min at room temperature, iodo-beads were removed and the supernatants were applied onto a desalting column prepacked with 5 ml of cross-linked dextran from Pierce (Rockford, Ill.). 125I proteins were eluted with 10 ml of PBS. Fractions of 0.5 ml were collected and the radioactivity in 5 μl of each fraction was measured using a Gamma-counter (Perkin-Elmer, 2470 automatic gamma-counter). Fractions corresponding to ¹²⁵I-proteins were pooled and dialyzed (cut-off 10 kDa) against Ringer/Hepes, pH 7.4. Free iodine was removed on a gel filtration column followed by extensive dialysis. Following radiolabeling, SEC analysis demonstrates that over 90% of the radioactivity is associated with the major peak that corresponds to the enzyme fraction. Radiolabeled proteins were dosed using the Bradford assay with JR-032 as the standard.

The in situ mice brain perfusion method was established in the laboratory from the protocol described by Dagenais et al., 2000. Briefly, the surgery was performed on sedated mice, injected intraperitoneal (i.p.) with Ketamine/Xylazine (140/8 mg/kg). The right common carotid artery was exposed and ligated at the level of the bifurcation. The common carotid was then catheterized rostrally with polyethylene tubing (0.30 mm i.d.×0.70 mm o.d.) filled with saline/heparin (25 U/ml) solution mounted on a 26-gauge needle.

Prior to surgery, perfusion buffer consisting of KREBS-bicarbonate buffer −9 mM glucose was prepared and incubated at 37° C., pH at 7.4 stabilized with 95% O₂: 5% CO₂. A syringe containing radiolabeled compound added to the perfusion buffer was placed on an infusion pump (Harvard pump PHD2000; Harvard apparatus) and connected to the catheter Immediately before the perfusion, the heart was severed and the brain was perfused for 2 min at a flow rate of 2.5 ml/min. All perfusions for JR-032 and conjugates were performed at a concentration of 5 nM. After perfusion, the brain was briefly perfused with tracer-free solution to wash out the blood vessels for 30 s. At the end of the perfusion, the mice were immediately sacrificed by decapitation and the right hemisphere wass isolated on ice and homogenized in Ringer/Hepes buffer before being subjected to capillary depletion.

### Capillary Depletion

The capillary depletion method allows the measure of the accumulation of the perfused molecule into the brain parenchyma by eliminating the binding of tracer to capillaries. The capillary depletion protocol was adapted from the method described by Triguero et al., 1990. A solution of Dextran (35%) was added to the brain homogenate to give a final concentration of 17.5%. After thorough mixing by hand the mixture was centrifuged (10 minutes at 10000 rpm). The resulting pellet contains mainly the capillaries and the supernatant corresponds to the brain parenchyma.

### Determination of Tracer Signal

Aliquots of homogenates, supernatants, pellets and perfusates were taken to measure their contents in radiolabeled molecules. [¹²⁵I]-samples were counted in a Wizard 1470 Automatic Gamma Counter (Perkin-Elmer Inc, Woodbridge, ON). All aliquots were precipitated with trichloroacetic acid (TCA) in order to get the radiolabeled precipitated protein fractions. Results are expressed in term of volume distribution (ml/100 g/2 min) for the different brain compartments.

### Results

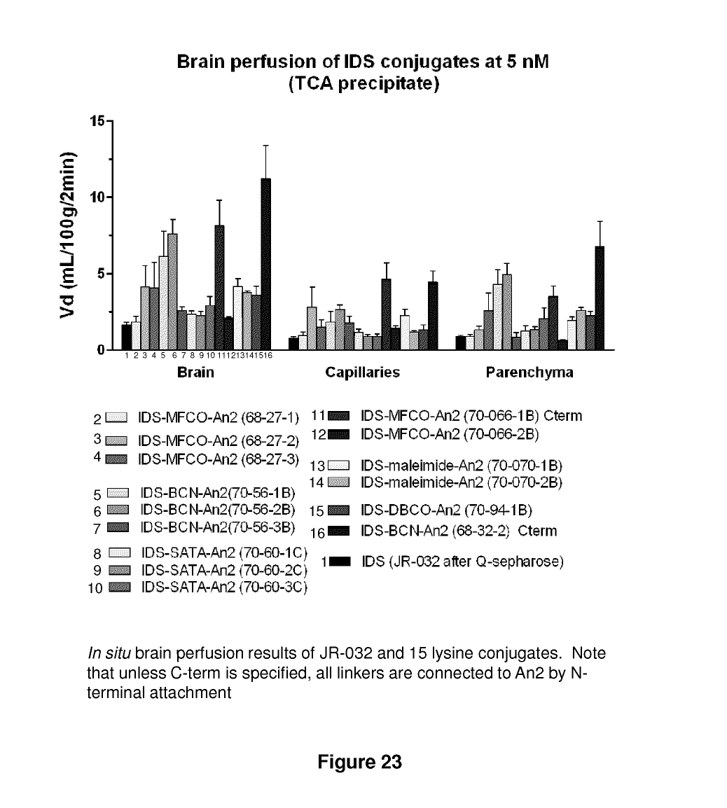

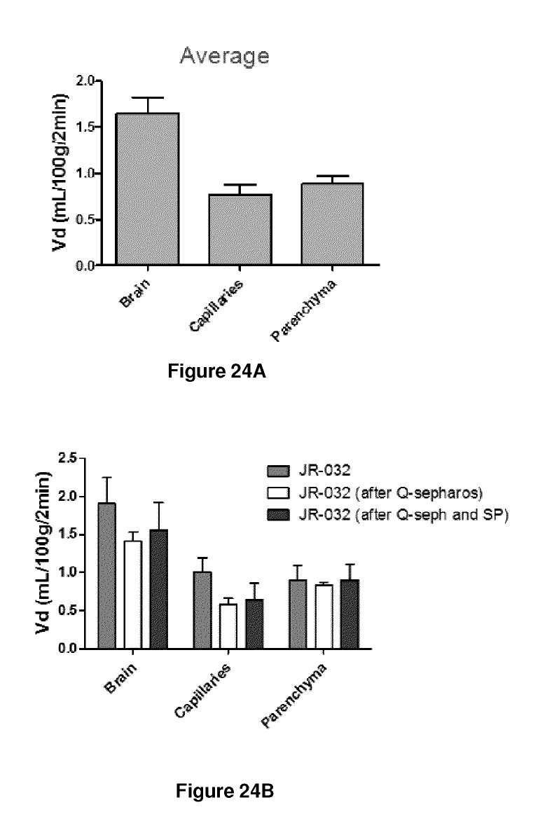

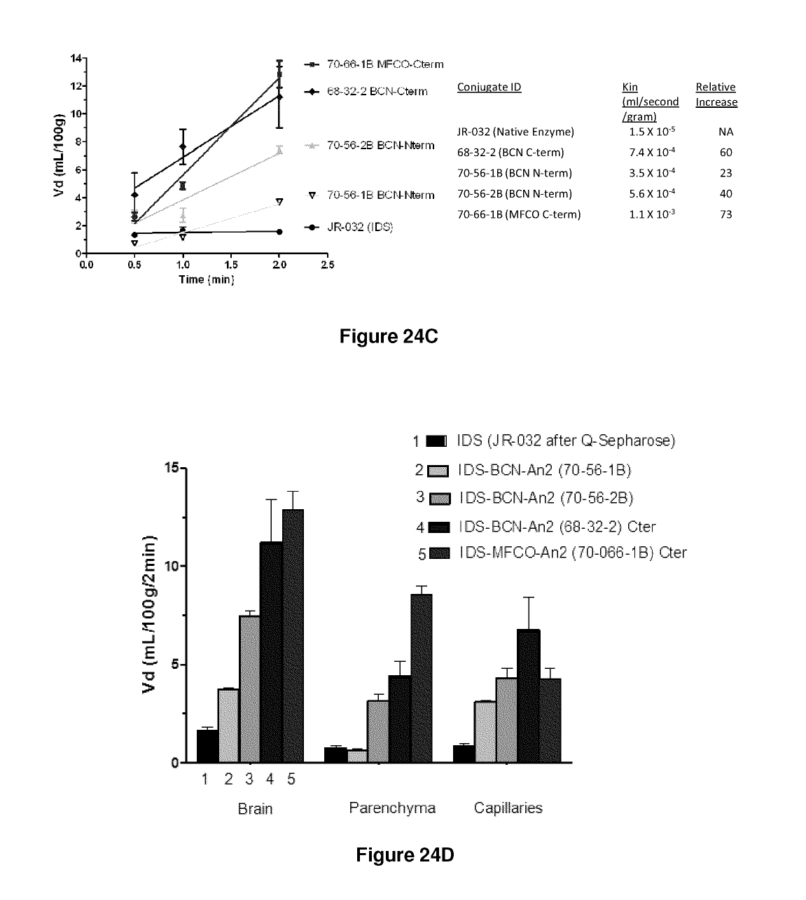

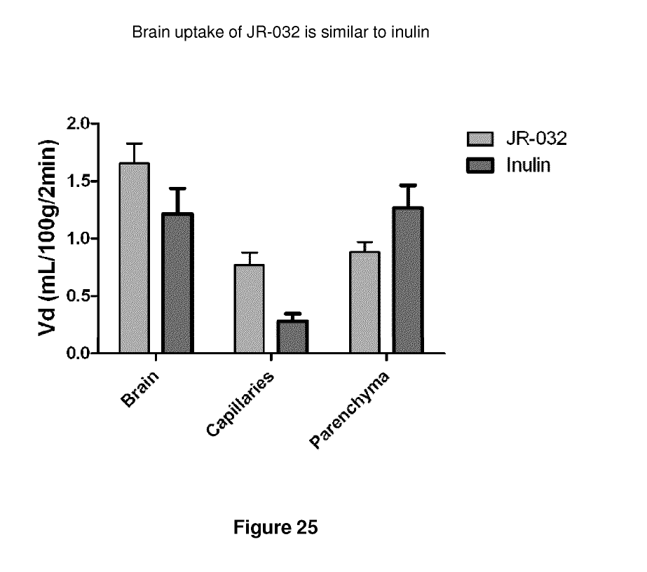

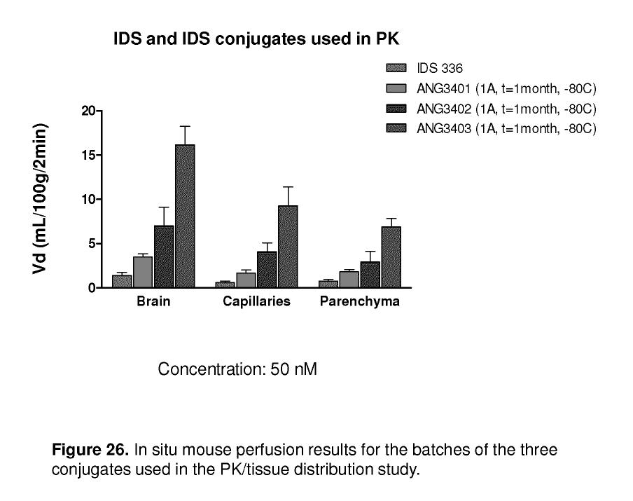

To determine whether conjugation confers an advantage with respect to brain penetration, conjugates were radio-iodinated and tested in the in situ brain perfusion assay in mouse. In this experiment, enzyme (5 nM) is delivered via the carotid artery, thereby maximizing the amount delivered selectively to brain. Following a two minute exposure, the brain was perfused with saline to remove circulating enzyme. Upon removal of the brain, a capillary depletion protocol was used to separate capillary-associated and parenchymal fractions. Radioactivity was counted to quantify the volume of distribution of the test article. JR-032 was used as a control in all experiments and its results were pooled to generate a single control value. FIGS. 23 and 24 show the brain distribution of JR-032 and conjugates respectively at a single time point (2 minutes). A comparison of the brain distribution of JR-032 relative to inulin is provided in FIG. 25. FIG. 26 provides the results of a further 2 minute in vivo brain permeability study conducted in mice of iodinated JR032 (passed over a Q-Sepharose column to remove Tween-80) and large scale syntheses of 70-56-2B, 70-66-1B and 68-32-2 in total brain, capillary and parenchyma. The parenchyma, composed of neurons and glial cells that lie inside of the BBB, represents the targeted area for therapeutic efficacy of the enzyme. For 70-56-2B, 70-66-1B and 68-32-2, exposure in all compartments is higher than that of native enzyme.

### Example 15

### PK/Tissue Distribution in Wild Type Mice

### Protocol:

C57B16/J (Jackson Laboratories) mice age 12 weeks were dosed with a single iv tail-vein administration of ¹²⁵I enzyme at either 1 mg/kg or 5 mg/kg. Tissue and plasma were collected according to the schedule in Table 5 (P: plasma, T: tissue). Blood was collected in EDTA coated microtubes (microcuvette capillary Di-Kalium EDTA). Plasma samples were obtained after centrifugation at 5000 rpm for 10 minutes (Beckman Coulter Microfuge 22R Centrifuge). Prior to sacrifice for tissue collection, mice were perfused by the heart left ventricle with ice-cold 40 ml saline (5 ml/min, 8 minutes). Tissues collected were brain, liver, heart, lung, skin, muscle, spleen, kidney, bone and cartilage. Tissues were collected and weighed (Balance Denver Instrument S-403) in preweighed tubes (Sarstedt 12×75 mm round base). Radioactivity levels in blood plasma (10 μL) and tissues were counted by gamma counting on a Wizard 1470 Automatic Gamma Counter (Perkin-Elmer Inc, Woodbridge, ON).

TABLE 5
Plasma and tissue collection schedule

| 3 point | | | | | | | | | Total | Total |
|---|---|---|---|---|---|---|---|---|---|---|
| composite | 0.25 | 0.5 | 1 | 4 | 6 | 8 | 24 | 48 | mice* | Drug* |
| Group A | P | P | P & | | | | | | | |
| | | | T | | | | | | | |
| Group B | | | | P | P | P& | | | | |
| | | | | | | T | | | | |
| Group C | | | | | | | P | P& | 18 | 2.2 mg |
| | | | | | | | | T | | |

Mathematical analysis of data was performed using Excel spreadsheets. Raw data was collected as cpm/mg tissue and converted to ng/g. For plasma samples, radioactivity counts were converted to μg/ml of plasma. Pharmacokinetic analysis was performed using WinNonlin_Professional version 5.2 (Pharsight Corporation, Mountain View, Calif.).

Data are expressed as mean±S.E.M. All statistical analyses were performed using GraphPad Prism version 5.0 (GraphPad Software Inc., San Diego, Calif.).

### Results

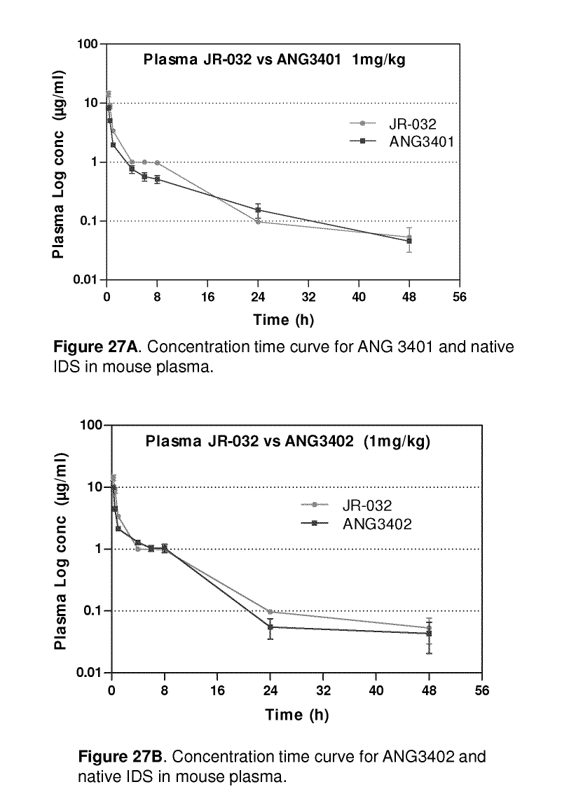

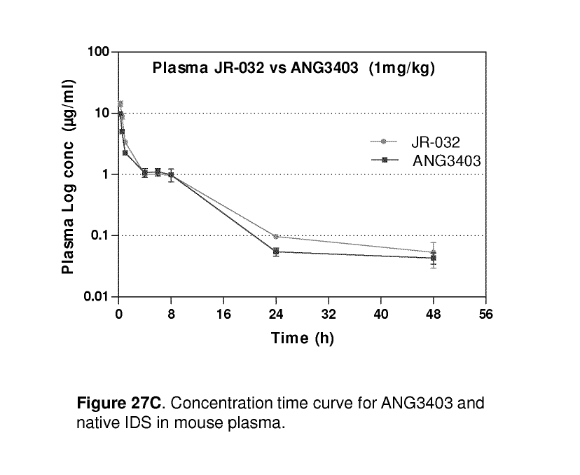

For PK analysis, amount of enzyme in plasma was determined by radioactive counting. Plasma concentration vs. time curves are shown in FIG. 27, with values for analysed parameters in Table 6. For all enzymes, Tmax is observed at 15 minutes. As expected for the conjugates, plasma AUC, and Cmax are lower than for JR-032, while clearance and volume of distribution are higher, consistent with the possibility that the conjugates partition from plasma to tissue more rapidly. The AUC_{%}, or the percent of the AUC_{∞} that is based on extrapolation, is low for all enzymes, suggesting that greater than 95% of the overall plasma exposure is represented by the AUC₀₋₄₈.

TABLE 6
Plasma PK parameters for JR-032 and conjugates

| | JR-032 | 70-56-2B | 70-66-1B | 68-32-2 |
|---|---|---|---|---|
| Cmax (μg/ml) | 14.4 | 8.3 | 10.0 | 9.7 |
| Tmax (hr) | 0.25 | 0.25 | 0.25 | 0.25 |
| AUC₀₋₄₈ (hr * μg/ml) | 31.6 | 20.4 | 27.0 | 25.9 |
| AUC_{0-∞} (hr * μg/ml) | 32.3 | 21.2 | 27.5 | 26.5 |
| AUC_{%} | 2.4 | 4.1 | 1.9 | 2.0 |
| T½β (hr) | 10.1 | 12.1 | 9.1 | 9.3 |
| k (1/hr) | 0.069 | 0.057 | 0.076 | 0.075 |
| Cl (ml/hr) | 0.82 | 1.3 | 1.1 | 1.1 |
| Vd (ml/kg) | 443 | 825 | 539 | 544 |

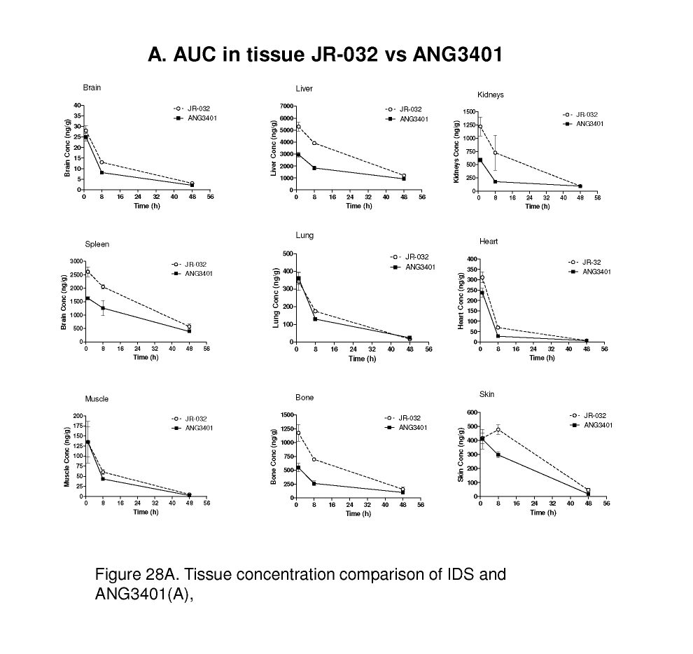

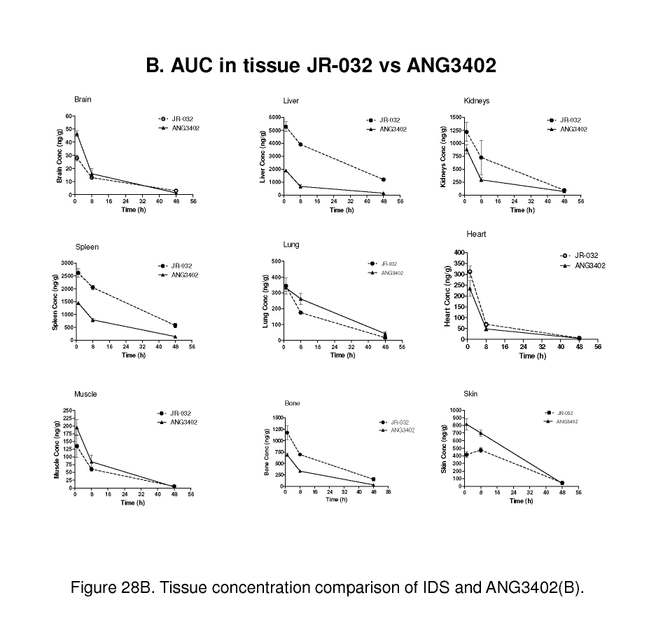

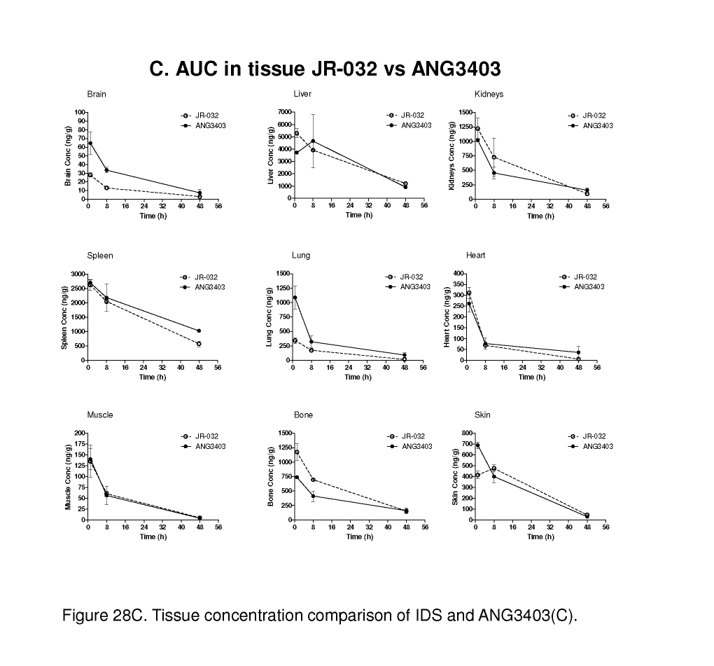

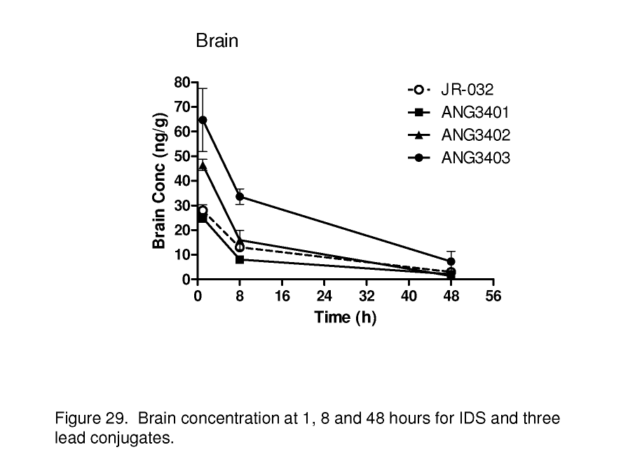

Concentrations of conjugates compared with IDS in tissues at 1 hour, 8 hours and 48 hours are shown in FIG. 28, with a comparison of all enzyme concentrations in brain shown in FIG. 29. For 70-56-2B (FIG. 28A), concentrations are similar to or lower than those of native IDS for all tissues at all time-points tested. For 70-66-1B (FIG. 28B), concentrations are similar to concentrations of IDS in heart, higher in brain, lung, muscle and skin, and lower in liver, kidney, spleen and bone. For 68-32-2 (FIG. 28C), concentrations are higher than IDS in brain and lung, similar in heart, spleen, muscle and skin, and slightly lower in liver, bone and kidney. As can be seen in FIG. 29, the rank order of brain concentration is 68-32-2 (highest), followed by 70-66-1B and 70-56-2B. This rank order is consistent with that observed in the mouse brain permeability study shown in FIG. 26, suggesting that the in situ brain perfusion technique is highly predictive of brain exposure. AUC values for JR-032 and conjugates, and ratios (conjugate:JR-032) can be found in Table 7.

TABLE 7
AUC values for JR-032 and conjugates, in ng/g*hr. Fold over JR-032AUC is shown in parenthesis.

| | AUC | Brain | Heart | Liver | Lungs | Kidney | Muscle | Skin | Bone | Spleen |
|---|---|---|---|---|---|---|---|---|---|---|
| JR-032 | 1 h-8 h | 143.7 | 1332.6 | 32196.6 | 1809.4 | 6823.0 | 684.6 | 3228.8 | 6545.9 | 16341.1 |
| | 1 h-48 h | 465.1 | 2836.9 | 174255.4 | 5616.1 | 23264.4 | 1995.0 | 14295.4 | 23533.8 | 68890.6 |
| | 0-∞ | 563.1 | 3267.4 | 539907.6 | 6231.4 | 26318.1 | 2213.0 | 15609.4 | 28475.0 | 89432.4 |
| 68-32-2 | 1 h-8 h | 344.3 | 1187.3 | 29314.9 | 4942.0 | 5180.7 | 688.6 | 3808.2 | 4031.3 | 17083.6 |
| | | (2.4) | (0.9) | (0.9) | (2.7) | (0.8) | (1.0) | (1.2) | (0.6) | (1.0) |
| | 1 h-48 h | 1163.4 | 3472.5 | 140738.8 | 13268.8 | 17436.4 | 1899.2 | 12380.2 | 15521.3 | 81268.4 |
| | | (2.5) | (1.2) | (0.8) | (2.4) | (0.7) | (1.0) | (0.9) | (0.7) | (1.2) |
| | 0-∞ | 1399.1 | 4971.8 | 171491.0 | 16038.2 | 22797.1 | 2057.3 | 13261.8 | 21805.6 | 138131.5 |
| | | (2.5) | (1.5) | (0.3) | (2.6) | (0.9) | (0.9) | (0.8) | (0.8) | (1.5) |
| 70-66-1B | 1 h-8 h | 218.6 | 989.9 | 9031.8 | 2076.8 | 4133.8 | 979.7 | 5306.4 | 3576.5 | 7868.2 |
| | | (1.5) | (0.7) | (0.3) | (1.1) | (0.6) | (1.4) | (1.6) | (0.5) | (0.5) |
| | 1 h-48 h | 565.2 | 2061.0 | 25533.9 | 8140.1 | 11456.8 | 2738.0 | 20074.5 | 10844.1 | 26624.7 |
| | | (1.2) | (0.7) | (0.1) | (1.4) | (0.5) | (1.4) | (1.4) | (0.5) | (0.4) |
| | 0-∞ | 634.6 | 2391.1 | 30636.1 | 9405.0 | 13859.7 | 2986.2 | 21478.5 | 12071.0 | 31229.4 |
| | | (1.1) | (0.7) | (0.1) | (1.5) | (0.5) | (1.3) | (1.4) | (0.4) | (0.3) |
| 70-56-2B | 1 h-8 h | 114.6 | 922.6 | 16408.7 | 2108.0 | 2655.9 | 616.9 | 2418.1 | 2792.3 | 9809.7 |
| | | (0.8) | (0.7) | (0.5) | (1.2) | (0.4) | (0.9) | (0.7) | (0.4) | (0.6) |
| | 1 h-48 h | 310.7 | 1549.9 | 69697.3 | 7396.9 | 7983.6 | 1498.9 | 8424.6 | 9750.2 | 41239.3 |
| | | (0.7) | (0.5) | (0.4) | (1.3) | (0.3) | (0.8) | (0.6) | (0.4) | (0.6) |
| | 0-∞ | 391.4 | 1959.4 | 116934.7 | 5611.3 | 11940.0 | 1720.7 | 9446.6 | 13541.6 | 58133.0 |
| | | (0.7) | (0.6) | (0.2) | (0.9) | (0.5) | (0.8) | (0.6) | (0.5) | (0.7) |

The plasma AUC values for the three conjugates are slightly lower than that of JR-032, which is an expected result given that distribution from plasma to tissue is believed to be accelerated by LRP-1 receptor mediated transcytosis.

One conjugate, 70-56-2B, exhibited a much higher volume of distribution compared to the other conjugates tested; 22 vs. 12-15 ml. This result suggests that conjugation does have an effect on the pharmacokinetics of the enzyme. However, the tissue compartment(s) responsible for this value are unknown as there is no evidence that the nine tissues examined in our study received high exposure to 70-56-2B.

The other conjugates, 70-66-1B and 68-32-2, were shown to exhibit higher brain levels after a single iv administration than levels attained for native JR-032. More particularly, 70-66-1B achieved a higher level of exposure in brain than did native JR-032 at early time points. The highest brain exposure was observed for 68-32-2, with an AUCO-∞ that is 2.5-fold that of native JR-032. The fact that the plasma levels are not increased compared to native enzyme is notable, since achievement of higher brain levels has clearly not been achieved in this case by enhancing plasma stability, thereby increasing the amount of time that the blood supply to the brain contains drug.

### Example 16

### Tissue Distribution in Male Wild Type and Male Hemizygous Iduronate-2-Sulfatase Knock Out Mice

### Protocol

An additional tissue distribution study was conducted as summarized in Table 8. In each phase, the mice received a single intravenous administration of either [¹²⁵I]JR032, [¹²⁵I]70-66-1B or [¹²⁵I]68-32-2 solutions in phosphate buffered saline at a target dose level of 1 mg/kg by bolus injection into a caudal vein. The radioactive dose received by each animal was calculated from the weight of dose formulation administered and its radioactivity concentration.

TABLE 8
Tissue distribution study details

| | Phase | | | | | | | | |
|---|---|---|---|---|---|---|---|---|---|
| | A | B | | C | D | | E | F | |
| | Test Compound | | | | | | | | |
| | [¹²⁵I]JR032 | | | [¹²⁵I]70-66-lB | | | [¹²⁵I]68-32-2 | | |
| | Dose | | | | | | | | |
| | 1 mg/kg | | | 1 mg/kg | | | 1 mg/kg | | |
| | Strain/Model | | | | | | | | |
| | | IDS | | | IDS | | | IDS | |
| | WT | KO | | WT | KO | | WT | KO | |
| Animal | 1M | 3M | | 7M | 9M | | 13M | 15M | |
| number/sex | 2M | 4M | | 8M | 10M | | 14M | 16M | |
| | | 5M | | | 11M | | | 17M | |
| | | 6M | | | 12M | | | 18M | |
| Kill | 1 h | 0.5 | h | 1 h | 0.5 | h | 1 h | 0.5 | h |
| | 8 h | 1 | h | 8 h | 1 | h | 8 h | 1 | h |
| | | 4 | h | | 4 | h | | 4 | h |
| | | 24 | h | | 24 | h | | 24 | h |

| | | | | | | |
|---|---|---|---|---|---|---|
| Analysis | TD | QWBA | TD | QWBA | TD | QWBA |

WT—Wild Type (C57BL/6J)
IDS KO—Iduronate-2-Sulphatase Knock Out
TD—Tissue Distribution (liquid scintillation techniques)
QWBA—Quantitative Whole-Body Autoradiography

### Quantitative Tissue Distribution (Phases A, C and E)

Individual mice were exsanguinated (cardiac puncture under isoflurane/oxygen anaesthesia) and killed (cervical dislocation) at the times listed in the table. After sacrifice, a number of tissues/organs were removed or sampled (as appropriate) from each carcass including: brain, heart, liver, lungs, kidney (cortex), muscle (leg abductor), skin, bone (femur including marrow) and spleen. Aliquots of plasma (ca. 0.05 g) were also taken for measurement of total radioactivity concentrations. The weight and radioactivity concentration of each sample was measured.

Radioactivity concentration was measured using a COBRA II gamma scintillation counter (Model 5003) with the mode of counting pre-set at 4 minutes. All counts were back-calculated by the gamma counter computer software using a half life of 60 days and the reference date of 3 Apr. 2013 for [¹²⁵I]JR032, 11 Apr. 2013 for [¹²⁵I]70-66-1B and 15 Apr. 2013 for [¹²⁵I]68-32-2. A blank ‘background’ disintegration rate was measured with every batch of study samples and subtracted from each sample disintegration rate. Radioactivity in amounts less than twice that of the background concentration in the sample under investigation was considered to be below the limit of accurate quantification (BLQ).

### Results

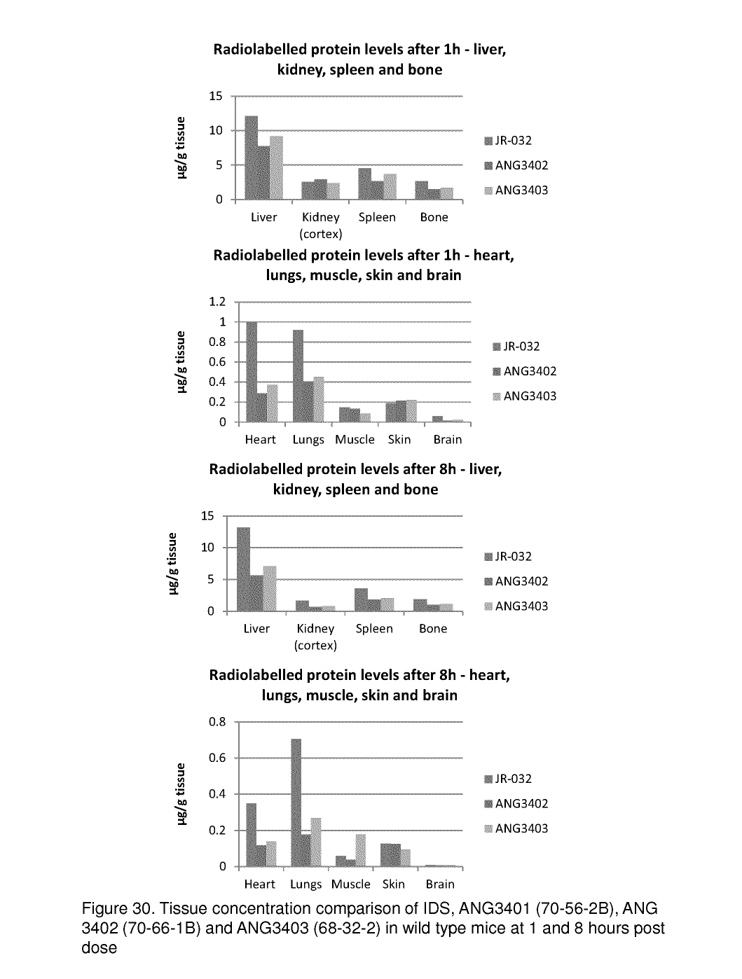

Concentrations of conjugates compared with JR-032 in brain, heart, liver, lungs, kidney (cortex), muscle (leg abductor), skin, bone (femur including marrow) and spleen at 1 hour and 8 hour post dose are shown in FIG. 30. For 70-66-1B at 1 hour, concentrations were lower than IDS in the heart, liver lungs spleen and bone, similar in brain, muscle and skin and slightly higher in kidney cortex. For 68-32-2 at 1 hour, concentrations were lower than IDS in heart, liver, lung, spleen and bone and similar in brain, kidney cortex, muscle and skin. Concentrations of JR-032 and conjugates were lower in each tissue at 8 hours (with the exception of JR-032 in liver at 8 hours). The comparative levels of conjugates to JR-032 were however similar (although 70-66-1B was lower than IDS in kidney cortex at 8 hours).

The concentration of the conjugates in plasma at 1 hour was lower than that of JR-032, which is an expected result given that distribution from plasma to tissue is believed to be accelerated by LRP-1 receptor mediated transcytosis. At 8 hours, the concentration of JR-032 and conjugates in plasma was similar.

### Whole-Body Autoradiography (Phases B, D and F)

After administration of a single intravenous dose of [¹²⁵I]JR032 (Phase B), [¹²⁵I]70-66-1B (Phase D) or [¹²⁵I]68-32-2 (Phase F) to hemizygous IDS knockout mice (n=4 animals/Phase), individual mice were anaesthetised using isoflurane and a sample of whole blood collected from the orbital sinus of the right eye at the time points listed in the table.

Each blood sample (ca 0.2 mL) was transferred to a tube containing heparin anticoagulant, centrifuged (ca. 2000×‘g’ for 10 minutes at ca. 4° C.) with the minimum delay and the separated plasma transferred into a plain tube. Blood cells were discarded.

While still under anaesthesia, mice were pinned to a board and killed by freezing in a bath of hexane/solid CO₂ at ca. −80° C. Following removal of the whiskers, legs and tail, each frozen carcass was set in a block of 2% (w/v) aqueous carboxymethyl cellulose at ca. −80° C.

Samples of fortified human blood (obtained by adding radiolabelled solutions of [¹²⁵I]JR032 (Phase B), [¹²⁵I]70-66-1B (Phase D) and [¹²⁵I]68-32-2 to whole blood obtained from healthy human volunteers to produce nominal concentrations of 0.50, 1.0, 2.5, 5, 100, 1000 and 2000 nCi/mL) containing seven different concentrations of radioactivity were placed into holes drilled into the block to be used to construct a calibration line.

The block was mounted onto the stage of a microtome in a cryostat maintained at ca. −20° C. Sagittal sections (nominally 30 μm) were then obtained at 6 levels through the carcass:

Level A: Kidney

Level B: Intra-orbital lacrimal gland

Level C: Harderian gland

Level D: Adrenal gland

Level E: Half brain and thyroid

Level F: Brain and spinal cord

The sections, mounted on sectioning tape were freeze-dried in a freeze-drier at an average temperature of −55° C. using a Heto Power LL3000 freeze drier. One section from each level was exposed to imaging plates (Raytek Scientific Ltd, Sheffield UK) in a copper and lead-lined exposure box for seven days. The imaging places were scanned using a FLA5000 radioluminography system (Raytek Scientific Ltd, Sheffield, UK). The electronic images were analysed using a validated image analysis package (Seescan Densitometry software, version 1.3).

One replicate freeze-dried section at each level, identical to the quantified sections was mounted on acetate sheets and used for visual reference purposes when evaluating the images.

Concentrations of radioactivity were quantitated in a number of tissues including brain, liver and thyroid. For these tissues the maximum area of each tissue within a single autoradiogram was defined for measurement. The upper and lower limits of quantification in this procedure as given in Table 9.

TABLE 9
Upper and lower limits of quantification

| Dose formulation | | |
|---|---|---|
| type used | LLOQ (μg equivalents/g) | ULOQ (μg equivalents/g) |
| [¹²⁵I]JR032 | 0.003 | 10.0 |
| [¹²⁵I]70-66-1B | 0.003 | 11.8 |
| [¹²⁵I]68-32-2 | 0.004 | 13.0 |

### Results

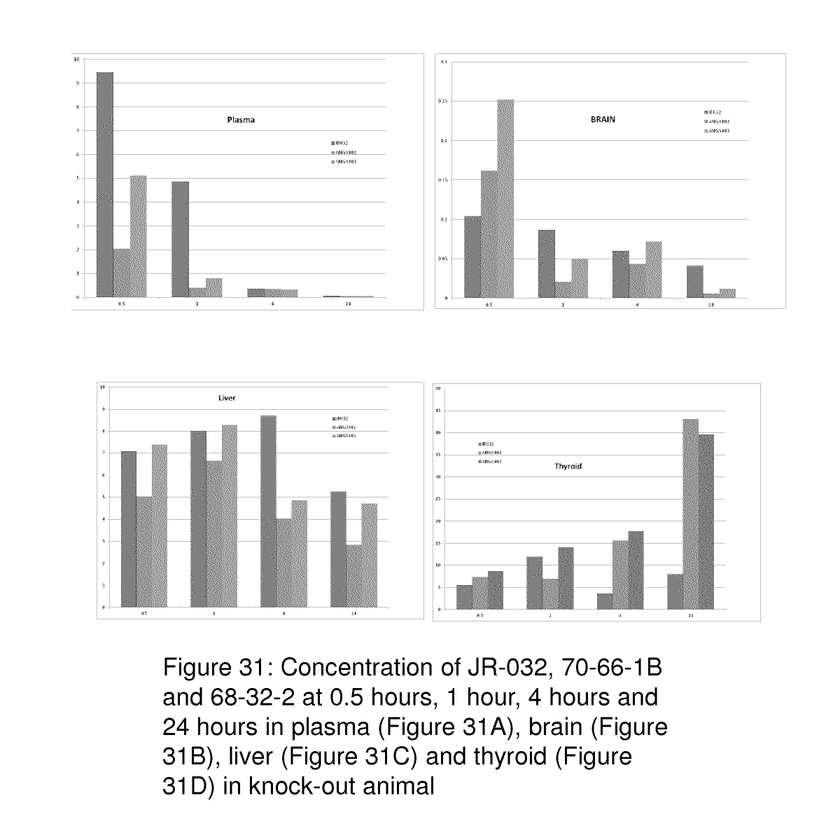

FIG. 31 shows the concentration of JR-032, 70-66-1B and 68-32-2 at 0.5 hours, 1 hour, 4 hours and 24 hours in plasma, brain, liver and thyroid. In brain, the concentrations of both conjugates were higher than JR-032 at 0.5 hours. The concentration of the JR-032 and both conjugates was lower at later time points. In addition, the concentration of both conjugates were lower than JR-032 at 1 hour and 24 hours and similar at 4 hours. Levels of JR-032 and conjugates were much higher in liver and thyroid than in the brain and the comparative levels of conjugates to JR-032 differs significantly from the pattern observed for brain.

Table 10 shows the percentage of radioactivity in the plasma samples that precipitated with 15% aqueous TCA. This shows the percentage of the radioactive iodine isotope that was associated with protein, such as JCR-032 or conjugate.

TABLE 10
Percentage of radioactivity in plasma samples that
precipitated with 15% TCA

| % precipitation | 0.5 h | 1 h | 4 h | 24 h |
|---|---|---|---|---|
| JR032 | 96 | 95 | 71 | 78 |
| 70-66-1B | 83 | 72 | 47 | 73 |
| 68-32-2 | 89 | 75 | 62 | 75 |

### Example 17

### Processing of JR-032 and Conjugates

Iduronate-2-sulfatase (90 kDa) is processed in fibroblasts through various intermediates to the major 55 kDa intermediate, then to the 45 kDa mature form (Froissart et al. Biochem J. 309:425-430, 1995). To determine if the conjugates were processed in a similar manner as the unconjugated enzyme, samples of ANG3402 and ANG3403 were analyzed and compared with samples of JR-032 by Western blot.

### Results

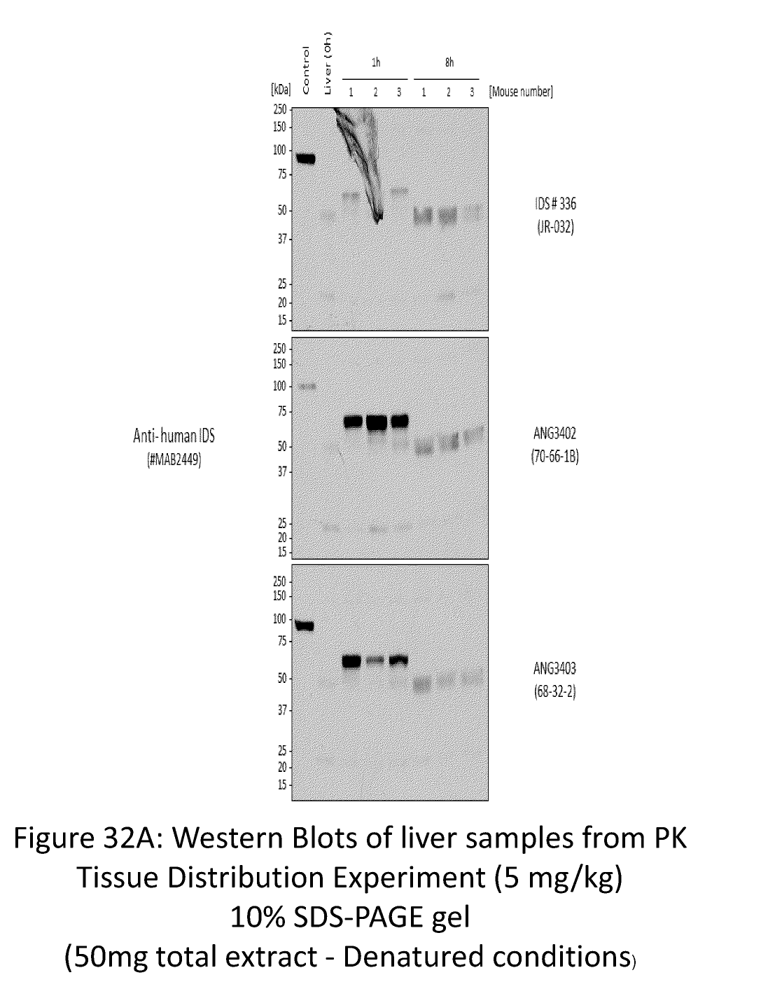

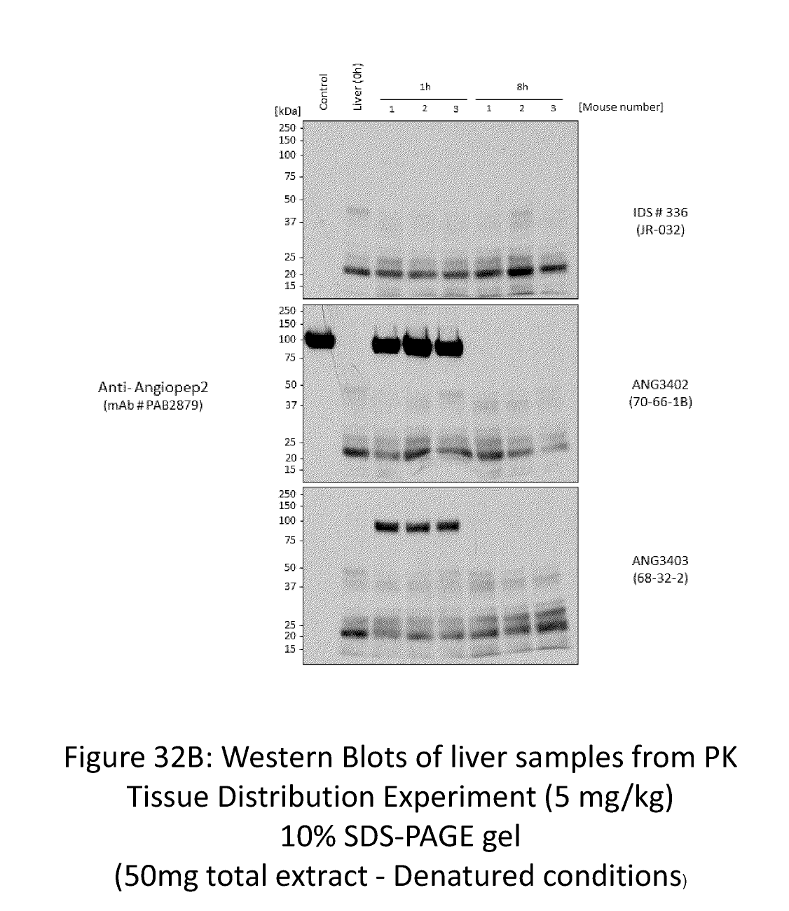

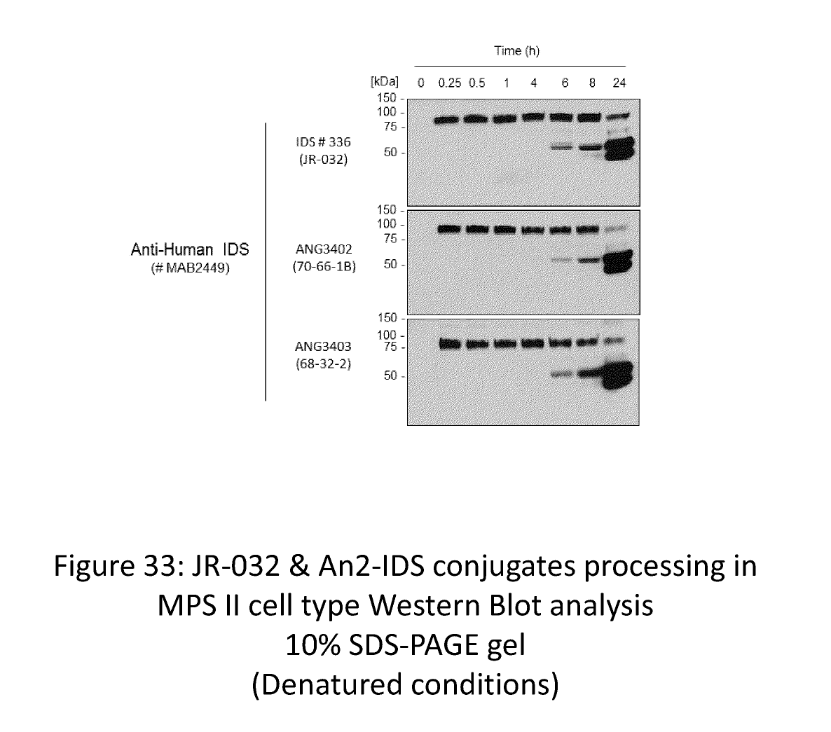

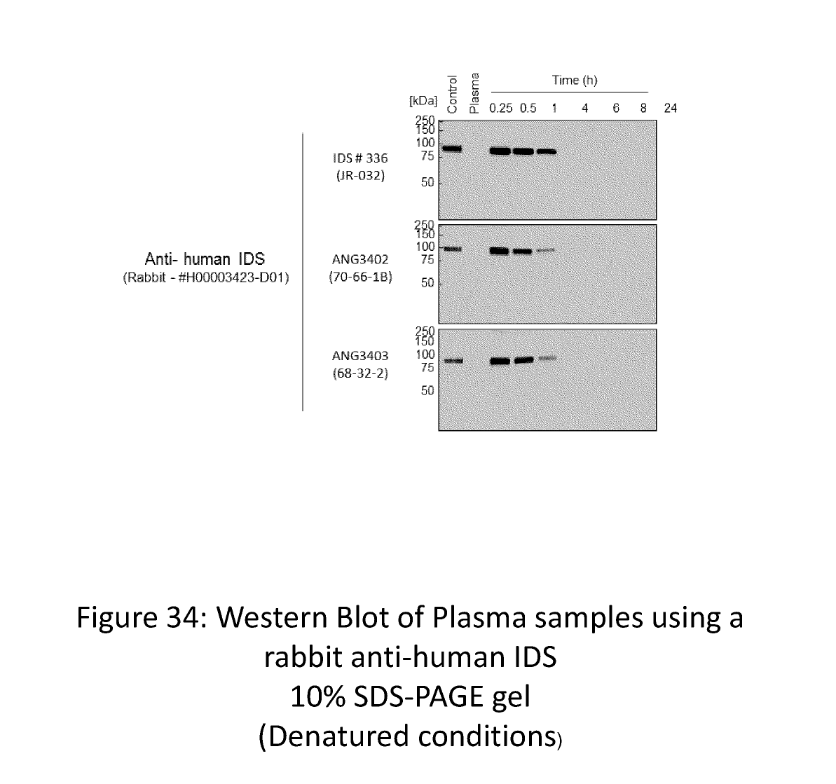

As shown in FIGS. 32A and 32B, ANG3402 and ANG3403 are processed similarly to the unconjugated enzyme. This result was confirmed by comparing samples of ANG3402 and ANG3403 and JR-032 processed in MPS-II fibroblasts (FIG. 33). No apparent processing of JR-032 or the conjugates occurred in the plasma as shown in FIG. 34.

### Example 18

### Effect of Conjugates on GAG Accumulation in the Brain, Heart and Liver in Hemizygous Iduronate-2-Sulfatase Knock Out Mice

### Protocol

Male hemizygous iduronate-2-sulfatase gene knock-out mice (supplied by Oriental BioService Inc.; Minamiyamashiro Laboratory) aged between 21-23 weeks were dosed via injection into the caudal vein once a week for 4 weeks according to Table 11. Male wild type animals (23 weeks old) were used in test group 1 as a control.

TABLE 11
Dosing protocol for GAG accumulation study

| | Type | | Dosing | |
|---|---|---|---|---|
| Test group | of animal | Dosage (mg/kg) | frequency | No of animals |
| 1. Wild type | WT | Not dosed | Not dosed | 5 |
| 2. JR-032 | Hemi | 2 | once/week | 5 |
| 3. Vehicle | Hemi | 2 | once/week | 5 |
| 4. ANG3403 | Hemi | 2 | once/week | 5 |
| 5. ANG3402 | Hemi | 2 | once/week | 5 |

JR-032, 68-32-2 or 70-66-1B were administered in vehicle (8 g/L sodium chloride, 2.65 g/L sodium dihydrogen phosphate, 1.07 g dibasic sodium phosphate hydrate (pH in range 5.86-6.14) filtered using a 0.22 μM sterile syringe). In test group 3, vehicle only was administered.

One week after completion of the 4 week administration, surviving animals were euthanized by bleeding from the abdominal aorta under 20% isoflurane anesthesia and the brain (cerebrum and cerebellum), heart and liver were removed and frozen with liquid nitrogen. The frozen tissues were freeze-dried, cut into small pieces, and weighed. 0.5 mol/L tris HCl buffer solution (pH 7.5) containing 50 mg/mL actinase E was added such that the total additive amount of the solution is 1 ml per 100 mg dry weight of the tissues. The mixture was heated at 100° C. for 10 minutes using a dry bath incubator. Additional 0.5 mol/L tris HCl buffer solution (pH 7.5) containing 50 mg/mL actinase E was then added such that the ratio of the dry weight of the tissues to actinase E is 10 mg to 1 mg and the mixture was incubated at 60° C. for about 16 hours using a dry heat sterilizer. The mixture was then heated at 100° C. for 10 minutes using a dry bath incubator followed by centrifugation at 24° C. at about 20400 g for 10 minutes. The supernatant was removed and frozen for more than 12 hours. The supernatant was then thawed at room temperature and centrifuged again at 24° C. at about 20400 g for 10 minutes. The supernatant was removed and frozen.

A Wieslab® sGAG quantitative kit (EURO-DIAGNOSTICA) was used to determine GAG concentrations twice in 50 μl samples from each tissue, in a 50 μl blank sample (water for injection) and in 50 μl calibration samples (solutions of chondroitin sulfate B sodium salt in water for injection at concentrations of 640 μg/ml, 320 μg/ml, 160 μg/ml, 80 μg/ml 40 μg/ml and 20 μg/ml).

Briefly, 50 μl of 8 M guanidine-HCl was added to each sample and the mixture was allowed to react at room temperature for 15 minutes. 50 μl of SAT solution (0.3% H₂SO₄ and 0.75% Triton X-100) was then added and the mixture was allowed to react at room temperature for 15 minutes. 750 μl of Alcian Blue working solution (prepared by mixing water for injection, the SAT solution and Alcian Blue stock solution (0.1% H₂SO₄ and 0.4 M guandine HCl) at a ratio of 9:5:1) was added to each sample and the mixture was allowed to react at room temperature for 15 minutes. The samples were then centrifuged at 12600 g for 15 minutes at 24° C. and the supernatant was discarded. 500 μl of DMSO solution (40% dimethylsulphoxide and 0.05 M MgCl₂) was added to the tube and the contents of the tube were stirred with a mixer at room temperature for 15 minutes followed by centrifugation at 12600 g for 15 minutes at 24° C. The supernatant was discarded and 500 μl Gu-Prop (4 M guanidine-HCl, 33% 1-propanol and 0.25% Triton X-100) was added to each sample. The tubes were stirred with a mixer at room temperature for 15 minutes so that precipitates were completely dissolved. 200 μl of each sample was dispensed into wells in a 96 well microplate. A microplate reader was used to obtain absorbance values at a wavelength of 600 nm. GAG concentrations were calculated by a linear method with analysis software (KC4 v3.4 DS Pharma Biomedical Co., Ltd.). Mean values (μg/ml) of 2 measurements of GAG concentrations were calculated.

The accuracy of GAG concentrations of the calibration samples were calculated. When the accuracy and correlation coefficient were not within the evaluation criteria, GAG concentrations in the tissue samples were not calculated. (Evaluation criteria for accuracy: coefficient of variation is within 15% (within 20% for the 20 μg/ml sample). Evaluation criteria for correlation coefficient: 0.997 or higher (rounded to 4 decimal places)).

The GAG concentrations measured in the tissue samples were converted to the concentration in the dry weight of each tissue by the following formula:

$$[ GAG ] d = \frac{[ GAG ] s \times A}{W}$$

[GAG]d=GAG concentration in dry tissue (μg/mL)
[GAG]s=GAG concentration in sample (μg/mL)
A=Additive amount (ml)
W=Dry tissue weight (mg)

### Results

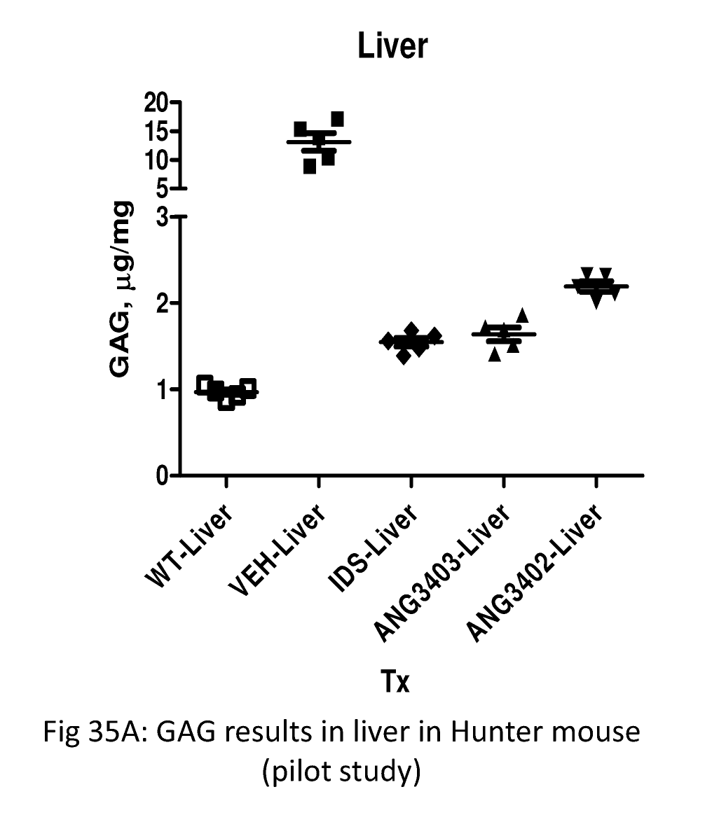

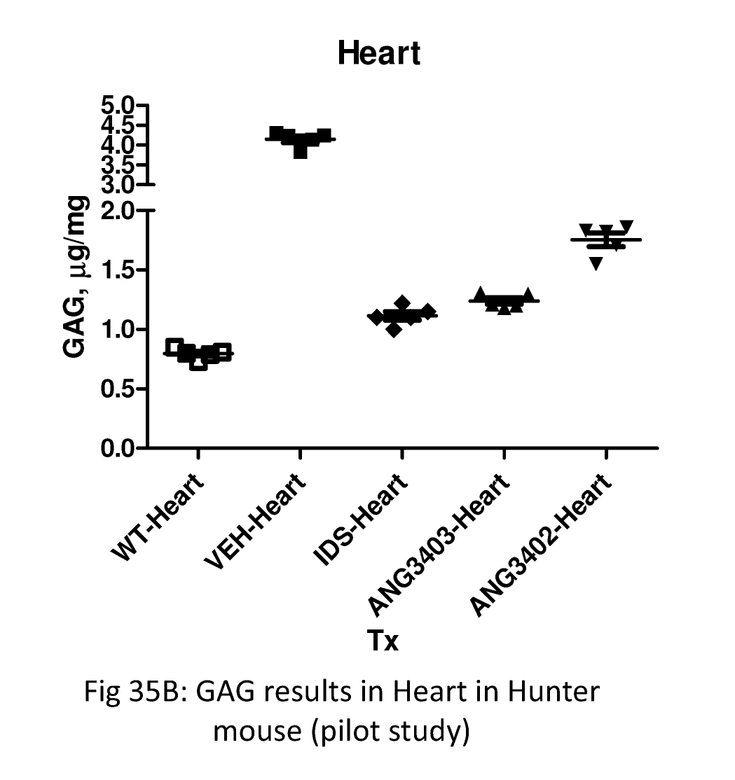

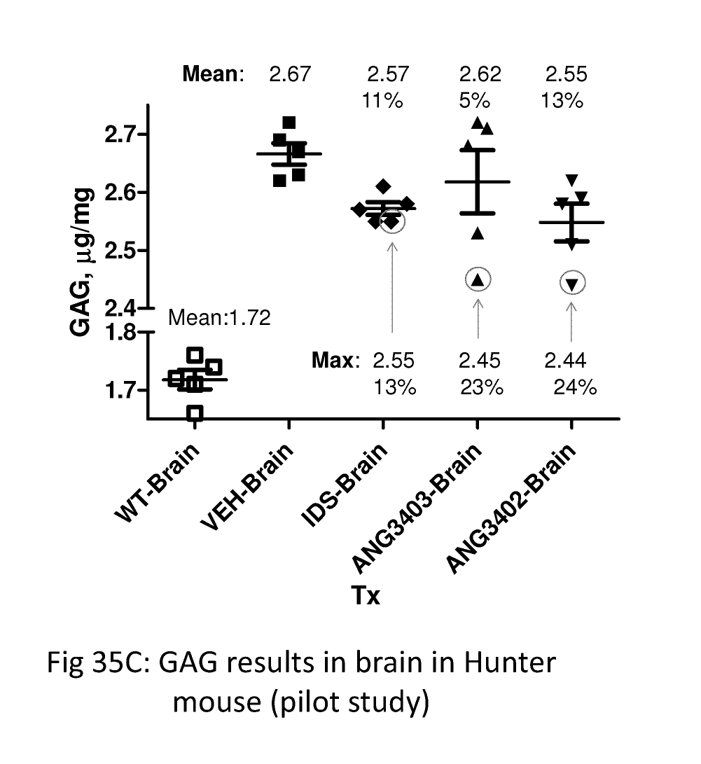

In liver (FIG. 35A) and heart (FIG. 35B), GAG concentration was significantly lower in hemizygous knock out mice dosed with JR-032 or conjugates compared to hemizygous knock out mice dosed with vehicle only. No significant difference in GAG concentration was observed in brain tissue in hemizygous mice dosed with vehicle or with JR-032 or conjugates (FIG. 35C).

### Example 19

### Effect of Conjugates on GAG Accumulation in the Brain of Hemizygous Iduronate-2-Sulfatase Knock Out Mice

### Protocol

Male hemizygous iduronate-2-sulfatase gene knock-out mice (supplied by Oriental BioService Inc.; Minamiyamashiro Laboratory) aged 18 weeks (on receipt) were dosed at a volume of 5 mL/kg body weight via injection into the caudal vein twice a week for 8 weeks according to Table 12. Male wild type animals (18 weeks old) were used in test group 1 as a control.

TABLE 12
GAG accumulation dosing schedule

| | Type of | Dosage | Dosing | No of |
|---|---|---|---|---|
| Test group | animal | (mg/kg) | frequency | animals |
| 1. Wild type | WT | Not dosed | Not dosed | 5 |
| 2. Vehicle | Hemi | 0 | Twice/week | 5 |
| 3. JR-032 | Hemi | 1 | twice/week | 5 |
| 4. JR-032 | Hemi | 2 | twice/week | 5 |
| 5. JR-032 | Hemi | 5 | twice/week | 5 |
| 6. 70-66-1B | Hemi | 1 | twice/week | 5 |
| 7. 70-66-1B | Hemi | 2 | twice/week | 5 |
| 8. 70-66-1B | Hemi | 5 | twice/week | 5 |
| 9. 68-32-2 | Hemi | 1 | twice/week | 5 |
| 10. 68-32-2 | Hemi | 2 | twice/week | 5 |
| 11. 68-32-2 | Hemi | 5 | twice/week | 5 |

JR-032, 68-32-2 or 70-66-1B were administered in vehicle (20 mM Sodium Phosphate, 137 mM NaCl, pH 6). In test group 2, vehicle only was administered.

1 week after completion of the 8 week administration, the auricle of the right atrium was cut open under 20% isoflurane anesthesia and about 30 mL saline was perfused from the left ventricle with a syring and a needle. After perfusion, the brain (cerebrum and cerebellum) was removed. The brain was divided into the right brain and left brain. The right brain was weighed and frozen and the left brain was immersed in 10% neutral buffered formalin.

The fixed left brains were trimmed sagitally and embedded in paraffin. The paraffin embedded tissue specimens were sectioned using a microtome to get 5 sections on the approx. 0.96+1-0.24 mm lateral site (thickness of sections: 4 μm). Twos ections were used for staining with H&E and LAMP-1.

The frozen tissue was freeze-dried (FZ-Compact, Asahi Life Science Co, Ltd.), cut into small pieces, and weighed. 0.5 mol/L tris HCl buffer solution (pH 7.5) containing 50 mg/mL actinase E was added such that the total additive amount of the solution is 1 ml per 100 mg dry weight of the tissues. The mixture was heated at 100° C. for 10 minutes using a dry bath incubator. Additional 0.5 mol/L tris HCl buffer solution (pH 7.5) containing 50 mg/mL actinase E was then added such that the ratio of the dry weight of the tissues to actinase E is 50 mg to 1 mg and the mixture was incubated at 60° C. for about 16 hours using a dry heat sterilizer. The mixture was then heated at 100° C. for 10 minutes using a dry bath incubator followed by centrifugation at 24° C. at about 20400 g for 10 minutes. The supernatant was removed and frozen for more than 12 hours. The supernatant was then thawed at room temperature and centrifuged again at 24° C. at about 20400 g for 10 minutes. The supernatant was removed and frozen.

A Wieslab® sGAG quantitative kit (EURO-DIAGNOSTICA) was used to determine GAG concentrations twice in 50 μl samples from brain, in a 50 μl blank sample (water for injection) and in 50 μl calibration samples (solutions of chondroitin sulfate B sodium salt in water for injection at concentrations of 640 μg/ml, 320 μg/ml, 160 μg/ml, 80 μg/ml 40 μg/ml and 20 μg/ml).

Briefly, 50 μl of 8 M guanidine-HCl was added to each sample and the mixture was allowed to react at room temperature for 15 minutes. 50 μl of SAT solution (0.3% H₂SO₄ and 0.75% Triton X-100) was then added and the mixture was allowed to react at room temperature for 15 minutes. 750 μl of Alcian Blue working solution (prepared by mixing water for injection, the SAT solution and Alcian Blue stock solution (0.1% H₂SO₄ and 0.4 M guandine HCl) at a ratio of 9:5:1) was added to each sample and the mixture was allowed to react at room temperature for 15 minutes. The samples were then centrifuged at 12600 g for 15 minutes at 24° C. and the supernatant was discarded. 500 μl of DMSO solution (40% dimethylsulphoxide and 0.05 M MgCl₂) was added to the tube and the contents of the tube were stirred with a mixer at room temperature for 15 minutes followed by centrifugation at 12600 g for 15 minutes at 24° C. The supernatant was discarded and 500 μl Gu-Prop (4 M guanidine-HCl, 33% 1-propanol and 0.25% Triton X-100) was added to each sample. The tubes were stirred with a mixer at room temperature for 15 minutes so that precipitates were completely dissolved. 200 μl of each sample was dispensed into wells in a 96 well microplate. A microplate reader was used to obtain absorbance values at a wavelength of 600 nm. GAG concentrations were calculated by a linear method with analysis software (KC4 v3.4 DS Pharma Biomedical Co., Ltd.). Mean values (μg/ml) of 2 measurements of GAG concentrations were calculated.

The accuracy of GAG concentrations of the calibration samples were calculated. When the accuracy and correlation coefficient were not within the evaluation criteria, GAG concentrations in the tissue samples were not calculated. (Evaluation criteria for accuracy: coefficient of variation is within 15% (within 20% for the 20 μg/ml sample). Evaluation criteria for correlation coefficient: 0.997 or higher (rounded to 4 decimal places)).

The GAG concentrations measured in the brain samples were converted to the concentration in the dry weight of brain by the following formula:

$$[ GAG ] d = \frac{[ GAG ] s \times A}{W}$$

[GAG]d=GAG concentration in dry tissue (μg/mg)
[GAG]s=GAG concentration in sample (μg/ml)
A=Additive amount (ml)
W=Dry tissue weight (mg)

### Results

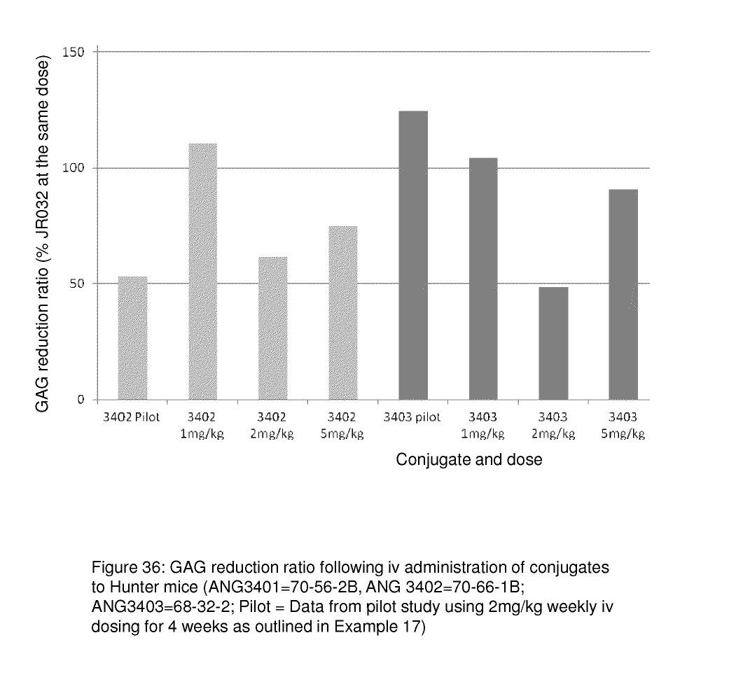

FIG. 36 is a graph showing GAG reduction at each dose for each conjugate (expressed as a percentage of the reduction achieved for JR-032). The GAG reduction achieved by the same conjugates in the study described in Example 17 are included in this graph for comparision).

### Other Embodiments

All patents, patent applications, and publications mentioned in this specification are herein incorporated by reference, including U.S. Provisional Application No. 61/732,145, filed Nov. 30, 2012, and U.S. Provisional Application No. 61/831,919 filed Jun. 6, 2013, to the same extent as if each independent patent, patent application, or publication was specifically and individually indicated to be incorporated by reference.

What is claimed is:

1. A compound comprising (a) a peptide or peptidic targeting moiety less than 150 amino acids and (b) an enzyme selected from the group consisting of iduronate-2-sulfatase (IDS), an IDS fragment having IDS activity, or an IDS analog, wherein said targeting moiety is capable of transporting said enzyme, fragment or analog across the blood brain barrier, wherein said compound exhibits IDS enzymatic activity, wherein said targeting moiety and said enzyme, fragment, or analog are joined by a linker, and wherein said linker joining said enzyme and said peptide targeting moiety can be formed by a click chemistry reaction between a click-chemistry reaction pair and the linker does not have the structure:

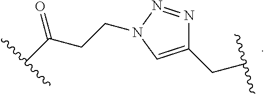 [SMILES: *Cc1cn(CCC(*)=O)nn1]

2. A compound of claim 1, wherein said linker is selected from the group consisting of a monofluorocyclooctyne (MFCO) containing linker, a difluorocyclooctyne (DECO) containing linker, a (DBCO) containing linker, a cyclooctyne (OCT) containing linker, a dibenzocyclooctyne (DIBO) containing linker, a biarylazacyclooctyne (BARAC) containing linker, a difluorobenzocyclooctyne (DIFBO) containing linker, and a bicyclo[6.1.0]nonyne (BCN) containing linker.

3. The compound of claim 1, wherein said targeting moiety comprises an amino acid sequence that is at least 70% identical to any of SEQ ID NOS:1-105 and 107-117.

4. The compound of claim 3, wherein said targeting moiety comprises the sequence of Angiopep-2 (SEQ ID NO:97).

5. The compound of claim 4, wherein said targeting moiety optionally comprises one or more D-isomers of an amino acid recited in SEQ ID NO: 97.

6. The compound of claim 1, wherein said targeting moiety comprises the formula Lys-Arg-X3-X4-X5-Lys (formula Ia), wherein:
X3 is Asn or Gln;
X4 is Asn or Gln; and
X5 is Phe, Tyr, or Trp;
wherein said targeting moiety optionally comprises one or more D-isomers of an amino acid recited in formula Ia.

7. The compound claim 1 or 2, wherein said targeting moiety comprises the formula Z1-Lys-Arg-X3-X4-X5-Lys-Z2 (formula Ib), wherein:
X3 is Asn or Gin;
X4 is Asn or Gin;
X5 is Phe, Tyr, or Trp;
Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and
Z2 is absent, Cys, Tyr, Tyr-Cys, Cys-Tyr, Thr-Glu-Glu-Tyr, or Thr-Glu-Glu-Tyr-Cys; and
wherein said targeting moiety optionally comprises one or more D-isomers of an amino acid recited in formula Ib, Z1, or Z2.

8. The compound of claim 7, wherein said targeting moiety comprises at least three D-isomers of an amino acid recited in formula Ib, Z1, or Z2.

9. The compound of claim 8, wherein said targeting moiety has the formula Thr-Phe-Phe-Tyr-Gly-Gly-Ser-D-Arg-Gly-D-Lys-D-Arg-Asn-Asn-Phe-Lys-Thr-Glu-Glu-Tyr.

10. The compound of claim 8, wherein said targeting moiety has the formula Thr-Phe-Phe-Tyr-Gly-Gly-Ser-D-Arg-Gly-D-Lys-D-Arg-Asn-Asn-Phe-D-Lys-Thr-Glu-Glu-Tyr.

11. The compound of claim 1, wherein said targeting moiety comprises the formula X1-X2-Asn-Asn-X5-X6 (formula IIa),
wherein:
X1 is Lys or D-Lys;
X2 is Arg or D-Arg;
X5 is Phe or D-Phe; and
X6 is Lys or D-Lys; and
wherein at least one of X1, X2, X5, or X6 is a D-amino acid.

12. The compound of claim 1, wherein said targeting moiety comprises the formula X1-X2-Asn-Asn-X5-X6-X7 (formula IIb),
wherein:
X1 is Lys or D-Lys;
X2 is Arg or D-Arg;
X5 is Phe or D-Phe;
X6 is Lys or D-Lys; and
X7 is Tyr or D-Tyr; and
wherein at least one of X1, X2, X5, X6, or X7 is a D-amino acid.

13. The compound of claim 1, wherein said targeting moiety comprises the formula Z1-X1-X2-Asn-Asn-X5-X6-X7-Z2 (formula IIc),
wherein:
X1 is Lys or D-Lys;
X2 is Arg or D-Arg;
X5 is Phe or D-Phe;
X6 is Lys or D-Lys;
X7 is Tyr or D-Tyr;
Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and
Z2 is absent, Cys, Tyr, Tyr-Cys, Cys-Tyr, Thr-Glu-Glu-Tyr, or Thr-Glu-Glu-Tyr-Cys;
wherein at least one of X1, X2, X5, X6, or X7 is a D-amino acid; and
wherein said targeting moiety optionally comprises one or more D-isomers of an amino acid recited in Z1 or Z2.

14. The compound of claim 1, wherein the linker is a bicyclo[6.1.0]nonyne (BCN) containing linker.

15. The compound of claim 1, wherein the linker is an monofluorocyclooctyne (MFCO) containing linker.

16. A compound having the general structure 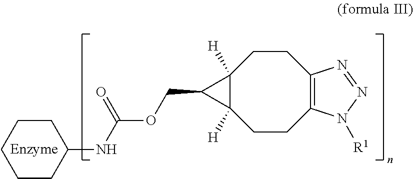
wherein R¹ is: 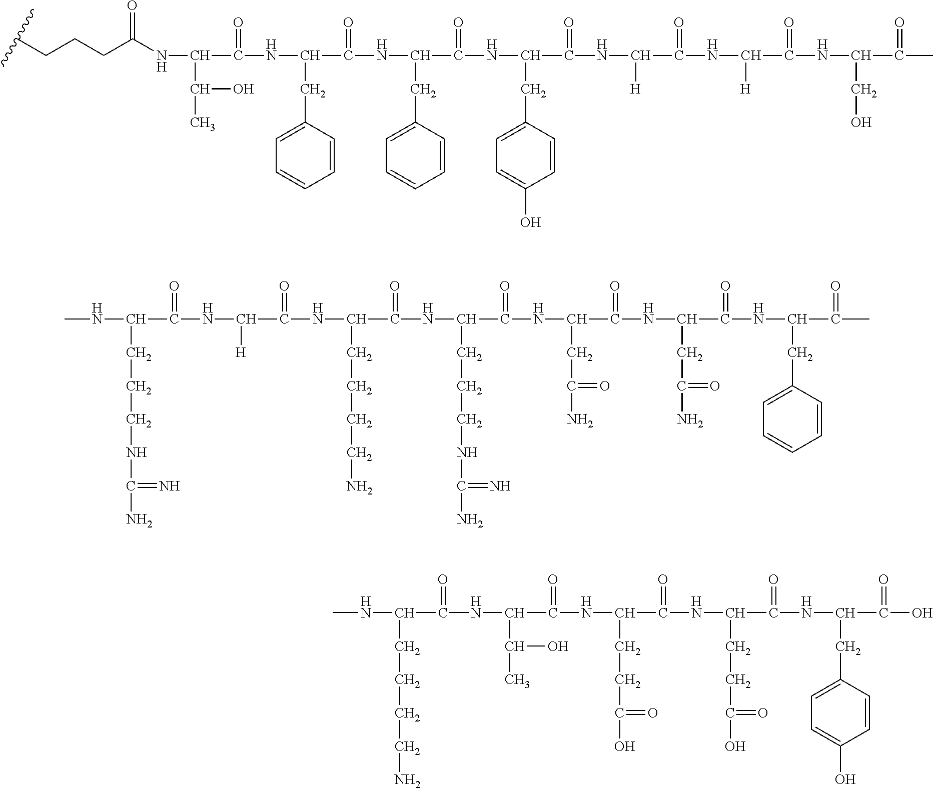
wherein enzyme represents IDS, an active fragment of IDS, or an active analog of IDS, where n is an integer between 1 and 6 and wherein the NH group attached to the enzyme is derived from the reaction of a primary amino group in the enzyme.

17. The compound of claim 16, wherein one or more NH groups attached to enzyme are derived from the primary amino groups of one or more lysine residues.

18. The compound of claim 17, wherein one or more NH groups attached to enzyme are derived from the primary amino groups of lysine 199 and/or lysine 376 corresponding to full length human IDS isoform a.

19. The compound of claim 16, wherein n is 1.

20. The compound of claim 16, wherein n is 2.

21. A population of compounds of formula III as defined in claim 16, wherein the average value of n is between 1 and 6.

22. A compound having the general structure 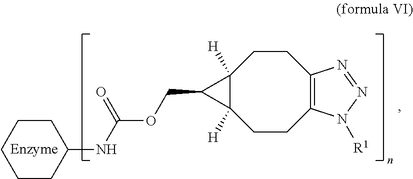
wherein R¹ is: 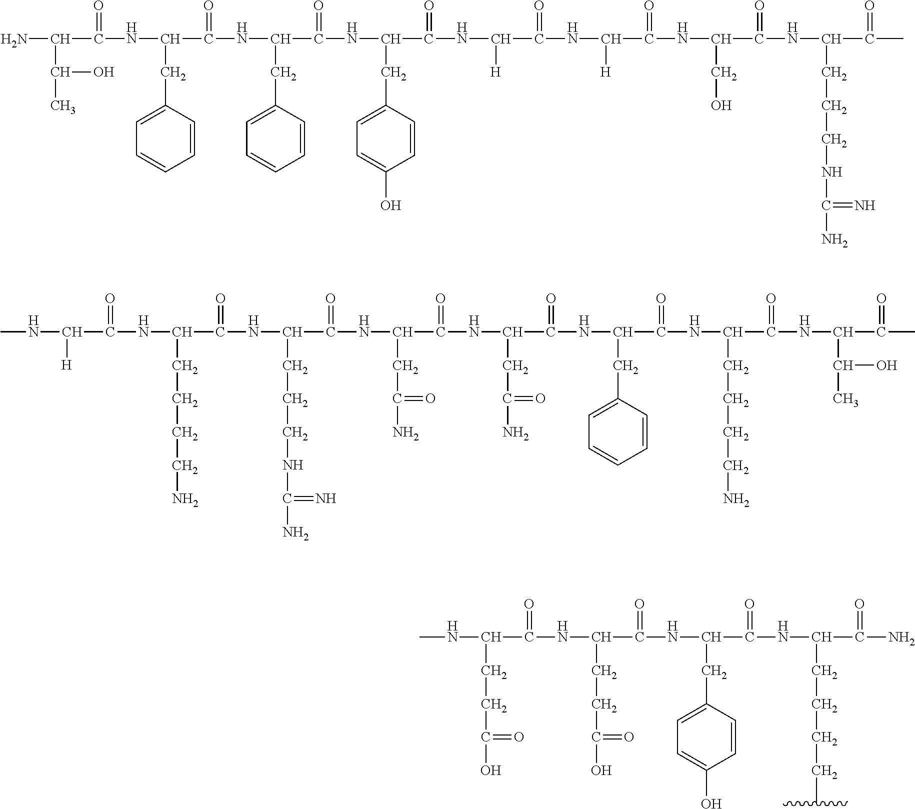
wherein enzyme represents IDS, an active fragment of IDS, or an active analog of IDS, where n is an integer between 1 and 6, and wherein the NH group attached to the enzyme is derived from the reaction of a primary amino group in the enzyme.

23. A population of compounds of formula VI as defined in claim 22, wherein the average value of n is between 1 and 6.

24. The population of compounds of claim 23, wherein the average value of n is about 2.3.

25. The compound or the population of compounds of claim 22, wherein one or more NH groups attached to enzyme are derived from the primary amino groups of one or more lysine residues.

26. The compound or population of compounds of claim 25 wherein one or more NH groups attached to enzyme are derived from the primary amino groups of lysine 199 and/or lysine 479 corresponding to full length human IDS isoform a.

27. A compound having the general structure 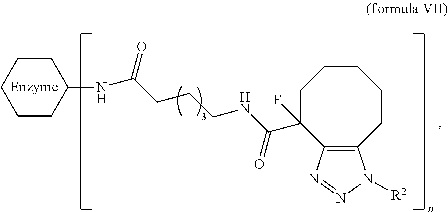
wherein R² is: 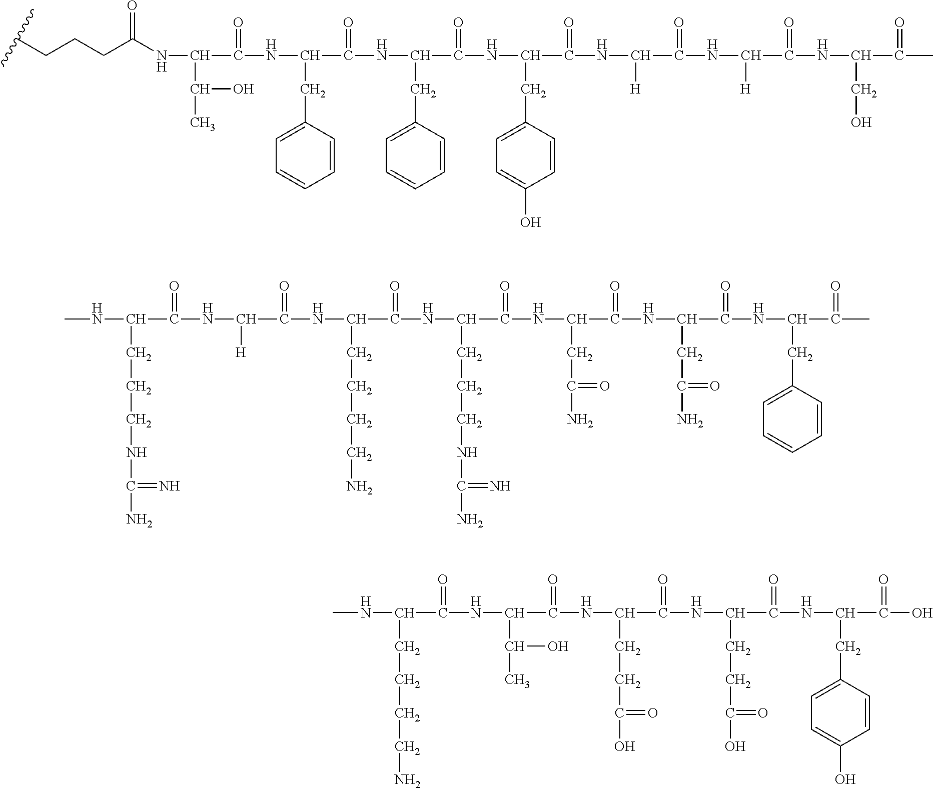
wherein enzyme represents IDS, an active fragment of IDS, or an active analog of IDS, where n is an integer between 1 and 6, and wherein the NH group attached to the enzyme is derived from the reaction of a primary amino group in the enzyme.

28. A population of compounds of formula VII as defined in claim 27, wherein the average value of n is between 1 and 6.

29. The population of compounds of claim 28, wherein the average value of n is about 2.3, about 4.4 or about 5.0.

30. A compound having the general structure 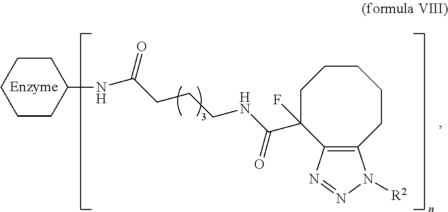
wherein R² is: 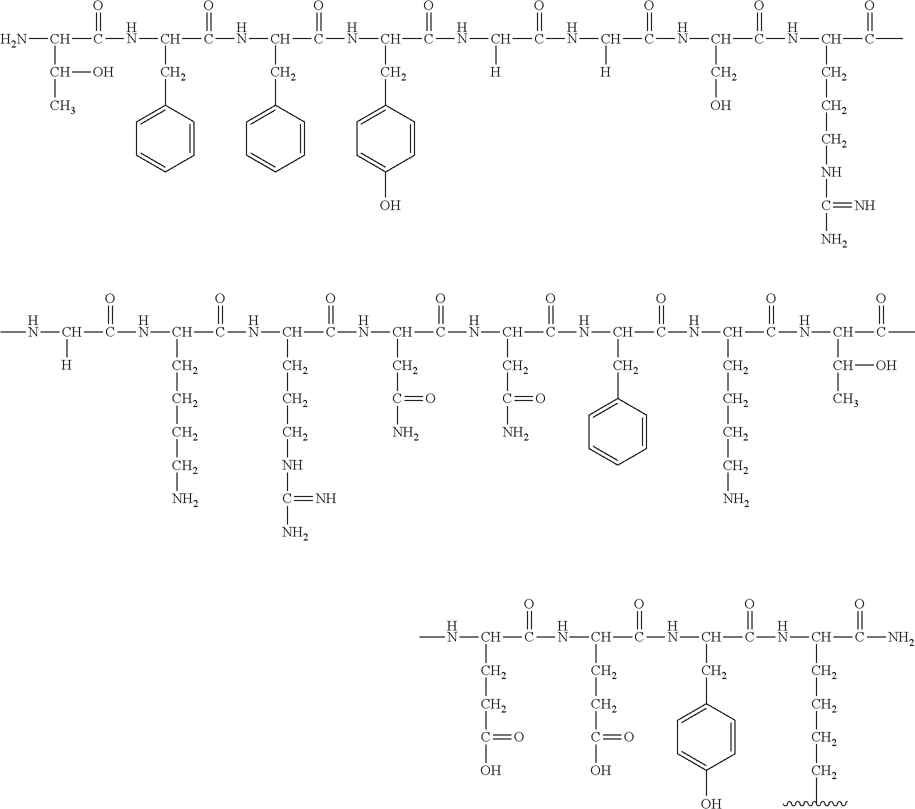
wherein enzyme represents IDS, an active fragment of IDS, or an active analog of IDS, wherein n is an integer between 1 and 6, and wherein the NH group attached to the enzyme is derived from the reaction of a primary amino group in the enzyme.

31. A population of compounds of formula VIII as defined in claim 30, wherein the average value of n is between 1 and 6.

32. The population of compounds of claim 31, wherein the average value of n is about 4.9.

33. The compound or the population of compounds of claim 30, wherein one or more NH groups attached to enzyme are derived from the primary amino groups of one or more lysine residues.

34. The compound or population of compounds of claim 33 wherein one or more NH groups attached to enzyme are derived from one or more of the primary amino groups of lysine 199, lysine 211 and lysine 376 corresponding to full length human IDS isoform a.

35. A compound having the general structure 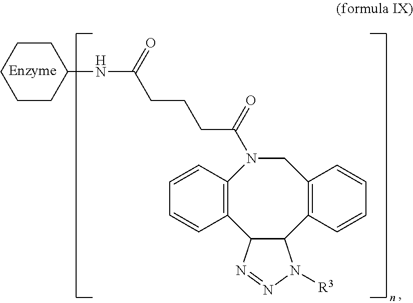
wherein R³ is: 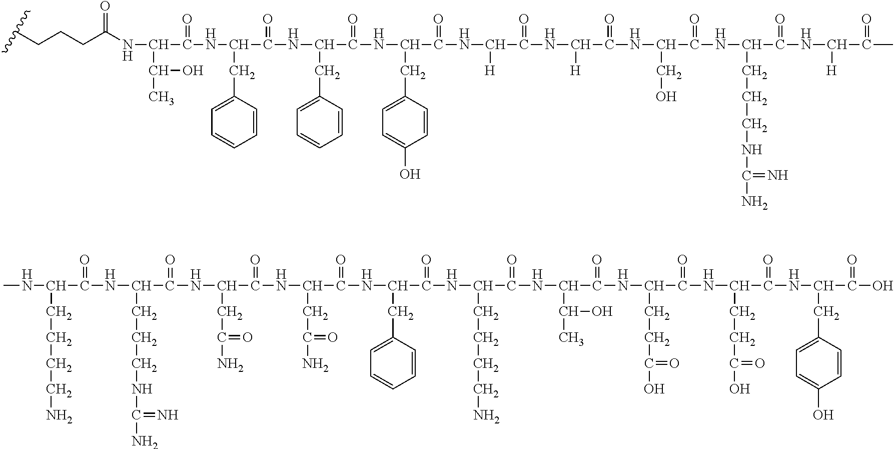
wherein enzyme represents IDS, an active fragment of IDS, or an active analog of IDS, wherein n is an integer between 1 and 6, and wherein the NH group attached to the enzyme is derived from the reaction of a primary amino group in the enzyme.

36. A population of compounds of formula IX as defined in claim 35, wherein the average value of n is between 1 and 6.

37. The population of compounds of claim 36, where the average value of n is about 1.3.

38. A compound having the general structure 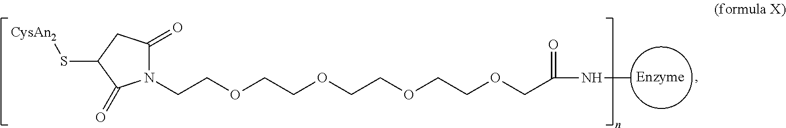
wherein enzyme represents IDS, an active fragment of IDS, or an active analog of IDS, wherein n is the number of Angiopep-2 moieties attached to IDS via the linker and is an integer between 1 and 6, the S moiety attached to An₂Cys represents the side chain sulfide on the cysteine in Angiopep-2-Cys, and wherein the NH group attached to the enzyme is derived from the reaction of a primary amino group in the enzyme.

39. A population of compounds of formula X as defined in claim 38, wherein the average value of n is between 0.5 and 6.

40. The population of compounds of claim 39, where the average value of n is about 0.8.

41. A compound having the general structure 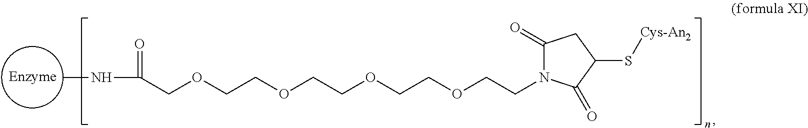
wherein enzyme represents IDS, an active fragment of IDS, or an active analog of IDS, wherein n is the number of Angiopep-2 moieties attached to IDS via the linker and is an integer between 1 and 6, wherein Cys-An₂ is Cys-Angiopep-2, the S moiety attached to Cys-An₂ represents the side chain sulfide on the cysteine in Cys-Angiopep-2, and the wherein the NH group attached to the enzyme is derived from the reaction of a primary amino group in the enzyme.

42. A population of compounds of formula XI as defined in claim 41, wherein the average value of n is between 0.5 and 6.

43. The population of compounds of claim 42, where the average value of n is about 0.9.

44. A compound having the general structure 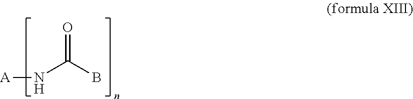
wherein A is an enzyme selected from the group consisting of iduronate-2-sulfatase (IDS), an IDS fragment having IDS activity, or an IDS analog having IDS activity; the NH group attached to A is derived from the reaction of a primary amino group in A; n is an integer between 1 and 8; and B is hydroxyl, optionally substituted C₁₋₁₀ alkyl, optionally substituted C₁₋₁₀alkenyl, optionally substituted alkynyl, optionally substituted aryl, heterocycle, optionally substituted C₁₋₁₀ alkoxy, optionally substituted C₁₋₁₀ alkylamino, optionally substituted C₃₋₁₀ cycloalkyl, optionally substituted C₄₋₁₀ cycloalkenyl, optionally substituted C₄₋₁₀ cycloalkynyl, an amino acid, or a peptide of 2 to 5 amino acids.

45. The compound of claim 44, wherein B is an amino acid, a peptide of 2 to 5 amino acids, or selected from:

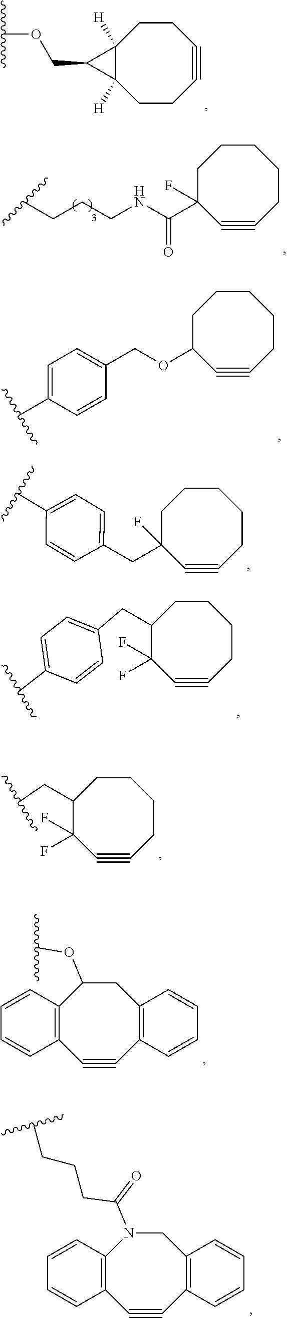

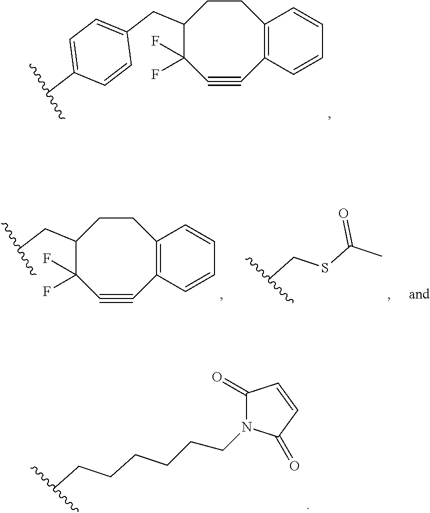

46. The compound of claim 44, wherein B is:

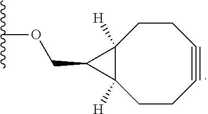 [SMILES: *OC[C@@H]1[C@@H]2CCC#CCC[C@@H]21]

47. A population of compounds of formula XIII as defined in claim 44, wherein the average value of n is between 1 and 8.

48. The compound or population of compounds of claim 46, wherein one or more NH groups attached to A are derived from the primary amino groups of one or more lysine residues.

49. The compound or population of compounds of claim 48, wherein one or more NH groups attached to A are derived from one or more of the primary amino groups of lysine 199, lysine 240, lysine 295, lysine 347, lysine 479 and lysine 483 corresponding to full length human IDS isoform a.

50. The compound of population of compounds of claim 49 wherein one or more NH groups attached to A are derived from one or more of the primary amino groups of lysine 199, lysine 479 and lysine 483 corresponding to full length human IDS isoform a.

51. The compound of claim 44, wherein B is:

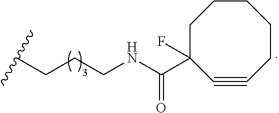

52. A population of compounds of formula XIII as defined in claim 51, wherein the average value of n is between 1 and 8.

53. The compound or population of compounds of claim 51, wherein one or more NH groups attached to A are derived from the primary amino groups of one or more lysine residues.

54. The compound or population of compounds of claim 53, wherein one NH group attached to A is derived from the primary amino groups of lysine 479 corresponding to full length human IDS isoform a.

55. The compound or population of compounds of claim 1, wherein IDS or said IDS fragment has the amino acid sequence of human IDS isoform a or a fragment thereof, or wherein said IDS analog has at least 70% identity to the sequence of full length human IDS isoform a.

56. The compound or population of compounds of claim 55, wherein IDS has the sequence of human IDS isoform a or the mature form of isoform a (amino acids 26-550 of isoform a).

57. A composition comprising one or more nanoparticles, wherein said nanoparticle is conjugated to the compound or population of compounds of claim 1.

58. A composition comprising a liposome formulation of the compound or population of compounds of claim 1.

59. A pharmaceutical composition comprising the compound or population of compounds of claim 1 and a pharmaceutically acceptable carrier.

60. A method of treating or treating prophylactically a subject having mucopolysaccharidosis Type II (MPS-II), said method comprising administering to said subject a compound or population of compounds of claim 1.

61. The method of claim 60, wherein said subject has the severe form of MPS-II.

62. The method of claim 60, wherein said subject has the attenuated form of MPS-II.

63. The method of claim 60, wherein said subject has neurological symptoms.

64. The method of claim 60, wherein said subject starts treatment under five years of age.

65. The method of claim 64, wherein said subject starts treatment under three years of age.

66. The method of claim 65, wherein said subject is an infant.

67. The method of claim 60, wherein said administering comprises parenteral administration.
